(19) United States
(12) Patent Application Publication
(10) Pub. No.: US 2022/0347415 A1
(43) Pub. Date: Nov. 3, 2022

(54) PATIENT INTERFACE AND COMPONENT DETECTION, MONITORING AND REPLACEMENT

(71) Applicant: ResMed Pty Ltd, Bella Vista NSW (AU)

(72) Inventors: Peter Farlow, Sydney (AU); Faizan Javed, Sydney (AU); Ryan Michael Kirkpatrick, Sydney (AU); Gregory Robert Peake, Sydney (AU); Kenneth John Taylor, Sydney (AU); Caitlin Isa Davie, Sydney (AU); Amanda Marion Chancellor, Sydney (AU); Jamal Moussa, Sydney (AU); Michelle Su, Sydney (AU)

(21) Appl. No.: 17/733,374

(22) Filed: Apr. 29, 2022

(30) Foreign Application Priority Data
Apr. 30, 2021 (AU) 2021901278

(51) Int. Cl. A61M 16/06; A61M 16/00

(52) U.S. Cl. CPC A61M 16/06; A61M 16/0003; A61M 2016/0027; A61M 2016/0033

(57) ABSTRACT

A method for determining that a patient interface component comprising a vent has been replaced between therapy sessions of treatment of sleep disordered breathing, the method comprising: acquiring or receiving first vent flow rate data representing one or more estimated first vent flow rates of gas through a first vent of a patient interface in use during a first therapy session; acquiring or receiving second vent flow rate data representing one or more estimated second vent flow rates of gas through a second vent of a patient interface in use during a second therapy session after the first therapy session; and identifying, by comparison of the second vent flow rate data to the first vent flow rate data, a difference in resistance to flow through the first vent than through the second vent indicating that the second vent is not the same vent as the first vent.

### 1 CROSS-REFERENCE TO RELATED APPLICATIONS

This application claims priority to Australian Patent Application No. 2021901278, filed 30 Apr. 2021, the entire contents of which are hereby incorporated by reference herein.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

The present technology relates generally to systems and methods for monitoring components of a respiratory pressure therapy system; more particularly the present technology relates to methods and systems for determining that components of a respiratory pressure therapy system may require replacement.

### 2.2 Description of the Related Art

### 2.2.1 Respiratory Disorders and Therapy

A range of respiratory disorders exist. Certain disorders may be characterised by particular events, e.g. apneas, hypopneas, and hyperpneas. Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders. Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass). CPAP, NIV and IV are examples of respiratory pressure therapy.

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat OSA, CSR, respiratory failure, COPD, and other respiratory disorders. As an alternative to constant flow rate, the treatment flow rate may follow a profile that varies over the respiratory cycle.

Another form of flow therapy is long-term oxygen therapy (LTOT) or supplemental oxygen therapy. For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

Another form of respiratory therapy is oxygen concentration. An oxygen concentrator is a device that concentrates the amount of oxygen in a gas supply to provide an oxygen-enriched flow of breathable gas to a patient. Some forms of oxygen concentrators operate by taking ambient air and selectively reducing its nitrogen content to produce the oxygen-enriched flow of breathable gas.

Another form of respiratory therapy is ventilation. A ventilator is a device that causes breathable air to move into and/or out of the lungs to enable a patient to breathe where the patient is unable to breathe themselves, or requires assistance to do so. A ventilator creates the flow of air through a mechanical mechanism.

### 2.2.2 Respiratory Therapy Systems

The above respiratory therapies may be provided by a respiratory therapy system or device. Such systems and devices may also be used to screen, diagnose, or monitor a condition without treating it. A respiratory therapy system may comprise a Respiratory Pressure Therapy Device (RPT device), an air circuit, a humidifier, a patient interface, an oxygen source, and/or data management.

### 2.2.2.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Patient interfaces may include a seal-forming structure. Since it is in direct contact with the patient's face, the shape and configuration of the seal-forming structure can have a direct impact on the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fits is comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the patient's face, and additional force will be required to force the patient interface against the patient's face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak excessively. Furthermore, if the shape of the seal-forming structure does not match that of the patient, it may crease or buckle in use, giving rise to excessive leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 2.2.2.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. The flow of air may be pressurised. Examples of RPT devices include a CPAP device, NIV device, HFT device, oxygen concentrator, and a ventilator.

### 2.2.2.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.2.4 Humidifier

Delivery of a flow of air without humidification may cause drying of airways. The use of a humidifier with an RPT device and the patient interface produces humidified gas that minimizes drying of the nasal mucosa and increases patient airway comfort. In addition in cooler climates, warm air applied generally to the face area in and about the patient interface is more comfortable than cold air. Humidifiers therefore often have the capacity to heat the flow of air was well as humidifying it.

### 2.2.2.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

### 2.2.2.6 Vent

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focused airflow.

ResMed Limited has developed a number of improved mask vent technologies. See International Patent Application Publication No. WO 1998/034,665; International Patent Application Publication No. WO 2000/078,381; U.S. Pat. No. 6,581,594; US Patent Application Publication No. US 2009/0050156; US Patent Application Publication No. 2009/0044808.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

Another aspect of the present technology relates to systems for detecting patient interface or patient interface component replacement, determining when patient interface or patient interface component replacement is required, estimating age of a patient interface, determining that a patient is using an HMX and/or related methods.

### 3.1 Gas Washout Vent Flow

3.1.1 Determining that a Vent Component has been Replaced Based on Vent Flow

One aspect of the present technology comprises a method for determining that a patient interface component comprising a vent has been replaced between therapy sessions of treatment of sleep disordered breathing, the method comprising:

acquiring or receiving first vent flow rate data, the first vent flow rate data representing one or more estimated first vent flow rates of gas through a first vent of a patient interface in use during a first therapy session;

acquiring or receiving second vent flow rate data, the second vent flow rate data representing one or more estimated second vent flow rates of gas through a second vent of a patient interface in use during a second therapy session after the first therapy session;

identifying, by comparison of the second vent flow rate data to the first vent flow rate data, a difference in resistance to flow through the first vent than through the second vent indicating that the second vent is not the same vent as the first vent.

In examples:
the difference in resistance to flow is a greater resistance to flow through the second vent than through the first vent;
the first vent flow rate data represents a plurality of estimated first vent flow rates each corresponding to a respective one of a plurality of therapy pressures, and the second vent flow rate data represents a plurality of estimated second vent flow rates each corresponding to a respective one of the plurality of therapy pressures;
the method comprises identifying the difference in resistance to flow by determining that for each one of the plurality of therapy pressures, the corresponding second vent flow rate is different to the corresponding first vent flow rate;
each one of the plurality of therapy pressures is within the range of 3-30 cmH₂O; each one of the therapy pressures is within the range of 5-20 cmH₂O;
the first vent flow rate data represents an estimated first vent flow rate corresponding to a predetermined therapy pressure, and the second vent flow rate data represents an estimated second vent flow rate corresponding to the predetermined therapy pressure;
the method comprises determining that the second vent flow rate is different to the first vent flow rate;
the predetermined therapy pressure is within the range of 3-30 cmH₂O; the predetermined therapy pressure is within the range of 5-20 cmH₂O;
the step of acquiring or receiving the first vent flow rate data comprises acquiring the first vent flow rate data and the step of acquiring or receiving the second vent flow rate data comprises acquiring the second vent flow rate data, wherein the steps of acquiring the first vent flow rate data and acquiring the second vent flow rate data are performed by a respiratory pressure therapy device providing a pressurised flow of breathable gas to the patient interface in use during the first therapy session and to the patient interface in use during the second therapy session;
the step of identifying the difference in resistance is performed by the respiratory pressure therapy device;
the method comprises transmitting the first vent flow rate data and the second vent flow rate data to a server, and the step of identifying the difference in resistance is performed by a server; and/or
the step of acquiring or receiving the first vent flow rate data comprises receiving the first vent flow rate data and the step of acquiring or receiving the second vent flow rate data comprises receiving the second vent flow rate data, wherein the steps of receiving the first vent flow rate data and receiving the second vent flow rate data are performed by a server, and the step of identifying the difference in resistance is performed by the server.

Another aspect of the present technology comprises a method for monitoring for replacement of a patient interface component comprising a vent between therapy sessions of treatment of sleep disordered breathing, the method comprising:

acquiring or receiving first vent flow rate data during a first therapy session, the first vent flow rate data representing one or more estimated first vent flow rates of gas through a vent of a patient interface in use during the first therapy session;

acquiring or receiving second vent flow rate data during a second therapy session after the first therapy session, the second vent flow rate data representing one or more estimated second vent flow rates of gas through a vent of a patient interface in use during the second therapy session;

checking for, by comparison of the second vent flow rate data to the first vent flow rate data, a difference in resistance to flow through the vent of the patient interface in use during the second therapy session than through the vent of the patient interface in use during the first therapy session.

In examples:
the difference in resistance to flow is a greater resistance to flow through the vent of the patient interface in use during the second therapy session than through the vent of the patient interface in use during the first therapy session;
the first vent flow rate data represents a plurality of estimated first vent flow rates each corresponding to a respective one of a plurality of therapy pressures, and the second vent flow rate data represents a plurality of estimated second vent flow rates each corresponding to a respective one of the plurality of therapy pressures;
the step of checking for the difference in resistance to flow comprises checking for a difference, for each one of the plurality of therapy pressures, between the corresponding second vent flow rate and the corresponding first vent flow rate;
each one of the plurality of therapy pressures is within the range of 3-30 cmH₂O; each one of the therapy pressures is within the range of 5-20cmH₂O;
the first vent flow rate data represents an estimated first vent flow rate corresponding to a predetermined therapy pressure, and the second vent flow rate data represents an estimated second vent flow rate corresponding to the predetermined therapy pressure;
the step of checking for the difference in resistance to flow comprises checking for a difference between the second flow rate and the first flow rate;
the predetermined therapy pressure is within the range of 3-30 cmH₂O; the predetermined therapy pressure is within the range of 5-20cmH₂O;

In further examples:
the step of acquiring or receiving the first vent flow rate data comprises acquiring the first vent flow rate data and the step of acquiring or receiving the second vent flow rate data comprises acquiring the second vent flow rate data, wherein the steps of acquiring the first vent flow rate data and acquiring the second vent flow rate data are performed by a respiratory pressure therapy device providing a pressurised flow of breathable gas to the patient interface in use during the first therapy session and to the patient interface in use during the second therapy session;
the step of checking for the difference in resistance to flow is performed by the respiratory pressure therapy device;
the method comprises transmitting the first vent flow rate data and the second vent flow rate data to a server, and the step of checking for the difference in resistance to flow is performed by the server; and/or the step of acquiring or receiving the first vent flow rate data comprises receiving the first vent flow rate data and the step of acquiring or receiving the second vent flow rate data comprises receiving the second vent flow rate data, wherein the steps of receiving the first vent flow rate data and receiving the second vent flow rate data are performed by a server, and the step of checking for the difference in resistance to flow is performed by the server.
   3.1.2 Determining that a Vent Component Requires Replacement Based on Gas Washout Vent Flow

Another aspect of the present technology comprises a method of determining that a patient interface component comprising a vent requires replacement, the method comprising:

acquiring or receiving therapy vent flow rate data during a therapy session, the therapy vent flow rate data representing one or more estimated vent flow rates of gas through a vent of a therapy patient interface in use during the therapy session;

comparing the therapy vent flow rate data with reference vent flow rate data, the reference vent flow rate data representing one or more reference vent flow rates of gas through a reference vent;

determining, based on the comparison of the therapy vent flow rate data to the reference vent flow rate data, that replacement of a patient interface component comprising the vent of the therapy patient interface is required.

In examples:
the therapy vent flow rate data represents a plurality of estimated therapy vent flow rates each corresponding to a respective one of a plurality of therapy pressures, and the reference vent flow rate data represents a plurality of reference vent flow rates each corresponding to a respective one of the plurality of therapy pressures;
each one of the plurality of therapy pressures is within the range of 3-30 cmH₂O; each one of the therapy pressures is within the range of 5-20cmH₂O;
the reference vent has the behaviour of a vent in an unused patient interface and the step of determining that replacement is required comprises determining that for each one of the plurality of therapy pressures, the corresponding therapy vent flow rate is greater than the corresponding reference vent flow rate by a replacement threshold amount;
the reference vent has the behaviour of a vent in a used patient interface and the step of determining that replacement is required comprises determining that for each one of the plurality of therapy pressures, the corresponding therapy vent flow rate is substantially equal to or greater than the corresponding reference vent flow rate;

In further examples:
the therapy vent flow rate data represents an estimated therapy vent flow rate corresponding to a predetermined therapy pressure, and the reference vent flow rate data represents a reference vent flow rate corresponding to the predetermined therapy pressure;
the predetermined therapy pressure is within the range of 3-30 cmH₂O; the predetermined therapy pressure is within the range of 5-20cmH₂O;
the reference vent has the behaviour of a vent in an unused patient interface and the step of determining that replacement is required comprises determining that the therapy vent flow rate is greater than the reference vent flow rate by a replacement threshold amount;
the reference vent has the behaviour of a vent in a used patient interface requiring replacement and the step of determining that replacement is required comprises determining that the therapy vent flow rate is substantially equal to or greater than the reference vent flow rate;

In further examples:
the step of acquiring or receiving the therapy vent flow rate data comprises acquiring the therapy vent flow rate data and is performed by a respiratory pressure therapy device providing a pressurised flow of breathable gas to the therapy patient interface during the therapy session;
the step of comparing the therapy vent flow rate data with reference vent flow rate data and the step of determining that replacement is required are performed by the respiratory pressure therapy device;
the method comprises transmitting the therapy vent flow rate data to a server and the step of comparing the therapy vent flow rate data with reference vent flow rate data and the step of determining that replacement is required are performed by the server; and/or the step of acquiring or receiving the therapy vent flow rate data comprises receiving the therapy vent flow rate data and is performed by a server, and the step of comparing the therapy vent flow rate data with reference vent flow rate data and the step of determining that replacement is required are performed by the server.

Another aspect of the present technology comprises a method for checking whether a patient interface component comprising a vent requires replacement, the method comprising:

acquiring or receiving therapy vent flow rate data during a therapy session, the therapy vent flow rate data representing one or more estimated vent flow rates of gas through a vent of a therapy patient interface in use during the therapy session;

comparing the therapy vent flow rate data with reference vent flow rate data, the reference vent flow rate data representing one or more reference flow rates of gas through a reference vent;

determining, based on the comparison of the therapy vent flow rate data to the reference vent flow rate data, whether or not replacement of a patient interface component comprising the vent of the therapy patient interface is required.

In examples:
the therapy vent flow rate data represents a plurality of estimated therapy vent flow rates each corresponding to a respective one of a plurality of therapy pressures, and the reference vent flow rate data represents a plurality of reference vent flow rates each corresponding to a respective one of the plurality of therapy pressures;
each one of the plurality of therapy pressures is within the range of 3-30 cmH₂O; each one of the therapy pressures is within the range of 5-20 cmH₂O;
the reference vent has the behaviour of a vent in an unused patient interface and the step of determining whether or not replacement is required comprises determining whether, for each one of the plurality of therapy pressures, the corresponding therapy vent flow rate is greater than the corresponding reference vent flow rate by a replacement threshold amount;
the reference vent has the behaviour of a vent in a used patient interface requiring replacement and the step of determining whether or not replacement is required comprises determining whether, for each one of the plurality of therapy pressures, the corresponding therapy vent flow rate is substantially equal to or greater than the corresponding reference vent flow rate;

In further examples:
the therapy vent flow rate data represents an estimated therapy vent flow rate corresponding to a predetermined therapy pressure, and the reference vent flow rate data represents a reference vent flow rate corresponding to the predetermined therapy pressure;
the predetermined therapy pressure is within the range of 3-30 cmH₂O; the predetermined therapy pressure is within the range of 5-20cmH₂O;
the reference vent has the behaviour of a vent in an unused patient interface and the step of determining whether or not replacement is required comprises determining whether the therapy vent flow rate is greater than the reference vent flow rate by a replacement threshold amount;
the reference vent has the behaviour of a vent in a used patient interface requiring replacement and the step of determining whether or not replacement is required comprises determining whether the therapy vent flow rate is substantially equal to or greater than the reference vent flow rate;

In further examples:
the step of acquiring or receiving the therapy vent flow rate data comprises acquiring the therapy vent flow rate data and is performed by a respiratory pressure therapy device providing a pressurised flow of breathable gas to the therapy patient interface during the therapy session;
the step of comparing the therapy vent flow rate data with reference vent flow rate data and the step of determining whether or not replacement is required are performed by the respiratory pressure therapy device;
the method comprises transmitting the therapy vent flow rate data to a server and the step of comparing the therapy vent flow rate data with reference vent flow rate data and the step of determining whether or not replacement is required are performed by the server; and/or the step of acquiring or receiving the therapy vent flow rate data comprises receiving the therapy vent flow rate data and is performed by a server, and the step of comparing the therapy vent flow rate data with reference vent flow rate data and the step of determining whether or not replacement is required are performed by the server.

### 3.1.3 Estimating Age of a Patient Interface Based on Gas Washout Vent Flow

Another aspect of the present technology comprises a method for estimating age of a patient interface component comprising a vent, the method comprising:

acquiring or receiving therapy vent flow rate data during a therapy session, the therapy vent flow rate data representing one or more estimated vent flow rates of gas through a vent of a therapy patient interface in use during the therapy session;

comparing the therapy vent flow rate data with reference vent flow rate data, the reference vent flow rate data representing one or more reference vent flow rates of gas through a reference vent;

determining a magnitude of difference in resistance to flow through the vent of the therapy patient interface than through the reference vent;

estimating an age of a patient interface component comprising the vent of the therapy patient interface based on the magnitude of difference in resistance to flow.

In examples:
the reference vent has the behaviour of a vent in an unused patient interface;
the method comprises determining that the magnitude of difference in resistance to flow is substantially zero and estimating that the age of the patient interface component comprising the vent is substantially zero, indicating that the patient interface component comprising the vent is unused;
the method comprises identifying, by comparison of the therapy vent flow rate data to the reference vent flow rate data, a lesser resistance to flow through the vent of the therapy patient interface than through the reference vent;
the step of estimating the age comprises calculating the age based on an expected rate of change over time of the magnitude of difference in resistance to flow through the vent of the therapy patient interface than through the reference vent;

In further examples:
the reference vent has the behaviour of a vent in a used patient interface having an age at which replacement is required;
the method comprises determining that the magnitude of difference in resistance to flow is substantially zero and estimating that the age of the patient interface component comprising the vent is equal to or greater than the age at which patient interface replacement is required;
the method comprises identifying, by comparison of the therapy vent flow rate data to the reference vent flow rate data, a greater resistance to flow through the vent of the therapy patient interface than through the reference vent;
the step of estimating the age of the patient interface component comprising the vent comprises calculating the age based on an expected rate of change over time of the magnitude of the difference in resistance to flow through the vent of the therapy patient interface than through the reference vent;

In further examples:
the therapy vent flow rate data represents a plurality of estimated flow rates corresponding to a respective one of a plurality of therapy pressures, and the reference vent flow rate data represents a plurality of reference flow rates each corresponding to a respective one of the plurality of therapy pressures;
the step of determining the magnitude of difference in resistance to flow comprises, for each one of the plurality of therapy pressures, calculating a difference between the therapy flow rate and the corresponding reference flow rate;
each one of the plurality of therapy pressures is within the range of 3-30 cmH₂O; each one of the therapy pressures is within the range of 5-20cmH₂O;
the therapy vent flow rate data represents an estimated first flow rate corresponding to a predetermined therapy pressure, and the reference vent flow rate data represents a reference flow rate corresponding to the predetermined therapy pressure;
the step of determining the magnitude of difference in resistance to flow comprises calculating a difference between the therapy flow rate and the reference flow rate;
the predetermined therapy pressure is within the range of 3-30 cmH₂O; the predetermined therapy pressure is within the range of 5-20cmH₂O;

In further examples:
the step of acquiring or receiving the therapy vent flow rate data comprises acquiring the therapy vent flow rate data and is performed by a respiratory pressure therapy device providing a pressurised flow of breathable gas to the therapy patient interface;
the step of comparing the therapy vent flow rate data with the reference vent flow rate data performed by the respiratory pressure therapy device;
the step of determining the magnitude of difference in resistance to flow is performed by the respiratory pressure therapy device;
the step of estimating the age based on the magnitude of difference in resistance to flow is performed by the respiratory pressure therapy device;
the method comprises transmitting the therapy vent flow rate data to a server;
the step of comparing the therapy vent flow rate data with the reference vent flow rate data is performed by the server;
the step of determining the magnitude of difference in resistance to flow is performed by the server;
the step of estimating the age based on the magnitude of difference in resistance to flow is performed by the server; and/or the step of acquiring or receiving the therapy vent flow rate data comprises receiving the therapy vent flow rate data and is performed by a server, and the server performs the steps of comparing the therapy vent flow rate data with the reference vent flow rate data, determining the magnitude of difference in resistance to flow and estimating the age based on the magnitude of difference in resistance to flow.

### 3.2 AAV Behaviour

3.2.1 Determining that an AAV Component has been Replaced Based on AAV Behaviour

Another aspect of the present technology comprises a method for determining that a patient interface component comprising an anti-asphyxia valve (AAV) has been replaced between therapy sessions of treatment of sleep disordered breathing, the method comprising:

acquiring or receiving first vent flow rate data during a first therapy session, the first vent flow rate data representing estimated flow rates of gas to atmosphere including through a first AAV of a patient interface in use during ramping up of interface pressure during the first therapy session;

acquiring or receiving second vent flow rate data during a second therapy session after the first therapy session, the second vent flow rate data representing estimated flow rates of gas to atmosphere including through a second AAV of a patient interface in use during ramping up of interface pressure during the second therapy session;

identifying, by comparison of the second vent flow rate data to the first vent flow rate data, a difference in behaviour between the first AAV and the second AAV during ramping up of interface pressure.

In examples:
the difference in behaviour comprises the second AAV closing during ramping up of interface pressure during the second therapy session one or more times more than the first AAV closes during ramping up of interface pressure during the first therapy session;
the difference in behaviour comprises the second AAV reopening and closing after closing a first time during ramping up of interface pressure during the second therapy session, while the first AAV closes only once during ramping up of interface pressure during the first therapy session;
the step of identifying the difference in behaviour comprises:
   identifying a first number of reductions in flow rate to atmosphere in response to increased interface pressure during ramping up of interface pressure during the first therapy session, each of the reductions indicating a closure of the first AAV; and
   identifying a second number of reductions in flow rate to atmosphere in response to increased interface pressure during ramping up of interface pressure during the second therapy session, each of the reductions indicating a closure of the second AAV, the second number of reductions being greater than the first number of reductions;
the step of identifying the difference in behaviour comprises:
   identifying only one reduction in flow rate to atmosphere in response to increasing interface pressure during ramping up of interface pressure during the first therapy session, the one reduction indicating a closure of the first AAV; and
   identifying two or more reductions in flow rate in response to increasing interface pressure during ramping up of interface pressure during the second therapy session, each reduction indicating a closure of the second AAV;

In further examples:
the step of acquiring or receiving the first vent flow rate data comprises acquiring the first vent flow rate data and the step of acquiring or receiving the second flow rate data comprises acquiring the second vent flow rate data, wherein the steps of acquiring the first vent flow rate data and acquiring the second vent flow rate data are performed by a respiratory pressure therapy device providing a pressurised flow of breathable gas to the patient interface in use during the first therapy session and to the patient interface in use during the second therapy session;
the step of identifying the difference in behaviour is performed by the respiratory pressure therapy device;
the method comprises transmitting the first vent flow rate data and second vent flow rate data to a server and the step of identifying the difference in behaviour is performed by the server;
the step of acquiring or receiving the first vent flow rate data comprises receiving the first vent flow rate data and the step of acquiring or receiving the second vent flow rate data comprises receiving the second flow rate data, wherein the steps of receiving the first vent flow rate data and receiving the second vent flow rate data are performed by a server, and the step of identifying the difference in behaviour is performed by the server.

Another aspect of the present technology comprises a method for monitoring for replacement of a patient interface component comprising an anti-asphyxia valve (AAV), the method comprising:

acquiring or receiving first vent flow rate data during a first therapy session, the first vent flow rate data representing estimated flow rates of gas to atmosphere including through an AAV of a patient interface in use during the first therapy session and during ramping up of interface pressure;

acquiring or receiving second vent flow rate data during a second therapy session after the first therapy session, the second vent flow rate data representing estimated flow rates of gas to atmosphere including through an AAV of a patient interface in use during the second therapy session and during ramping up of interface pressure;

checking for, by comparison of the second vent flow rate data to the first vent flow rate data, a difference in behaviour between the AAV of the patient interface in use during the first therapy session and the AAV of the patient interface in use during the second therapy session, during ramping up of interface pressure.

In examples:
the difference in behaviour comprises the AAV of the patient interface in use during the second therapy session closing during ramping up of interface pressure one or more times more than the AAV of the patient interface in use during the first therapy session closes during ramping up of interface pressure;
the difference in behaviour comprises the AAV of the patient interface in use during the second therapy session reopening and closing after closing a first time during ramping up of interface pressure during the second therapy session, while the AAV of the patient interface in use during the first therapy session closes only once during ramping up of interface pressure during the first therapy session;
the step of checking for the difference in behaviour comprises:
   identifying a first number of reductions in flow rate to atmosphere in response to increased interface pressure during ramping up of interface pressure during the first therapy session, each of the reductions indicating a closure of the AAV of the patient interface in use during the first therapy session; and
   identifying a second number of reductions in flow rate to atmosphere in response to increased interface pressure during ramping up of interface pressure during the second therapy session, each of the reductions indicating a closure of the AAV of the patient interface in use during the second therapy session, the second number of reductions being greater than the first number of reductions;
the step of checking for the difference in behaviour comprises:
   identifying only one reduction in flow rate to atmosphere in response to increasing interface pressure during ramping up of interface pressure during the first therapy session, the one reduction indicating a closure of the AAV of the patient interface in use during the first therapy session; and
   identifying two or more reductions in flow rate to atmosphere in response to increasing interface pressure during ramping up of interface pressure during the second therapy session, each reduction indicating a closure of the AAV of the patient interface in use during the second therapy session;
      In further examples:
the step of acquiring or receiving the first vent flow rate data comprises acquiring the first vent flow rate data and the step of acquiring or receiving the second flow rate data comprises acquiring the second vent flow rate data, wherein the steps of acquiring the first vent flow rate data and acquiring the second vent flow rate data are performed by a respiratory pressure therapy device providing a pressurised flow of breathable gas to the patient interface in use during the first therapy session and to the patient interface in use during the second therapy session;
the step of checking for the difference in behaviour is performed by the respiratory pressure therapy device;
the method comprises transmitting the first vent flow rate data and the second vent flow rate data to a server, and the step of checking for the difference in behaviour is performed by the server;
the step of acquiring or receiving the first vent flow rate data comprises receiving the first vent flow rate data and the step of acquiring or receiving the second vent flow rate data comprises receiving the second vent flow rate data, wherein the steps of receiving the first vent flow rate data and receiving the second vent flow rate data are performed by a server, and the step of checking for the difference in behaviour is performed by the server.

### 3.2.2 Identifying a New Patient Interface Based on AAV Movement

Another aspect of the present technology comprises a method for identifying that a patient interface component comprising an anti-asphyxia valve (AAV) is an unused patient interface component, the method comprising:

acquiring or receiving vent flow rate data during a therapy session, the vent flow rate data representing estimated flow rates of gas to atmosphere including through an AAV of a patient interface in use during ramping up of interface pressure during the therapy session;

identifying AAV movement, based on the vent flow rate data, the AAV movement comprising the AAV reopening and closing after closing a first time during ramping up of interface pressure during the therapy session.

In examples:
the step of identifying the AAV movement comprises identifying two or more reductions in flow rate to atmosphere in response to increased interface pressure during ramping up of interface pressure during the therapy session, each of the reductions indicating a closure of the first AAV;
the step of identifying the AAV movement comprises identifying a first reduction in flow rate to atmosphere in response to increased interface pressure during ramping up of interface pressure during the therapy session, identifying a subsequent increase in flow rate to atmosphere in response to increased interface pressure and then identifying a second reduction in interface pressure;
   In further examples:
the step of acquiring or receiving the vent flow rate data comprises acquiring the vent flow rate data, wherein the step of acquiring the vent flow rate data is performed by a respiratory pressure therapy device providing a pressurised flow of breathable gas to the patient interface in use during the therapy session;
the step of identifying the AAV movement is performed by the respiratory pressure therapy device;
the method comprises transmitting the vent flow rate data to a server and the step of identifying the AAV movement is performed by the server;
the step of acquiring or receiving the vent flow rate data comprises receiving the vent flow rate data, wherein the step of receiving the vent flow rate data is performed by a server, and the step of identifying the AAV movement is performed by the server.

### 3.3 Detecting Patient Interface Replacement Based on Acoustic Signature

Another aspect of the present technology comprises a method for determining that patient interface replacement has occurred between therapy sessions of treatment of sleep disordered breathing, the method comprising:

acquiring or receiving a first acoustic signature of a first patient interface in use during a first therapy session;

acquiring or receiving a second acoustic signature of a second patient interface in use during a second therapy session after the first therapy session;

identifying, by comparison of the second acoustic signature to the first acoustic signature, an acoustic difference between the first acoustic signature and the second acoustic signature indicating that the second patient interface is not the same patient interface as the first patient interface.

In examples:
the step of acquiring or receiving the first acoustic signature comprises acquiring the first acoustic signature and the step of acquiring or receiving the second acoustic signature comprises acquiring the second acoustic signature, wherein the steps of acquiring the first acoustic signature and acquiring the second acoustic signature are performed by a respiratory pressure therapy device operatively connected to the first patient interface during the first therapy session and operatively connected to the second patient interface during the second therapy session;
the acoustic difference is produced as a result of a physical difference between the first patient interface and the second patient interface at a first location within the first patient interface and at a second location within the second patient interface corresponding to the first location;
the first location is at a connection port of the first patient interface and the second location is at a connection port of the second patient interface;
wherein:
   the first acoustic signature comprises one or more first signal magnitudes of one or more respective detected reflections of a first sound from one or more respective first distances from the respiratory pressure therapy device along an air circuit in use during the first therapy session and into the first patient interface; and
   the second acoustic signature comprises one or more second signal magnitudes of one or more respective detected reflections of a second sound from one or more respective second distances from the respiratory pressure therapy device along an air circuit in use during the second therapy session and into the second patient interface;
the first acoustic signature comprises a plurality of first signal magnitudes corresponding to respective first distances from the respiratory pressure therapy device, and the second acoustic signature comprises a plurality of second signal magnitudes corresponding to respective second distances from the respiratory pressure therapy device;
the step of identifying the acoustic difference is performed by the respiratory pressure therapy device;
the method comprises transmitting the first acoustic signature and the second acoustic signature to a server and the step of identifying the acoustic difference is performed by the server;
wherein:
   the step of acquiring the first acoustic signature comprises:
      emitting a first sound from the respiratory pressure therapy device along an air circuit in use during the first therapy session to the first patient interface; and
      detecting a first reflection of the first sound from a first location within the first patient interface; and
      determining a first signal magnitude of the first reflection; and
   the step of acquiring the second acoustic signature comprises:
      emitting a second sound from the respiratory pressure therapy device along an air circuit in use during the second therapy session to the second patient interface;
      detecting a second reflection of the second sound from a second location within the second patient interface corresponding to the first location within the first patient interface; and
      determining a second signal magnitude of the second reflection.
wherein the step of identifying the acoustic difference comprises identifying a difference between the first signal magnitude and the second signal magnitude;
wherein:
   the step of acquiring the first acoustic signature comprises:
      emitting a first sound from the respiratory pressure therapy device along an air circuit in use during the first therapy session to the first patient interface; and
      detecting a plurality of first reflections of the first sound from a plurality of locations within the first patient interface; and
      determining a plurality of first signal magnitudes each corresponding to a respective one of the first reflections; and
the step of acquiring the second acoustic signature comprises:
   emitting a second sound from the respiratory pressure therapy device along an air circuit in use during the second therapy session to the second patient interface; and
   detecting a plurality of second reflections of the sound from a plurality of locations within the second patient interface corresponding to the plurality of locations within the first patient interface; and
   determining a plurality of second signal magnitudes each corresponding to a respective one of the second reflections;
wherein the step of identifying the acoustic difference comprises identifying one or more differences between the first signal magnitudes and the second signal magnitudes;
wherein the step of identifying the acoustic difference comprises identifying two or more differences, each difference being a difference in signal magnitude between one of the first signal magnitudes and a corresponding one of the second signal magnitudes;
the step of acquiring or receiving the first acoustic signature comprises receiving the first acoustic signature and the step of acquiring or receiving the second acoustic signature comprises receiving the second acoustic signature, wherein the steps of receiving the first acoustic signature and receiving the second acoustic signature are performed by a server and the step of identifying the acoustic difference is performed by the server;

Another aspect of the present technology comprises a method of monitoring for patient interface replacement between therapy sessions of treatment of sleep disordered breathing, the method comprising:
acquiring or receiving a first acoustic signature of a patient interface in use during a first therapy session;
acquiring or receiving a second acoustic signature of a patient interface in use during a second therapy session after the first therapy session;
checking for, by comparison of the second acoustic signature to the first acoustic signature, an acoustic difference between the first acoustic signature and the second acoustic signature.

In examples:
the step of acquiring or receiving the first acoustic signature comprises acquiring the first acoustic signature and the step of acquiring or receiving the second acoustic signature comprises acquiring the second acoustic signature, wherein the steps of acquiring the first acoustic signature and acquiring the second acoustic signature are performed by a respiratory pressure therapy device operatively connected to the patient interface in use during the first therapy session and operatively connected to the patient interface in use during the second therapy session;
wherein:
   the first acoustic signature comprises one or more first signal magnitudes of one or more respective first reflections of a first sound from one or more respective distances from the respiratory pressure therapy device along an air circuit in use during the first therapy session and into the patient interface in use during the first therapy session; and
   the second acoustic signature comprises one or more second signal magnitudes of one or more respective second reflections of a second sound from one or more respective distances from the respiratory pressure therapy device along an air circuit in use during the second therapy session and into the patient interface in use during the second therapy session;
the first acoustic signature comprises a plurality of first signal magnitudes corresponding to respective first distances from the respiratory pressure therapy device, and the second acoustic signature comprises a plurality of second signal magnitudes corresponding to respective second distance from the respiratory pressure therapy device;
the step of checking for the acoustic difference is performed by the respiratory pressure therapy device;
the method comprises transmitting the first acoustic signature and the second acoustic signature to a server and the step of checking for the acoustic difference is performed by the server;
the step of acquiring or receiving the first acoustic signature comprises receiving the first acoustic signature and the step of acquiring or receiving the second acoustic signature comprises receiving the second acoustic signature, wherein the steps of receiving the first acoustic signature and receiving the second acoustic signature are performed by a server and the step of checking for the acoustic difference is performed by the server.
wherein:
   the step of acquiring the first acoustic signature comprises:
      emitting a first sound from the respiratory pressure therapy device along an air circuit in use during the first therapy session to the first patient interface; and
      detecting a first reflection of the first sound from a first location within the first patient interface; and
      determining a first signal magnitude of the first reflection; and the step of acquiring the second acoustic signature comprises:
   emitting a second sound from the respiratory pressure therapy
      device along an air circuit in use during the second therapy session to the second patient interface;
      detecting a second reflection of the second sound from a second location within the second patient interface corresponding to the first location within the first patient interface; and
      determining a second signal magnitude of the second reflection.
wherein the step of checking for the acoustic difference comprises checking for a difference between the first signal magnitude and the second signal magnitude;
wherein:
   the step of acquiring the first acoustic signature comprises:
      emitting a first sound from the respiratory pressure therapy device along an air circuit in use during the first therapy session to the first patient interface; and
      detecting a plurality of first reflections of the first sound from a plurality of locations within the first patient interface; and
      determining a plurality of first signal magnitudes each corresponding to a respective one of the first reflections; and
   the step of acquiring the second acoustic signature comprises:
      emitting a second sound from the respiratory pressure therapy device along an air circuit in use during the second therapy session to the second patient interface; and
      detecting a plurality of second reflections of the sound from a plurality of locations within the second patient interface corresponding to the plurality of locations within the first patient interface; and
      determining a plurality of second signal magnitudes each corresponding to a respective one of the second reflections;
wherein the step of checking for the acoustic difference comprises checking for one or more differences between the first signal magnitudes and the second signal magnitudes;
wherein the step of checking for the acoustic difference comprises checking for two or more differences, each difference being a difference in signal magnitude between one of the first signal magnitudes and a corresponding one of the second signal magnitudes;

### 3.4 Determining Patient Interface Replacement Based on Patient Input

Another aspect of the present technology comprises a method for determining that patient interface replacement has occurred, the method comprising:

receiving an input from the patient regarding a patient interface in use;

determining that the patient interface in use has been entered into use for the first time based on the input.

In examples:
the method comprises querying the patient regarding whether the patient interface in use has been entered into use for the first time;
the step of receiving the input comprises receiving identification information, the identification information indicating that the patient interface in use has been entered into use for the first time;
the identification information is unique to the patient interface in use;
the identification information is received following the patient scanning a QR code on the patient interface in use;

In further examples:
the step of receiving an input is performed by a respiratory pressure therapy device;
the step of determining that the patient interface in use has been entered into use for the first time is performed by the respiratory pressure therapy device;
the step of determining that the patient interface in use has been entered into use for the first time is performed by a server;

In further examples:
the step of receiving an input is performed by a computing device of the patient;
the step of determining that the patient interface in use has been entered into use for the first time is performed by a respiratory pressure therapy device;
the step of determining that the patient interface in use has been entered into use for the first time is performed by the computing device;
the step of determining that the patient interface in use has been entered into use for the first time is performed by a server;

### 3.5 Estimating Age of Patient Interface Based on Date

Another aspect of the present technology comprises a method of estimating age of a patient interface, the method comprising:

determining that a patient has entered a patient interface into use for the first time;

recording a date at which the patient interface is entered into use; and

estimating an age of the patient interface by comparing a current date with the date at which the patient interface was entered into use.

In examples:
the step of determining that the patient has entered the patient interface into use for the first time comprises performing a method described herein for determining that patient interface replacement has occurred;
the step of determining that the patient has entered the patient interface into use for the first time comprises performing a method described herein for determining that a patient interface component comprising a vent has been replaced between therapy sessions of treatment of sleep disordered breathing;
the step of determining that the patient has entered the patient interface into use for the first time comprises performing a method described herein for estimating age of a patient interface component comprising a vent;
the step of determining that the patient has entered the patient interface into use for the first time comprises performing a method described herein for determining that a patient interface component comprising an anti-asphyxia valve (AAV) has been replaced between therapy sessions of treatment of sleep disordered breathing;
the step of determining that the patient has entered the patient interface into use for the first time comprises performing a method described herein for determining that patient interface replacement has occurred between therapy sessions of treatment of sleep disordered breathing;
the step of determining that the patient has entered the patient interface into use for the first time comprises performing a method described herein for determining that patient interface replacement has occurred based on identification of an acoustic difference;
the step of determining that the patient has entered the patient interface into use for the first time comprises performing a method described herein for determining that patient interface replacement has occurred based on patient input;
the step of determining that the patient has entered the patient interface into use for the first time comprises receiving patient interface supply data indicating that the patient has been supplied with a new patient interface; and
the method comprises prompting the patient to replace the patient interface based on the estimated age of the patient interface.

### 3.6 Determining Replacement Required Based on Counter

Another aspect of the present technology comprises a method of determining that a patient interface in use requires replacement, the method comprising:

determining that a patient has entered a patient interface into use for the first time;

accruing a value of a usage counter, the usage counter representing an amount of use of the patient interface;

determining that the patient interface requires replacement based at least partially on the value of the usage counter.

In examples:
the step of determining that the patient has entered the patient interface into use for the first time comprises performing a method described herein for determining that patient interface replacement has occurred;
the step of determining that the patient has entered the patient interface into use for the first time comprises performing a method described herein for determining that a patient interface component comprising a vent has been replaced between therapy sessions of treatment of sleep disordered breathing;
the step of determining that the patient has entered the patient interface into use for the first time comprises performing a method described herein for estimating age of a patient interface component comprising a vent;
the step of determining that the patient has entered the patient interface into use for the first time comprises performing a method described herein for determining that a patient interface component comprising an anti-asphyxia valve (AAV) has been replaced between therapy sessions of treatment of sleep disordered breathing;
the step of determining that the patient has entered the patient interface into use for the first time comprises performing a method described herein for determining that patient interface replacement has occurred between therapy sessions of treatment of sleep disordered breathing;
the step of determining that the patient has entered the patient interface into use for the first time comprises performing a method described herein for determining that patient interface replacement has occurred based on identification of an acoustic difference;
the step of determining that the patient has entered the patient interface into use for the first time comprises performing a method described herein for determining that patient interface replacement has occurred based on patient input;
the step of determining that the patient has entered the patient interface into use for the first time comprises receiving patient interface supply data indicating that the patient has been supplied with a new patient interface;
the method comprises zeroing the value of the usage counter after determining that the patient has entered the patient interface into use;
the usage counter represents a number of days of use of the patient interface;
the usage counter represents a number of usage hours of the patient interface;
the usage counter represents a number of therapy sessions since patient interface replacement occurred;
the step of determining that the patient interface requires replacement comprises comparing the value of the usage counter to a threshold value;
the method comprises accruing a value of a supplementary usage counter, the supplementary usage counter being representative of an amount of use of the patient interface;
the step of determining that the patient interface requires replacement comprises comparing the value of the supplementary usage counter to a supplementary threshold value.

### 3.7 Prompting Patient Interface Replacement

Another aspect of the present technology comprises a method of prompting a patient to replace a patient interface or component thereof, the method comprising:

determining that replacement is required of a patient interface or a component thereof in use by a patient during a therapy session for treatment of sleep disordered breathing;

prompting the patient to replace the patient interface or the component thereof.

In examples:
the step of determining that replacement is required is performed by a respiratory pressure therapy device providing a pressurised flow of breathable gas to the patient interface during the therapy session;
the step of prompting the patient is performed by the respiratory pressure therapy device;
the step of prompting the patient is performed by a computing device operated by the patient;
the step of determining that replacement is required is performed by a server with which a respiratory pressure therapy device providing a pressurised flow of breathable gas to the patient interface during the therapy session is configured to communicate;
the step of prompting the patient is performed by the respiratory pressure therapy device;
the step of prompting the patient is performed by a computing device operated by the patient;

### 3.8 Automatic Patient Interface Replacement

Another aspect of the present technology comprises a method of facilitating replacement of a patient interface or component thereof, the method comprising:

determining that replacement is required of a patient interface or a component thereof;

facilitating replacement of the patient interface or the component thereof.

In examples:
the step of determining that replacement is required is performed by a respiratory pressure therapy device providing a pressurised flow of breathable gas to the patient interface during the therapy session;
the step of facilitating replacement comprises ordering a replacement patient interface or component thereof;
the step of facilitating replacement comprises notifying a third party that replacement of the patient interface is required;
the step of facilitating replacement is performed by a server or a computing device operated by the patient;
the step of determining that replacement is required is performed by a server with which a respiratory pressure therapy device providing a pressurised flow of breathable gas to the patient interface during the therapy session is configured to communicate;
the step of facilitating replacement is performed by the respiratory pressure therapy device;
the step of facilitating replacement is performed by a server or a computing device operated by the patient.

### 3.9 Determining that an HMX is in Use

Another aspect of the present technology comprises a method for determining that a patient interface in use by a patient for treatment of sleep disordered breathing comprises a heat and moisture exchanger (HMX), the method comprising:

acquiring or receiving a first acoustic signature of a first patient interface in use during a therapy session;

determining, based on the first acoustic signature, that the first patient interface comprises an HMX.

In examples:
the step of acquiring or receiving the first acoustic signature comprises acquiring the first acoustic signature and the step of determining that the first patient interface comprises the HMX is performed by a respiratory pressure therapy device operatively connected to the first patient interface during the therapy session;
the first acoustic signature comprises one or more first signal magnitudes of one or more respective detected reflections of a first sound from one or more first distances from the respiratory pressure therapy device along an air circuit in use during the therapy session and into the first patient interface;
the first acoustic signature comprises a plurality of first signal magnitudes corresponding to respective first distances from the respiratory pressure therapy device;
the step of determining that the first patient interface comprises an HMX comprises comparing one of the first signal magnitudes corresponding to an expected distance from the respiratory pressure therapy device of the HMX with a reference signal magnitude;
the reference signal magnitude has a value indicating the presence of an HMX and the step of determining that the first patient interface comprises an HMX comprises identifying that the first signal magnitude corresponding to the expected distance of the HMX is substantially the same as the reference signal magnitude;
the reference signal magnitude has a value indicating the absence of an HMX and the step of determining that the first patient interface comprises an HMX comprises identifying that the first signal magnitude corresponding to the expected distance of the HMX is not substantially equal to the reference signal magnitude;
the method further comprises reminding the patient to replace the HMX;
the method further comprises disabling active humidification of a pressurised flow of breathable gas to the patient interface from the respiratory pressure therapy device;
the method further comprises prompting the patient to disable active humidification;
the step of determining that the first patient interface comprises an HMX further comprises determining that the HMX is saturated;
the method further comprises prompting the patient to remove the HMX;
the method comprises transmitting the first acoustic signature to a server and the step of determining that the first patient interface comprises the HMX is performed by the server;
the method comprises transmitting the first acoustic signature to a computing device operated by the patient and the step of determining that the first patient interface comprises the HMX is performed by the computing device; and/or the step of acquiring or receiving the first acoustic signature comprises receiving the first acoustic signature and is performed by a server, the step of determining that the first patient interface comprises an HMX is performed by the server.

Each example identified above under the various aspects is to be understood to be an alternative or an addition to each of the other examples identified above, unless the context clearly required otherwise.

One form of the present technology comprises a system for performing a method according to one or more of the above aspects and/or examples. The system includes at least one memory having computer readable computer instructions, and at least one processor for executing the computer readable instructions. The computer readable instructions include a method according to one or more of the above aspects and/or examples.

An aspect of certain forms of the present technology is a medical device that is easy to use, e.g. by a person who does not have medical training, by a person who has limited dexterity, vision or by a person with limited experience in using this type of medical device.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

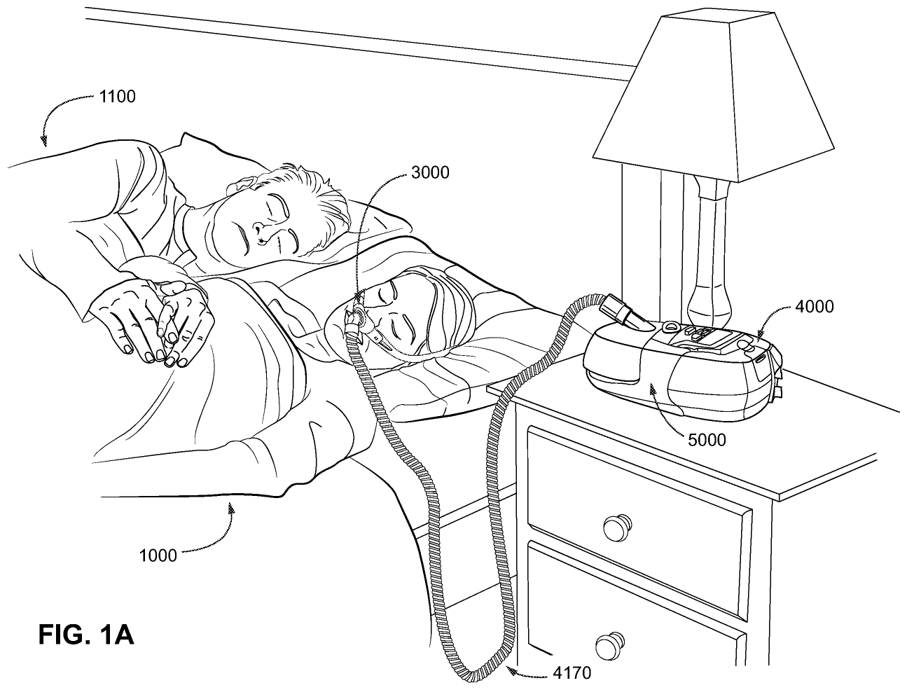

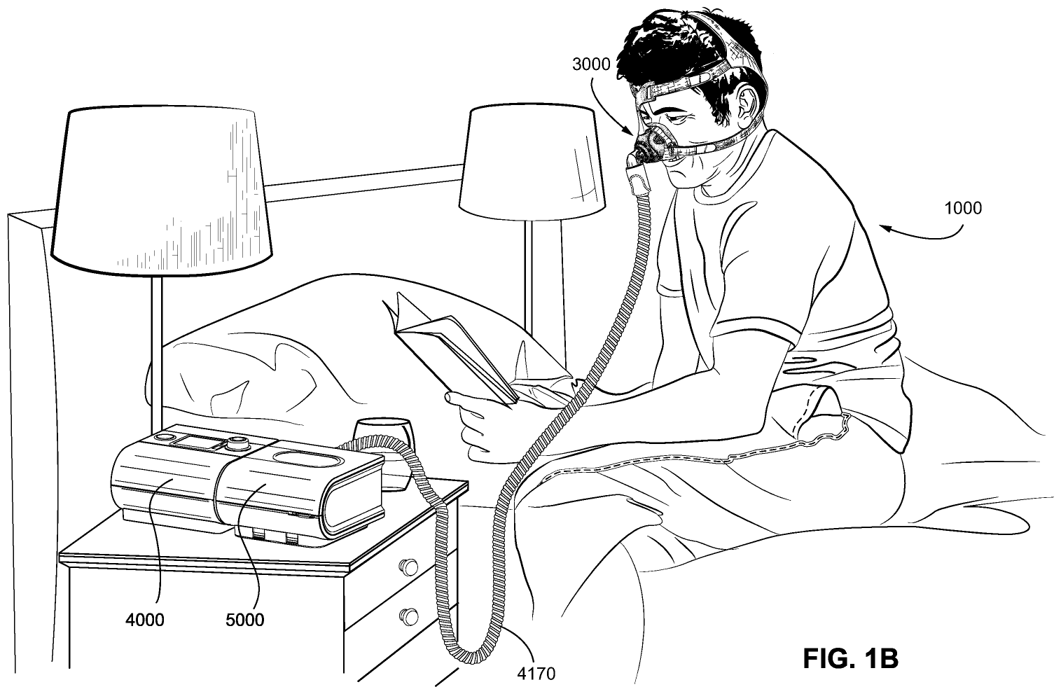

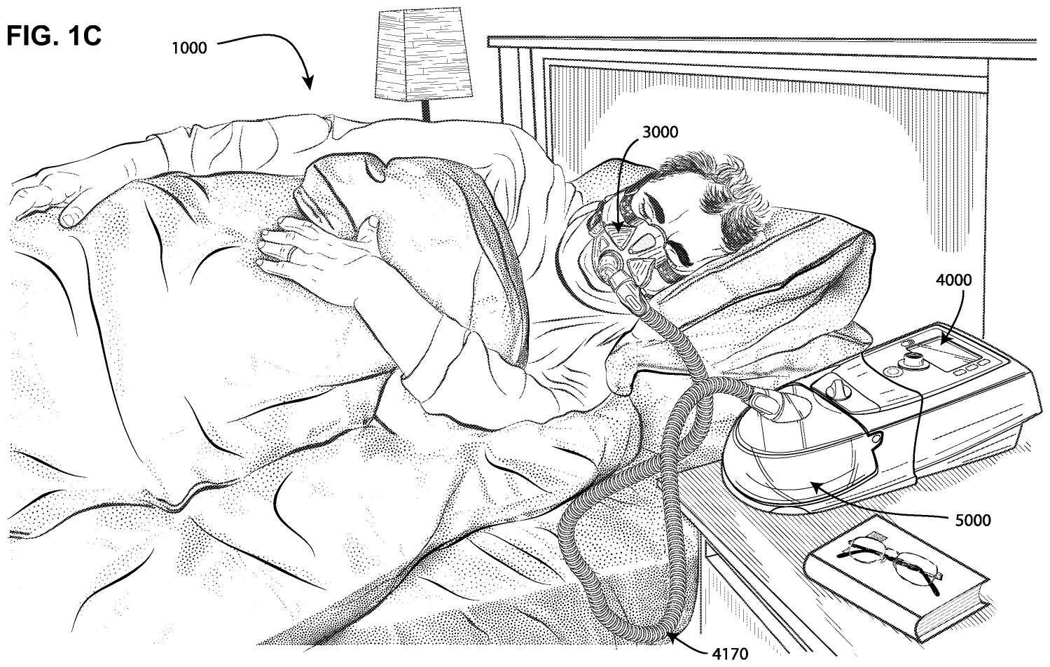

FIG. 1A shows a system including a patient 1000 wearing a patient interface 3000, in the form of nasal pillows, receiving a supply of air at positive pressure from an RPT device 4000. Air from the RPT device 4000 is conditioned in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

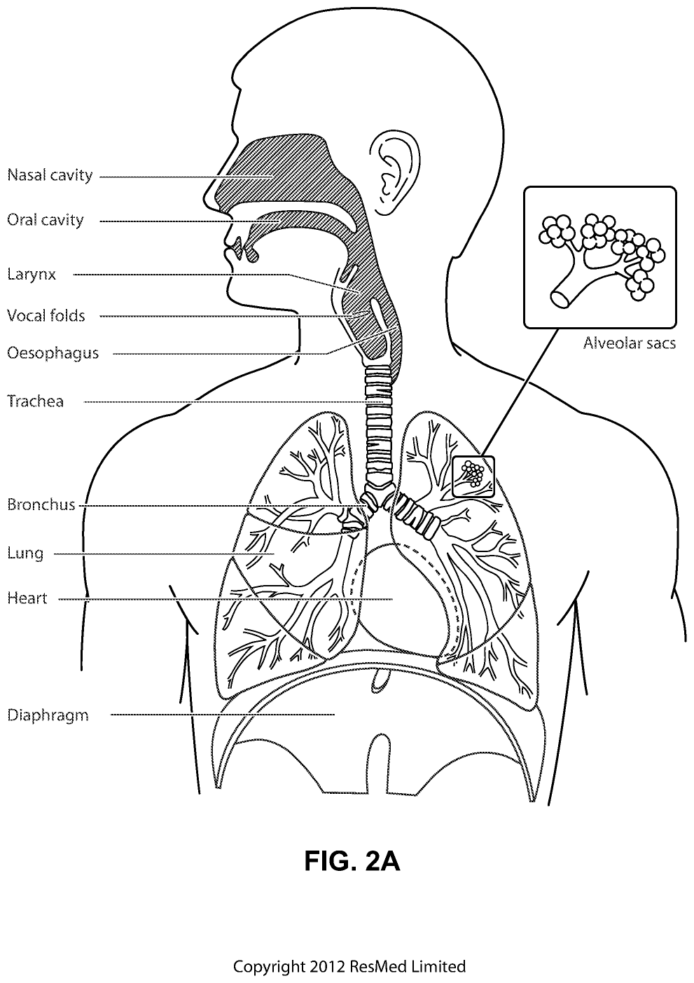

FIG. 2A shows an overview of a human respiratory system including the nasal and oral cavities, the larynx, vocal folds, oesophagus, trachea, bronchus, lung, alveolar sacs, heart and diaphragm.

### 4.3 Patient Interface

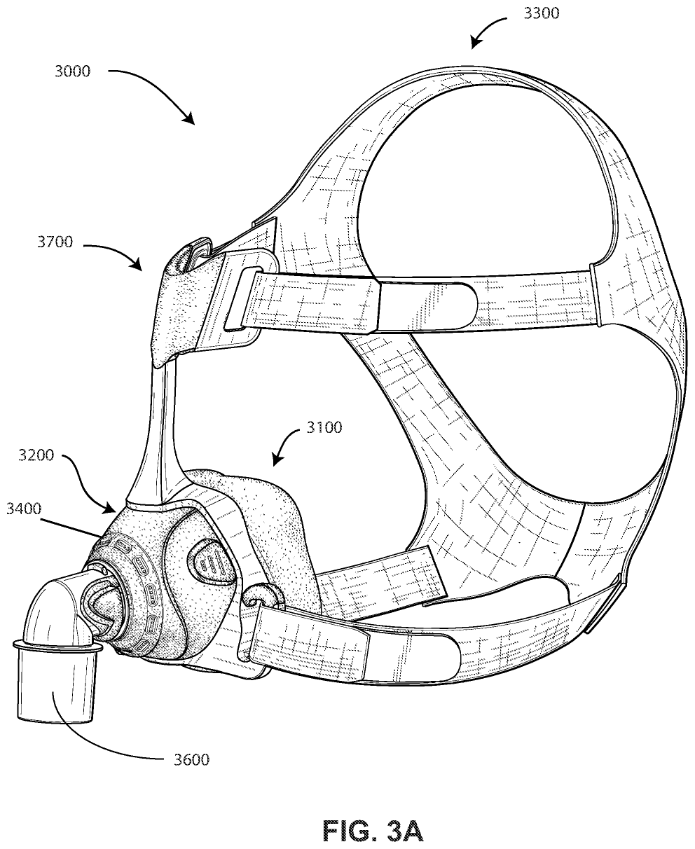

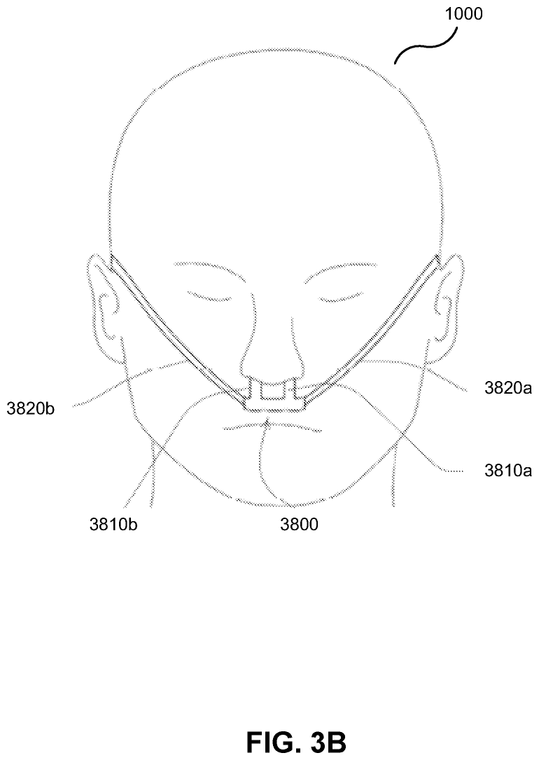

FIG. 3A shows a patient interface in the form of a nasal mask in accordance with one form of the present technology.

FIG. 3B shows a patient interface in the form of a nasal cannula in accordance with one form of the present technology.

### 4.4 RPT Device

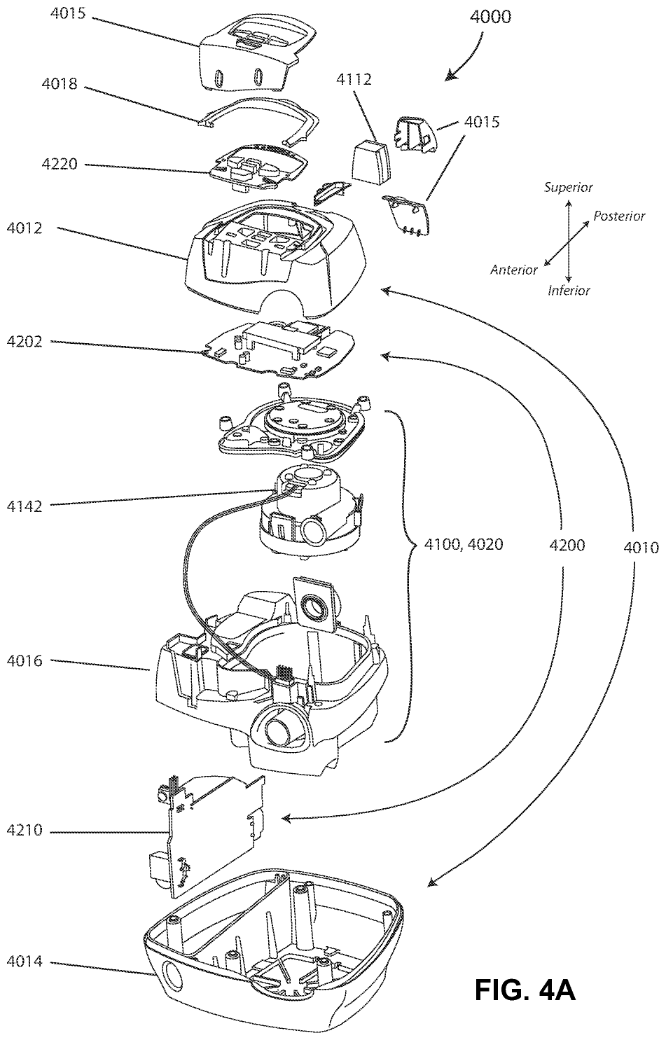

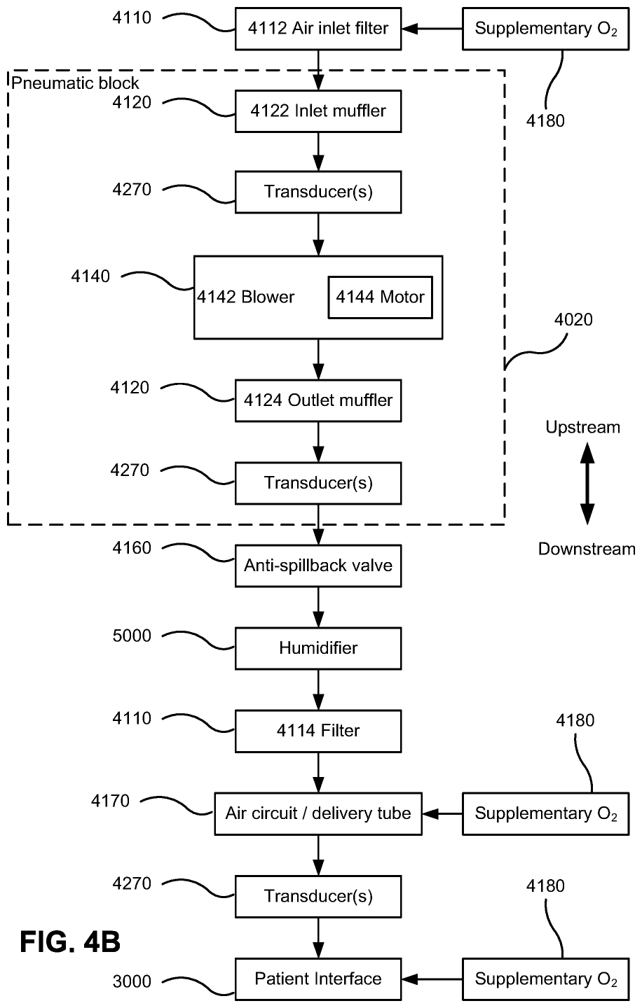

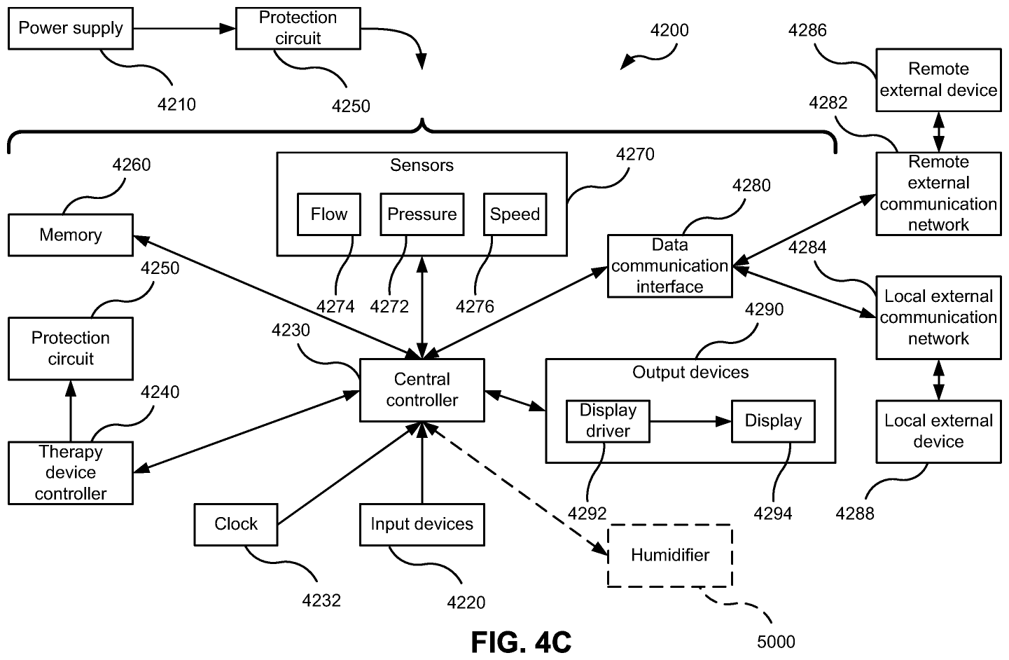

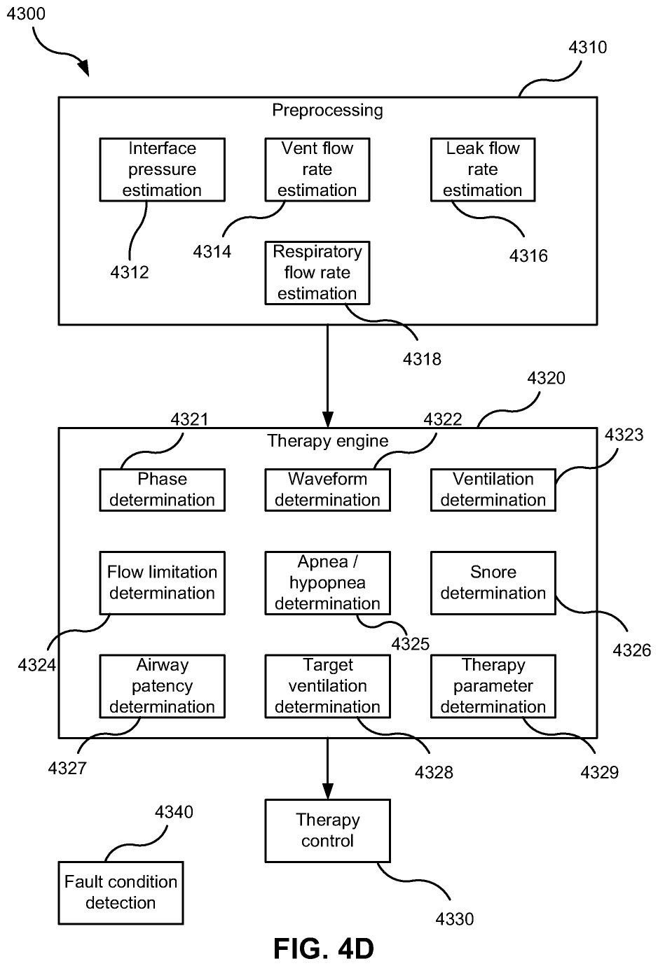

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 4C is a schematic diagram of the electrical components of an RPT device in accordance with one form of the present technology.

FIG. 4D is a schematic diagram of the algorithms implemented in an RPT device in accordance with one form of the present technology.

### 4.5 Humidifier

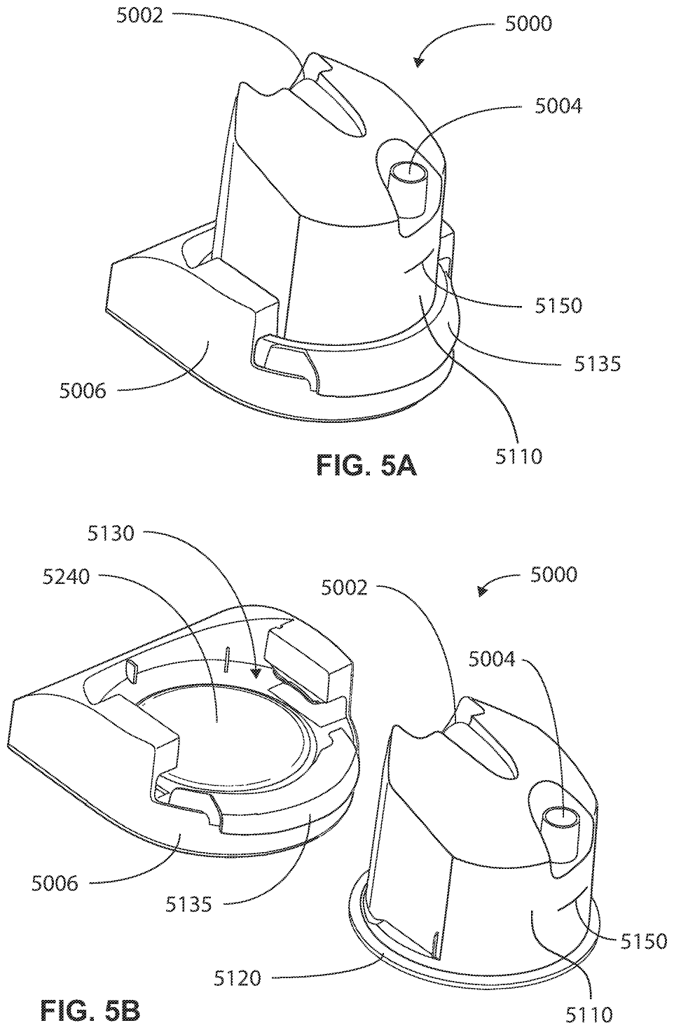

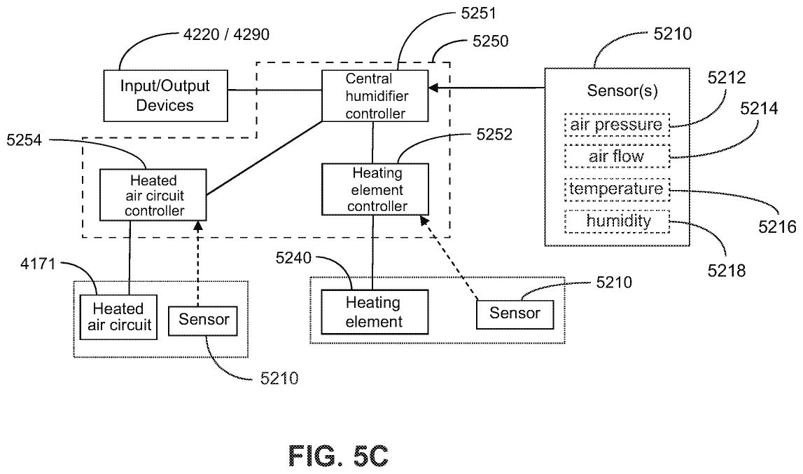

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

FIG. 5C shows a schematic of a humidifier in accordance with one form of the present technology.

### 4.6 Breathing Waveforms

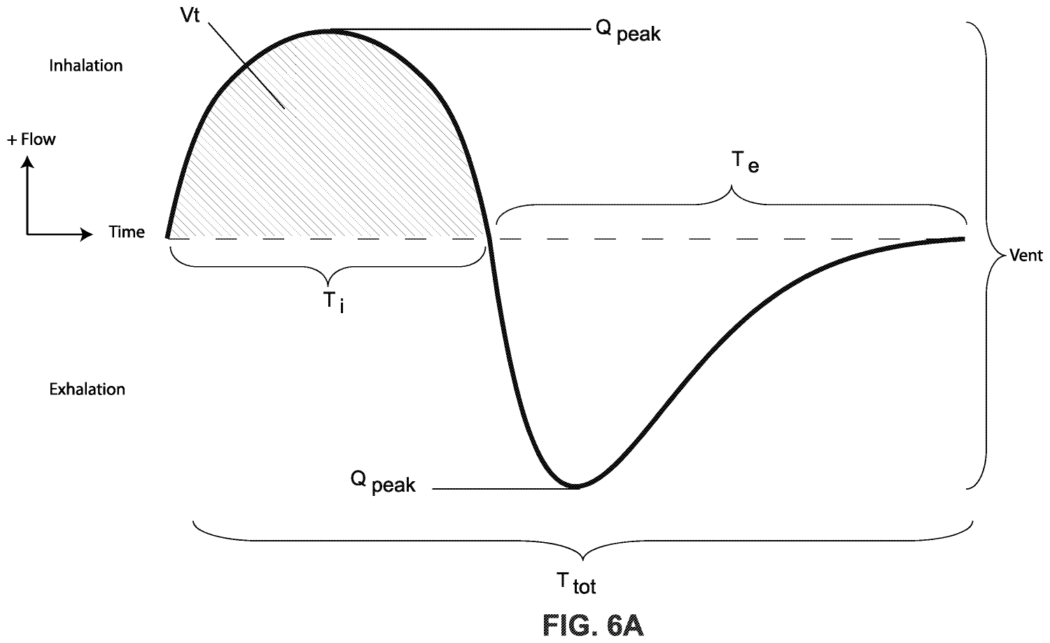

FIG. 6A shows a model typical breath waveform of a person while sleeping.

### 4.7 Computing System and Processes

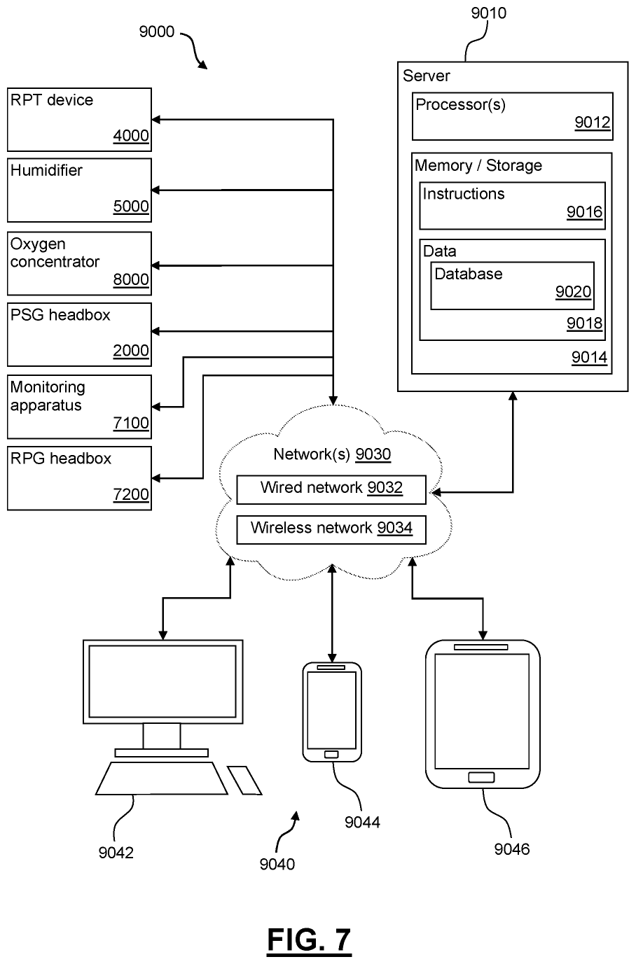

FIG. 7 is a diagram of an example system for performing one or more methods associated with patient interface and component detection, monitoring and/or replacement which includes a computing device.

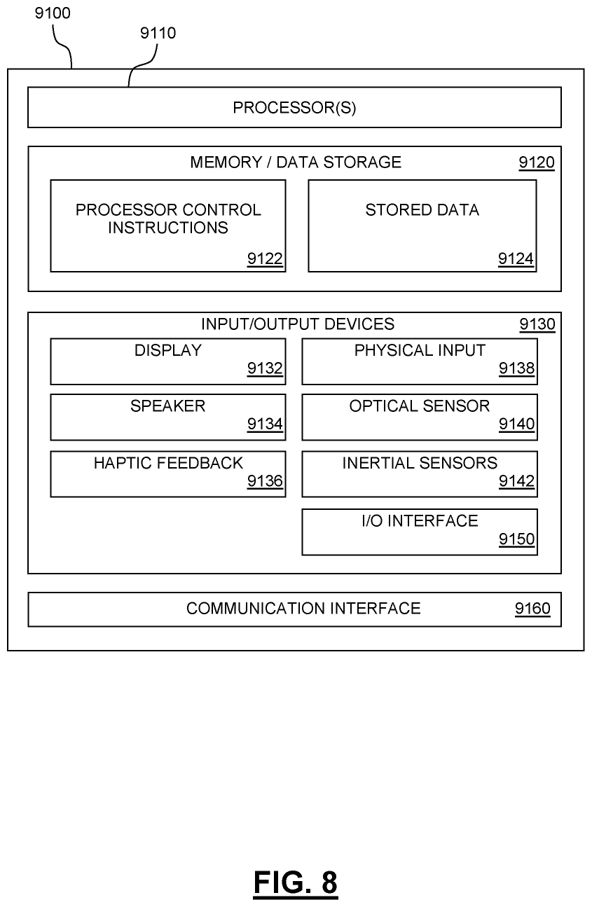

FIG. 8 is a diagram of the components of an example computing device used in performing one or more methods associated with patient interface and component detection, monitoring and/or replacement.

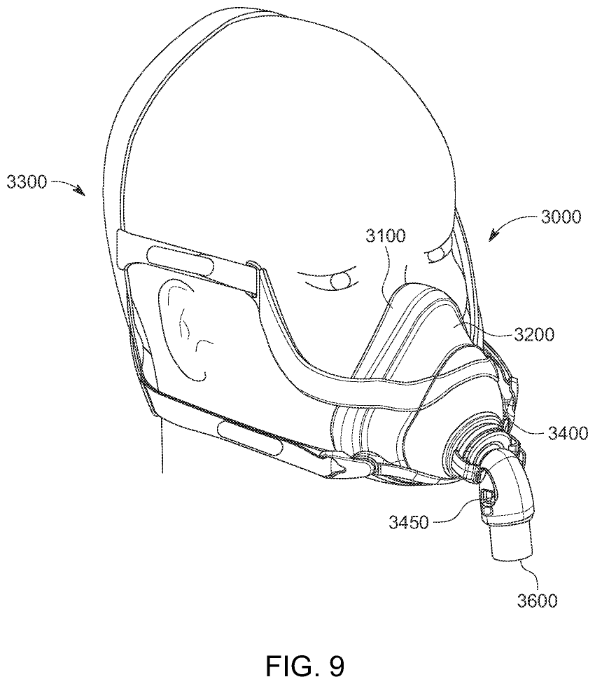

FIG. 9 is an illustration of an example patient interface.

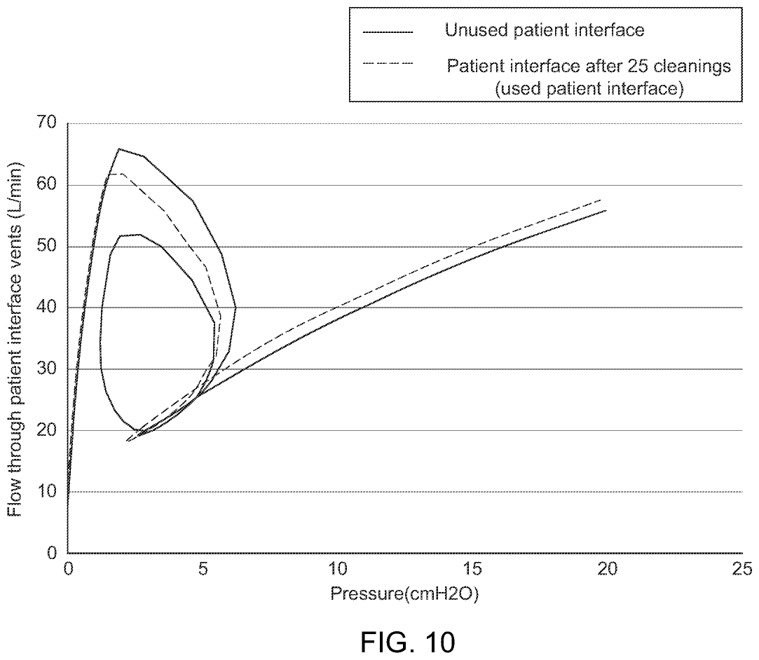

FIG. 10 is a plot of flow through patient interface vents at a range of therapy pressures for an unused patient interface and a used patient interface.

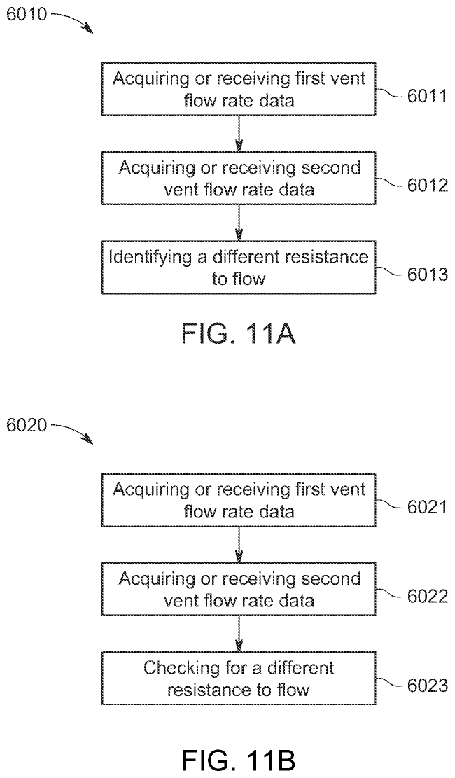

FIGS. 11A and 11B show flow charts of methods according to examples of the present technology.

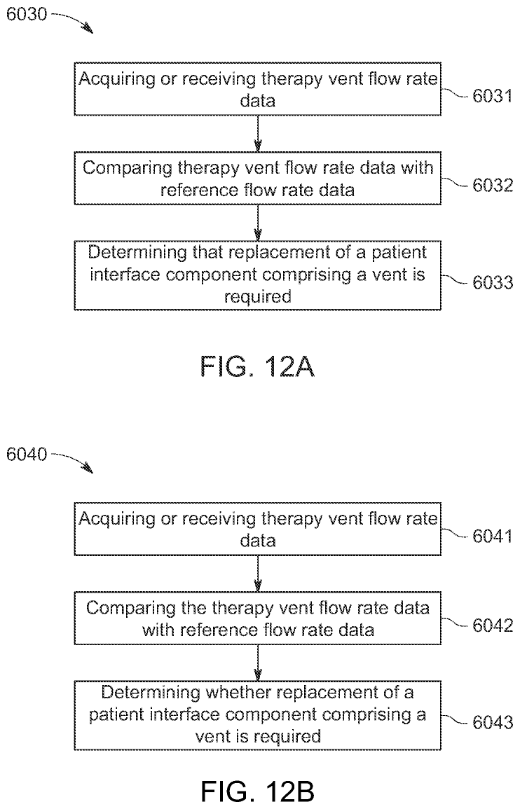

FIGS. 12A and 12B show flow charts of methods according to examples of the present technology.

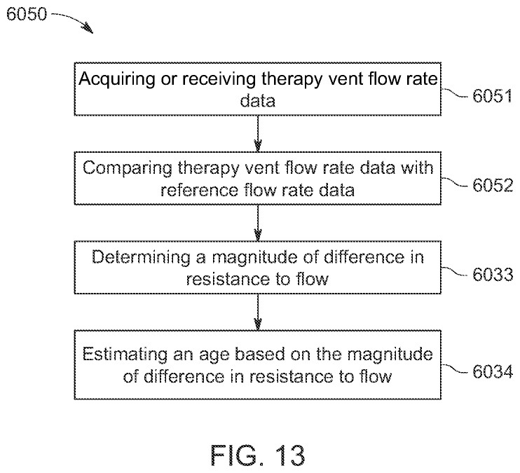

FIG. 13 shows a flow chart of a method according to an example of the present technology.

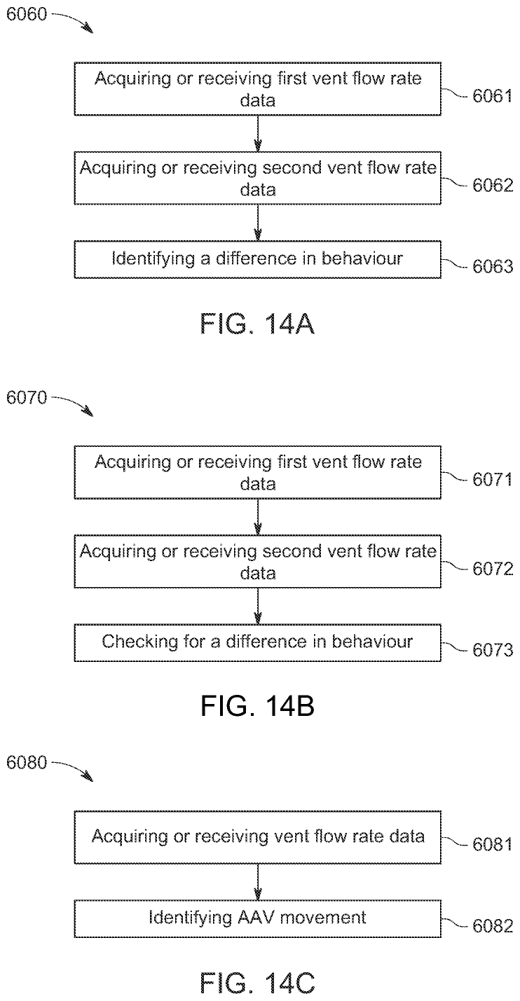

FIGS. 14A-14C show flow charts of methods according to examples of the present technology.

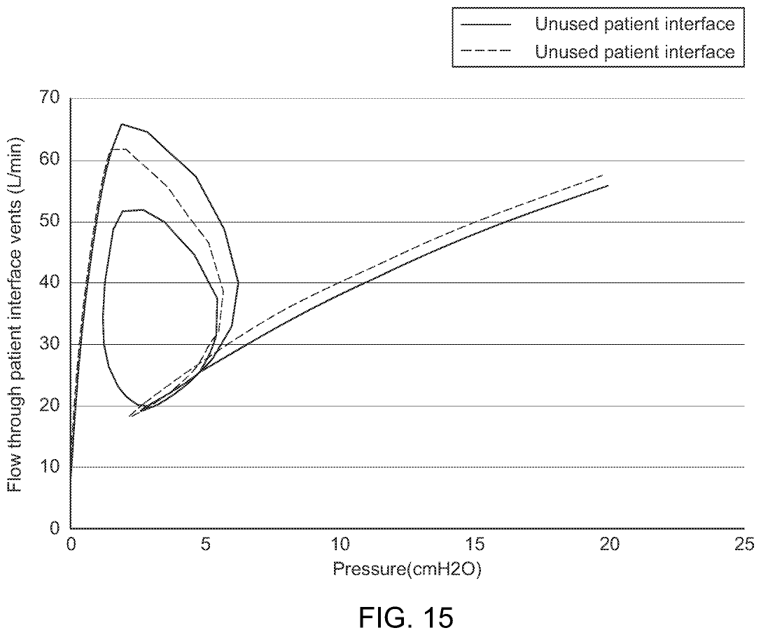

FIG. 15 is a plot of flow through patient interface vents at a range of therapy pressures for an unused patient interface and a used patient interface.

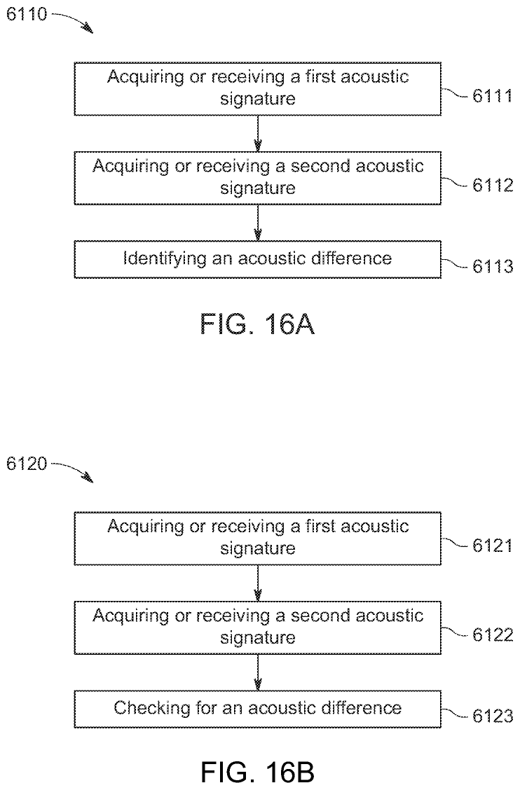

FIGS. 16A and 16B show flow charts of methods according to examples of the present technology.

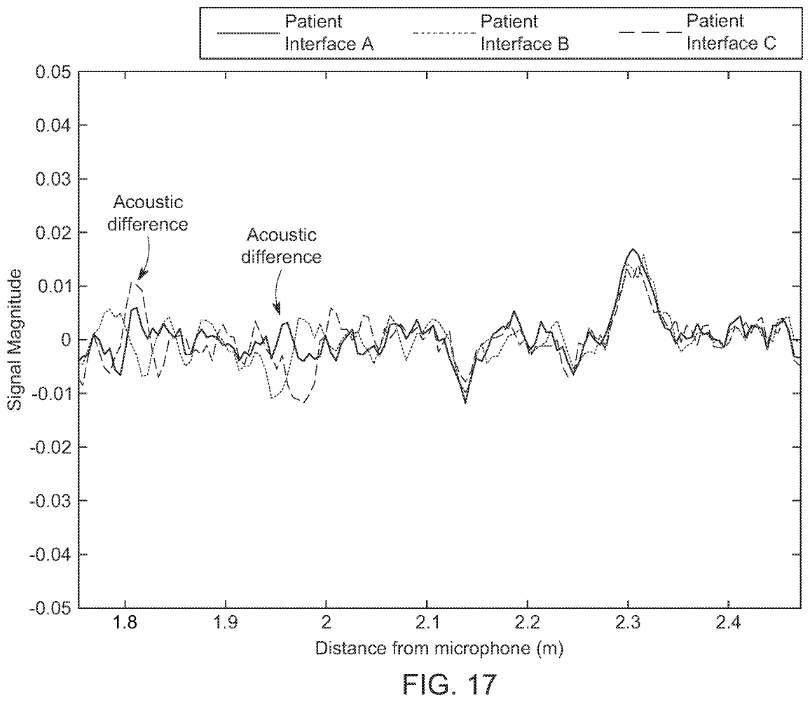

FIG. 17 is a plot showing acoustic signatures for three patient interfaces.

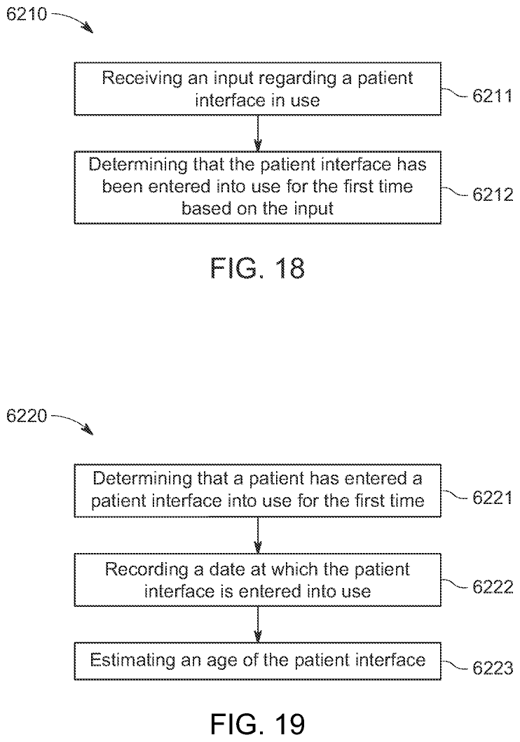

FIGS. 18-23 show flow charts of methods according to examples of the present technology.

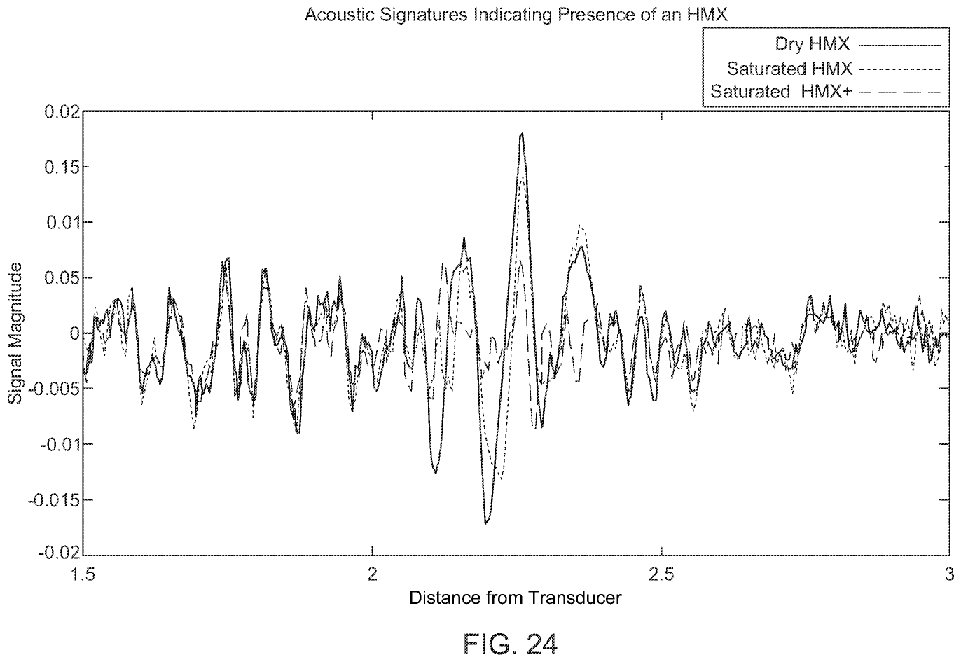

FIG. 24 shows a plot of acoustic signatures for three patient interfaces, each having an HMX.

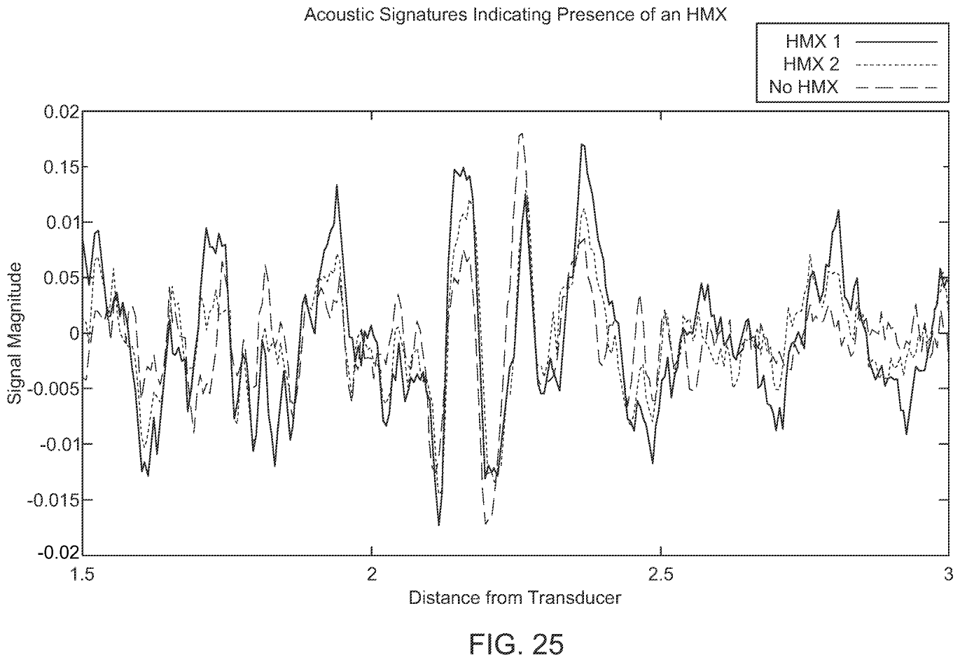

FIG. 25 shows a plot of acoustic signatures for three patient interfaces, two of which comprise an HMX.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may be suitable for delivering any type of respiratory therapy including, but not limited to, continuous positive airway pressure (CPAP) therapy, non-invasive ventilation (NIV), invasive ventilation (IV), high flow therapy (HFT), oxygen concentration and ventilation.

The respiratory therapy system may comprise an RPT device 4000 for supplying a flow of air to the patient 1000 via an air circuit 4170 and a patient interface 3000 or 3800.

### 5.3 Patient Interface

A non-invasive patient interface 3000 in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

The patient interface 3000 in accordance with one form of the present technology is constructed and arranged to be able to provide a supply of air at a positive pressure of at least 6 cmH₂O with respect to ambient.

The patient interface 3000 in accordance with one form of the present technology is constructed and arranged to be able to provide a supply of air at a positive pressure of at least 10 cmH₂O with respect to ambient.

The patient interface 3000 in accordance with one form of the present technology is constructed and arranged to be able to provide a supply of air at a positive pressure of at least 20 cmH₂O with respect to ambient.

### 5.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 5.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 5.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 5.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 5.3.1.5 Forehead Region

In one form, the seal-forming structure that forms a seal in use on a forehead region of the patient's face. In such a form, the plenum chamber may cover the eyes in use.

### 5.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

### 5.3.3 Positioning and Stabilising Structure

The seal-forming structure 3100 of the patient interface 3000 of the present technology may be held in sealing position in use by the positioning and stabilising structure 3300.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. suitable for a small sized head, but not a large sized head.

### 5.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO₂ by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

### 5.3.5 Decoupling Structure(s)

In one form the patient interface 3000 includes at least one decoupling structure, for example, a swivel or a ball and socket.

### 5.3.6 Connection Port

Connection port 3600 allows for connection to the air circuit 4170.

### 5.3.7 Forehead Support

In one form, the patient interface 3000 includes a forehead support 3700.

### 5.3.8 Anti-Asphyxia Valve

In one form, the patient interface 3000 includes an anti-asphyxia valve.

### 5.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 6 cmH₂O, or at least 10cmH₂O, or at least 20 cmH₂O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors 4272 and flow rate sensors 4274. One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 5.4.1 RPT Device Mechanical & Pneumatic Components

An RPT device may comprise one or more of the following components in an integral unit. In an alternative form, one or more of the following components may be located as respective separate units.

### 5.4.1.1 Air Filter(s)

An RPT device in accordance with one form of the present technology may include an air filter 4110, or a plurality of air filters 4110. In one form, an inlet air filter 4112 is located at the beginning of the pneumatic path upstream of a pressure generator 4140. In one form, an outlet air filter 4114, for example an antibacterial filter, is located between an outlet of the pneumatic block 4020 and a patient interface 3000 or 3800.

### 5.4.1.2 Muffler(s)

An RPT device in accordance with one form of the present technology may include a muffler 4120, or a plurality of mufflers 4120. In one form of the present technology, an inlet muffler 4122 is located in the pneumatic path upstream of a pressure generator 4140. In one form of the present technology, an outlet muffler 4124 is located in the pneumatic path between the pressure generator 4140 and a patient interface 3000 or 3800.

### 5.4.1.3 Pressure or Flow Generator

In certain forms of the technology, the RPT device 4000 comprises a pressure generator or flow generator 4140. In one form of the present technology, a pressure or flow generator 4140 for producing a flow, or a supply, of air at positive pressure is a controllable blower 4142. For example the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH₂O to about 20 cmH₂O, or in other forms up to about 30 cmH₂O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 is under the control of the therapy device controller 4240.

In other forms, a pressure generator 4140 may be a piston-driven pump, a pressure regulator connected to a high pressure source (e.g. compressed air reservoir), or a bellows.

### 5.4.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000 or 3800. In examples, the one or more transducers 4270 may comprise a flow rate sensor 4274 (e.g. based on a differential pressure transducer, for example, an SDP600 Series differential pressure transducer from SENSIRION), a pressure sensor 4272 located in fluid communication with the pneumatic path (for example, a transducer from the HONEYWELL ASDX series, or a transducer from the NPA Series from GENERAL ELECTRIC), and/or a motor speed transducer 4276 used to determine a rotational velocity of the motor 4144 and/or the blower 4142 (for example, a speed sensor, such as a Hall effect sensor). In other examples the one or more transducers 4270 may comprise an acoustic sensor (e.g. a microphone) and/or an optical sensor (e.g. a camera or barcode reader).

In one form, a signal from a transducer 4270 may be filtered, such as by low-pass, high-pass or band-pass filtering.

### 5.4.1.4.1 Flow Rate Sensor

A flow rate sensor 4274 in accordance with the present technology may be based on a differential pressure transducer, for example, an SDP600 Series differential pressure transducer from SENSIRION.

In one form, a signal generated by the flow rate sensor 4274 and representing a flow rate is received by the central controller 4230.

### 5.4.1.4.2 Pressure Sensor

A pressure sensor 4272 in Accordance with the Present Technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal generated by the pressure sensor 4272 is received by the central controller 4230.

### 5.4.1.4.3 Motor Speed Transducer

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 5.4.2 RPT Device Electrical Components

### 5.4.2.1 Power Supply

A power supply 4210 may be located internal or external of the external housing 4010 of the RPT device 4000. In one form of the present technology, power supply 4210 provides electrical power to the RPT device 4000 only. In another form of the present technology, power supply 4210 provides electrical power to both RPT device 4000 and humidifier 5000.

### 5.4.2.2 Input Devices

In one form of the present technology, an RPT device 4000 includes one or more input devices 4220 in the form of buttons, switches or dials to allow a person (for example a patient or a clinician) to interact with the device. The buttons, switches or dials may be physical devices, or software devices accessible via a touch screen. The buttons, switches or dials may, in one form, be physically connected to the external housing 4010. In one form of the technology an input device 4220 may take the form of a keypad or keyboard with buttons enabling a user to enter a string of characters, for example a series of alphanumeric characters. The keypad may be formed of physical buttons or regions of a touch screen device visually displayed as buttons, or a combination of such buttons.

In other forms, an input device 4220 may take the form of a remote external device 4286 and/or a local external device 4288 separate, or separable, from the RPT device 4000 and in wireless communication with a data communication interface 4280 of the RPT device 4000 that is in electrical connection to the central controller 4230. Exemplary types of wireless communication between the remote external device 4286 and/or a local external device 4288 and the data communication interface 4280 are stated further below.

In one form of the technology, the input device 4220 is a mobile computing device, for example a mobile phone. The mobile computing device may be operable to communicate directly or indirectly with the central controller 4230, for example via an intermediate communication device and/or via data communication interface 4280. The mobile computing device may be configured to run one or more software applications, or apps, that cause one or more graphical user interfaces (GUIs) to be displayed to a user on a screen of the mobile computing device.

In one form, the input device 4220 may be constructed and arranged to allow a person to select a value and/or a menu option.

In certain forms of the technology, one or more transducers 4270 may operate as input devices 4220 enabling information to be sent to central controller 4230. For example, information may be received acoustically (e.g. via multi-frequency signalling) and this information may be input to the RPT device 4000 by detection of the acoustic signal by an acoustic sensor. In another example, information may be received optically (e.g. via barcode, QR code or coded flashing light) and this information may be input to the RPT device 4000 by detection of the optical signal by an optical sensor. It will be appreciated that the data communication interface 4280 may also comprise one or more transducers 4270 (e.g. antennae) and may act as another input device 4220 by which information can be sent to the central controller 4230.

The input devices 4220 are configured to generate signals representative of information or data input by a user and to send the signals to the central controller 4230. For example, the signals may be electrical signals sent along wired connections to the central controller 4230. Additionally, or alternatively, the signals may be wireless communication signals. In one form of the technology, a keypad generates data representative of a character string entered by a user into the keypad and sends data representative of the character string to the central controller 4230.

### 5.4.2.3 Central Controller

In one form of the present technology, the central controller 4230 is one or a plurality of processors suitable to control an RPT device 4000. Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit. In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and the humidifier 5000. The central controller 4230 may be configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300 expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.4.2.4 Clock

The RPT device 4000 may include a clock 4232 that is connected to the central controller 4230.

### 5.4.2.5 Therapy Device Controller

In one form of the present technology, therapy device controller 4240 is a virtual controller in the form of therapy control module 4330 that forms part of the algorithms 4300 executed by the central controller 4230. In one form of the present technology, therapy device controller 4240 is a dedicated motor control integrated circuit. For example, in one form a MC33035 brushless DC motor controller, manufactured by ONSEMI is used.

### 5.4.2.6 Memory

In accordance with one form of the present technology the RPT device 4000 includes memory 4260, e.g., non-volatile memory. In some forms, memory 4260 may include battery powered static RAM. In some forms, memory 4260 may include volatile RAM. Memory 4260 may be located on the PCBA 4202. Memory 4260 may be in the form of EEPROM, or NAND flash.

Additionally or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms 4300.

### 5.4.2.7 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g. via Ethernet, or optical fibre) or a wireless protocol (e.g. CDMA, GSM, LTE) to connect to the Internet. In one form, local external communication network 4284 utilises one or more communication standards, such as Bluetooth, Near-Field Communication (NFC), or a consumer infrared protocol.

In one form, remote external device 4286 is one or more computers, for example a cluster of networked computers. In one form, remote external device 4286 may be virtual computers, rather than physical computers. In either case, such a remote external device 4286 may be accessible to an appropriately authorised person such as a clinician. The local external device 4288 may be a personal computer, mobile computing device (for example a mobile phone or tablet) or remote control.

### 5.4.2.8 Output Devices Including Optional Display, Alarms

In forms of the technology, the RPT device 4000 includes one or more output devices 4290.

An output device 4290 in accordance with the present technology may take the form of one or more of a visual, audio and haptic unit. A visual display may be a Liquid Crystal Display (LCD) or Light Emitting Diode (LED) display.

In some forms, a display driver 4292 receives as an input the characters, symbols, or images intended for display on the display 4294, and converts them to commands that cause the display 4294 to display those characters, symbols, or images. The display 4294 may be configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol

In forms of the technology the output device 4290 may be comprised as part of a remote external device 4286 and/or a local external device 4288. For example, the output device 4290 may be a display on a mobile computing device (for example a mobile phone or tablet) in wireless communication with the central controller 4230. The mobile computing device may be configured to run one or more software applications, or apps, that cause information to be output on a screen of the mobile computing device.

Data communication interface 4280 may operate as another form of output device 4290 since it may enable information to be output from the RPT device 4000.

### 5.4.3 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 5.4.3.1 Pre-Processing Module

A pre-processing module 4310 in accordance with one form of the present technology receives as an input a signal from a transducer 4270, for example a flow rate sensor 4274 or pressure sensor 4272, and performs one or more process steps to calculate one or more output values that will be used as an input to another module, for example a therapy engine module 4320.

In one form of the present technology, the output values include the interface pressure Pm, the respiratory flow rate Qr, and the leak flow rate Ql.

In various forms of the present technology, the pre-processing module 4310 comprises one or more of the following algorithms: interface pressure estimation 4312, vent flow rate estimation 4314, leak flow rate estimation 4316, and respiratory flow rate estimation 4318.

### 5.4.3.1.1 Interface Pressure Estimation

In one form of the present technology, an interface pressure estimation algorithm 4312 receives as inputs a signal from the pressure sensor 4272 indicative of the pressure in the pneumatic path proximal to an outlet of the pneumatic block (the device pressure Pd) and a signal from the flow rate sensor 4274 representative of the flow rate of the airflow leaving the RPT device 4000 (the device flow rate Qd). The device flow rate Qd, absent any supplementary gas 4180, may be used as the total flow rate Qt. The interface pressure algorithm 4312 estimates the pressure drop ΔP through the air circuit 4170. The dependence of the pressure drop ΔP on the total flow rate Qt may be modelled for the particular air circuit 4170 by a pressure drop characteristic ΔP(Q). The interface pressure estimation algorithm, 4312 then provides as an output an estimated pressure, Pm, in the patient interface 3000 or 3800. The pressure, Pm, in the patient interface 3000 or 3800 may be estimated as the device pressure Pd minus the air circuit pressure drop ΔP.

### 5.4.3.1.2 Vent Flow Rate Estimation

In one form of the present technology, a vent flow rate estimation algorithm 4314 receives as an input an estimated pressure, Pm, in the patient interface 3000 or 3800 from the interface pressure estimation algorithm 4312 and estimates a vent flow rate of air, Qv, from a vent 3400 in a patient interface 3000 or 3800. The dependence of the vent flow rate Qv on the interface pressure Pm for the particular vent 3400 in use may be modelled by a vent characteristic Qv(Pm).

### 5.4.3.1.3 Leak Flow Rate Estimation

In one form of the present technology, a leak flow rate estimation algorithm 4316 receives as an input a total flow rate, Qt, and a vent flow rate Qv, and provides as an output an estimate of the leak flow rate Ql. In one form, the leak flow rate estimation algorithm estimates the leak flow rate Ql by calculating an average of the difference between total flow rate Qt and vent flow rate Qv over a period sufficiently long to include several breathing cycles, e.g. about 10 seconds.

In one form, the leak flow rate estimation algorithm 4316 receives as an input a total flow rate Qt, a vent flow rate Qv, and an estimated pressure, Pm, in the patient interface 3000 or 3800, and provides as an output a leak flow rate Ql, by calculating a leak conductance, and determining a leak flow rate Ql to be a function of leak conductance and pressure, Pm. Leak conductance is calculated as the quotient of low pass filtered non-vent flow rate equal to the difference between total flow rate Qt and vent flow rate Qv, and low pass filtered square root of pressure Pm, where the low pass filter time constant has a value sufficiently long to include several breathing cycles, e.g. about 10 seconds. The leak flow rate Ql may be estimated as the product of leak conductance and a function of pressure, Pm.

### 5.4.3.1.4 Respiratory Flow Rate Estimation

In one form of the present technology, a respiratory flow rate estimation algorithm 4318 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate Ql from the total flow rate Qt.

### 5.4.3.2 Therapy Engine Module

In one form of the present technology, a therapy engine module 4320 receives as inputs one or more of a pressure, Pm, in a patient interface 3000 or 3800, and a respiratory flow rate of air to a patient, Qr, and provides as an output one or more therapy parameters.

In one form of the present technology, a therapy parameter is a treatment pressure Pt.

In one form of the present technology, therapy parameters are one or more of an amplitude of a pressure variation, a base pressure, and a target ventilation.

In various forms, the therapy engine module 4320 comprises one or more of the following algorithms: phase determination 4321, waveform determination 4322, ventilation determination 4323, inspiratory flow limitation determination 4324, apnea/hypopnea determination 4325, snore determination 4326, airway patency determination 4327, target ventilation determination 4328, and therapy parameter determination 4329.

### 5.4.3.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of air whose interface pressure Pm at the patient interface 3000 or 3800 is equal to the treatment pressure Pt.

### 5.4.3.4 Engine and Control Module for Other Operating Parameters

It has been explained that the central controller 4230 may be configured to implement one or more algorithms 4300 for controlling delivery of respiratory therapy, the algorithms being grouped into a pre-processing module 4310, a therapy engine module 4320 and a therapy control module 4330. The central controller 4230 may additionally, or alternatively, be configured to implement one or more algorithms 4300 for controlling other aspects of the operation of the RPT device 4000. The one or more algorithms 4300 for controlling other aspects of the operation of the RPT device 4000 may be grouped into a pre-processing module, an operation engine module and an operation control module.

### 5.4.3.5 Detection of Fault Conditions

In one form of the present technology, the central controller 4230 executes one or more methods 4340 for the detection of fault conditions, for example, power failure (no power, or insufficient power), transducer fault detection, failure to detect the presence of a component, operating parameters outside recommended ranges (e.g. pressure, flow rate, temperature, PaO₂), and failure of a test alarm to generate a detectable alarm signal.

Upon detection of the fault condition, the corresponding algorithm 4340 signals the presence of the fault by one or more of the following: initiation of an audible, visual &/or kinetic (e.g. vibrating) alarm, sending a message to an external device, and logging of the incident.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800. In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used. In some forms, the air circuit 4170 may comprise one or more heating elements configured to heat air in the air circuit, for example to maintain or raise the temperature of the air. The heating element may be in communication with a controller such as a central controller 4230.

### 5.5.1 Supplementary Gas Delivery

In one form of the present technology, supplementary gas, e.g. oxygen, 4180 is delivered to one or more points in the pneumatic path, such as upstream of the pneumatic block 4020, to the air circuit 4170, and/or to the patient interface 3000 or 3800.

### 5.6 Humidifier

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240. In one form, the humidifier 5000 may comprise a humidifier reservoir dock 5130 (as shown in FIG. 5B) configured to receive the humidifier reservoir 5110.

The water reservoir 5110 may be configured to hold, or retain, a volume of liquid (e.g. water) to be evaporated for humidification of the flow of air. The water reservoir 5110 may be configured to hold a predetermined maximum volume of water in order to provide adequate humidification for at least the duration of a respiratory therapy session, such as one evening of sleep. Typically, the reservoir 5110 is configured to hold several hundred millilitres of water, e.g. 300 millilitres (ml), 325 ml, 350 ml or 400 ml. In other forms, the humidifier 5000 may be configured to receive a supply of water from an external water source such as a building's water supply system.

The humidifier 5000 may comprise one or more humidifier transducers (sensors) 5210 instead of, or in addition to, transducers 4270 described above. Humidifier transducers 5210 may include one or more of an air pressure sensor 5212, an air flow rate transducer 5214, a temperature sensor 5216, or a humidity sensor 5218 as shown in FIG. 5C. A humidifier transducer 5210 may produce one or more output signals which may be communicated to a controller such as the central controller 4230 and/or the humidifier controller 5250. In some forms, a humidifier transducer may be located externally to the humidifier 5000 (such as in the air circuit 4170) while communicating the output signal to the controller.

A heating element 5240 may be provided to the humidifier 5000 in some cases to provide a heat input to one or more of the volume of water in the humidifier reservoir 5110 and/or to the flow of air.

According to one arrangement of the present technology, a humidifier 5000 may comprise a humidifier controller 5250 as shown in FIG. 5C. In one form, the humidifier controller 5250 may be a part of the central controller 4230. In another form, the humidifier controller 5250 may be a separate controller, which may be in communication with the central controller 4230.

In one form, the humidifier controller 5250 may receive as inputs measures of properties (such as temperature, humidity, pressure and/or flow rate), for example of the flow of air, the water in the reservoir 5110 and/or the humidifier 5000. The humidifier controller 5250 may also be configured to execute or implement humidifier algorithms and/or deliver one or more output signals.

As shown in FIG. 5C, the humidifier controller 5250 may comprise one or more controllers, such as a central humidifier controller 5251, a heated air circuit controller 5254 configured to control the temperature of a heated air circuit 4171 and/or a heating element controller 5252 configured to control the temperature of a heating element 5240.

### 5.7 Breathing Waveforms

FIG. 6 shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak −0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 5.8 Respiratory Therapy Modes

Various respiratory therapy modes may be implemented by the disclosed respiratory therapy system. Examples of the respiratory therapy modes may include CPAP therapy, bi-level therapy, and high flow therapy.

### 5.9 Computing System and Processes

In forms of the technology, the RPT device 4000 may be part of, or may operate in conjunction with, a system 9000. System 9000 may comprise one or more servers 9010 and one or more computing devices 9040, and may generally be referred to as a computing system 9000. Components of system 9000 may interact with RPT device 4000, for example to control and/or monitor operation of the RPT device 4000. In some examples, system 9000 may enable a person (e.g. a patient, a clinician) to control and/or monitor operation of the RPT device 4000. Controlling and/or monitoring operation of the RPT device 4000 may enable the respiratory therapy provided to the patient 1000 to be controlled and/or monitored.

### 5.9.1 Computing System

FIG. 7 depicts an example system 9000 that may be implemented for use in performing various methods according to the present technology, including methods for determining that patient interface or component replacement has occurred, monitoring for replacement, estimating patient interface age, determining that patient interface replacement is required, prompting a patient to replace a patient interface, detecting presence of an HMX, and the like. The system 9000 may generally include one or more of servers 9010, one or more communication networks 9030, and one or more computing devices 9040. The server 9010 and computing device 9040 may also be in communication with one or more respiratory therapy devices (for example, but not limited to, the RPT device 4000 described in relation to FIG. 4A to FIG. 4D above) via the one or more communication networks 9030.

In the description herein of methods according to the present technology, where reference is made to one or more steps being performed by a server or a computing device, it is to be understood that said steps may be performed by server 9010 or a computing device 9040. Generally, unless the context requires otherwise, any method or method step herein may be performed by either an RPT device 4000, server 9010 and/or computing device 9040. Where different components of the system 9000 perform different method steps, data may be transmitted and/or received via a communication network 9030 as described herein. Where reference is made to a method step being performed by a server 9010 it is to be understood that the method step may alternatively be performed by a computing device 9040. Likewise, where reference is made to a method step being performed by a computing device it is to be understood that the method step may alternatively be performed by a server 9010.

The one or more communication networks 9030 may comprise, for example, the Internet, a local area network, a wide area network and/or a personal area network implemented over wired communication network(s) 9032, wireless communication network(s) 9034, or a combination thereof (for example, a wired network with a wireless link). In one form, local communication networks may utilize one or more communication standards, such as Bluetooth, Near-Field Communication (NFC), or a consumer infrared protocol.

The server 9010 may comprise processing facilities represented by one or more processors 9012, memory 9014, and other components typically present in such computing environments. The processing capabilities of the processor 9012 may be provided, for example, by one or more general-purpose processors, one or more special-purpose processors, or cloud computing services providing access to a shared pool of computing resources configured in accordance with desired characteristics, service models, and deployment models. In the example illustrated the memory 9014 stores information accessible by processor 9012, the information including instructions 9016 that may be executed by the processor 9012 and data 9018 that may be retrieved, manipulated or stored by the processor 9012. The memory 9014 may be of any suitable means known in the art, capable of storing information in a manner accessible by the processor 9012, including a computer readable medium, or other medium that stores data that may be read with the aid of an electronic device. Although the processor 9012 and memory 9014 are illustrated as being within a single unit, it should be appreciated that this is not intended to be limiting, and that the functionality of each as herein described may be performed by multiple processors and memories, that may or may not be remote from each other and the remainder of system 9000.

The instructions 9016 may include any set of instructions suitable for execution by the processor 9012. For example, the instructions 9016 may be stored as computer code on the computer readable medium. The instructions may be stored in any suitable computer language or format. Data 9018 may be retrieved, stored or modified by processor 9012 in accordance with the instructions 9016. The data 9018 may also be formatted in any suitable computer readable format. Again, while the data is illustrated as being contained at a single location, it should be appreciated that this is not intended to be limiting—the data may be stored in multiple memories or locations. The data 9018 may include one or more databases 9020.

In some examples, the server 9010 may communicate one-way with computing device(s) 9040 by providing information to one or more of the computing devices 9040, or vice versa. In other embodiments, server 9010 and computing device(s) 9040 may communicate with each other two-way and may share information and/or processing tasks.

In some examples, the computing device(s) 9040 may include the remote external device 4286 and/or the local external device 4288 described with reference to FIG. 4C above.

### 5.9.2 Computing Devices

The computing device(s) 9040 can be any suitable processing device such as, without limitation, a personal computer such as a desktop or laptop computer 9042, or a mobile computing device such as a smartphone 9044 or tablet 9046. FIG. 8 depicts an exemplary general architecture 9100 of a computing device 9040. Computing device 9040 may include one or more processors 9110. Computing device 9040 may also include memory/data storage 9120, input/output (I/O) devices 9130, and communication interface 9150.

The one or more processors 9110 can include functional components used in the execution of instructions, such as functional components to fetch control instructions from locations such as memory/data storage 9120, decode program instructions, and execute program instructions, and write results of the executed instructions.

Memory/data storage 9120 may be the computing device's internal memory, such as RAM, flash memory or ROM. In some examples, memory/data storage 9120 may also be external memory linked to computing device 9040, such as an SD card, USB flash drive, optical disc, or a remotely located memory (e.g. accessed via a server such as server 9010), for example. In other examples, memory/data storage 9120 can be a combination of external and internal memory.

Memory/data storage 9120 includes processor control instructions 9122 and stored data 9124 that instruct processor 9110 to perform certain tasks, as described herein. As noted above, in examples instructions may be executed by, and data stored in and/or accessed from, resources associated with the server 9010 in communication with the computing device 9040.

In examples, the input/output (I/O) devices 9130 may include one or more displays 9132. In examples, the display 9132 may be a touch sensitive screen allowing for user input in addition to outputting visible information to a user of computing device 9030. In examples, I/O devices may include other output devices, including one or more speakers 9134, and haptic feedback devices 9136. In examples, the input/output (I/O) devices 9130 may include input devices such as physical input devices 9138 (for example, buttons or switches), optical sensors 9140 (for example, one or more imaging devices such as a camera), and inertial sensors 9142 (particularly in examples where the computing device 9040 is a mobile computing device). It will be appreciated that other I/O devices 9130 may be included, or otherwise accessed through an I/O interface 9150 (for example, interfacing with peripheral devices connected to the computing device 9040). A communication interface 9160 enables computing device 9040 to communicate via the one or more networks 9030 (shown in FIG. 7).

### 5.9.3 Computer-Implementable Methods

This specification includes flow diagrams indicating methods implementable, at least in part, by system 9000 in certain forms of the technology. The flow diagrams are representative of example computer readable instructions for implementing the exemplary methods described herein. In examples, the computer readable instructions comprise one or more algorithms for execution by one or more of the processors, for example processors 9012 and/or central controller 4230, described herein. The instructions for performing these functions are, optionally, included in a non-transitory computer readable storage medium, for example memory 9014, or other computer program product configured for execution by one or more processors. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A computer readable storage medium, as used herein, is not to be construed as being transitory signals per se, such as radio waves or other freely propagating electromagnetic waves, electromagnetic waves propagating through a waveguide or other transmission media, or electrical signals transmitted through a wire.

However, persons of ordinary skill in the art will readily appreciate that the entire algorithm and/or parts thereof can alternatively be executed by a device other than a processor and/or embodied in firmware or dedicated hardware in a well-known manner, e.g., it may be implemented by an application specific integrated circuit (ASIC), a programmable logic device (PLD), a field programmable logic device (FPLD), a field programmable gate array (FPGA), discrete logic, etc. For example, any or all of the components can be implemented by software, hardware, and/or firmware. Also, some or all of the instructions represented by the flowcharts may be implemented manually. Further, although the example algorithms are described with reference to the illustrated flowcharts, persons of ordinary skill in the art will readily appreciate that many other methods of implementing the example processor readable instructions may alternatively be used. For example, the order of execution of the blocks may be changed, and/or some of the blocks described may be changed, eliminated, or combined.

As used herein the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor, a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a processor readable medium; or a combination thereof

5.9.4 Patient Interface Replacement and/or Replacement of Patient Interface Components

Some aspects of the present technology, as will be described in more detail below, relate to determining that patient interface replacement is required, checking whether patient interface replacement is required, identifying that a patient interface is a new patient interface, estimating age of a patient interface, estimating an amount of use of a patient interface, and related methods. It is to be understood that methods described in the context of a patient interface may also be applied to individual components of a patient interface, unless the context clearly requires otherwise. For example, a method of determining that a patient interface has been replaced may applied to determine that a particular component of the patient interface has been replaced.

### 5.9.5 Detection of and Monitoring for Replacement of a Patient Interface or a Component Thereof Having a Vent

One example of the present technology is a method 6010 for determining that a patient interface component comprising a vent has been replaced between therapy sessions of treatment of sleep disordered breathing.

FIG. 11A shows a flow chart of the method 6010. The method 6010 will be described here with reference to the patient interface 3000 shown in FIG. 9, which is a full-face mask. It is to be understood that the method 6010 may be applied during use of various types of patient interfaces 3000 having a vent 3400, such as full-face masks, ultracompact full-face masks, nasal masks, nasal cradle masks, pillows masks etc.

In some examples, the method 6010 is used to determine that the vent itself or a particular component of a patient interface comprising the vent has been replaced. In other examples, the method 6010 is used to determine that the whole patient interface has been replaced (since replacement of a patient interface with a vent would include replacement of the vent itself or a patient interface component comprising a vent). For example, if it is not possible due to the configuration of a particular patient interface for only the vent or a component comprising the vent to be replaced, the method 6010 may be used to determine that the patient interface has been replaced. Additionally, in some circumstances in which the method 6010 is applied it may be acceptable to assume that if the vent has been replaced then the entire patient interface has been replaced.

The vent may be a vent which allows a continuous flow of gas to atmosphere/ambient from a plenum chamber of the patient interface throughout the patient's respiratory cycle. This type of vent may be known as a gas washout vent or a bias flow vent, for example. The vent may provide a continuous flow of gas from the interior of the plenum chamber to atmosphere in order to washout exhaled gas from the plenum chamber, preventing excessive CO2 build-up within the plenum chamber. The patient interface 3000 shown in FIG. 9 has a vent 3400 of this type. In other examples the vent may be a different type of vent, such as a vent that opens during exhalation and closes during inhalation.

### 5.9.5.1 Detection Method Steps

In a first method step 6011, the method 6010 may comprise acquiring or receiving first vent flow rate data. The first vent flow rate data may represent one or more estimated first vent flow rates of gas through a vent of a patient interface in use during a first therapy session. In some examples, a therapy session is a night of use of the patient interface. In other examples, a therapy session is a duration of receiving a pressurised flow of air or breathable gas from a flow generator, or a duration of time between turning on and turning off of a flow generator.

In a second method step 6012, the method 6010 may comprise acquiring or receiving second vent flow rate data. The second vent flow rate data may represent one or more second vent flow rates of gas through a vent of a patient interface in use during a second therapy session after the first therapy session.

In a third method step 6013, the method 6010 may comprise identifying a difference in resistance to flow through the first vent than through the second vent indicating that the second vent is not the same vent as the first vent. The identification may be made by comparison of the second vent flow rate data to the first vent flow rate data. A difference in resistance to flow may be used as an indication that the first vent and second vent are not the same vent because a vent in a particular patient interface would not be expected to have a resistance to flow that varies between treatment sessions. In some examples, step 6013 of identifying the difference in resistance to flow may comprise identifying that the difference in resistance to flow is greater than a threshold difference. The threshold difference may be a greater difference in resistance to flow than may be expected to occur between therapy sessions if the vent has not been replaced.

By identification at step 6013 of the method 6010 that the second vent is not the same vent as the first vent, it can be determined that at least the first vent has been replaced between the first therapy session and second therapy session. Therefore, depending on the configuration of the patient interface(s) in use during the first therapy session and second therapy session, it may also be determined that a particular component comprising the first vent, or the entire patient interface in use during the first therapy session, has been replaced between therapy sessions.

With reference to FIG. 9, the illustrated patient interface 3000 comprises a vent 3400. The vent 3400 is formed in a swivel elbow component comprising a connection port 3600 for the patient interface 3000. Performing the method 6010 can therefore determine that between therapy sessions the swivel elbow has been replaced. Furthermore, if circumstances allow for the assumption that the swivel elbow component would not have been replaced on its own (e.g. if the swivel elbow component is not available individually or if there is another reason why only the swivel elbow is not likely to have been replaced), the method 6010 can determine that the patient interface 3000 as whole has been replaced.

### 5.9.5.2 Difference in Resistance to Flow

In some examples of the present technology, the difference in resistance to flow identified at step 6013 may be a greater resistance to flow through the second vent than through the first vent. That is, the second vent may allow less gas to flow to atmosphere at a given therapy pressure than the first vent.

In some examples in which the method 6010 is performed, the patient interface in use by the patient is cleaned periodically, for example by scrubbing the patient interface, including the vent, with a toothbrush. With each cleaning of the patient interface a small amount of material may be removed from the vent, reducing the vent's resistance to flow of gas from the plenum chamber to atmosphere. Over time (e.g. after many cleanings), the vent may have a detectably different resistance to flow than an unused vent of the same type/configuration. When the patient replaces the patient interface or a patient interface component comprising a well-used vent after a first therapy session, the new vent of the patient interface in use during the next therapy session may have a detectably different resistance to flow than the older vent. The method 6010 may detect this difference in resistance to flow to determine that the patient interface or at least the vent has been replaced during therapy sessions.

In other examples of the present technology, the difference in resistance to flow identified at step 6013 of the method 6010 may be a lesser resistance to flow through the second vent than through the first vent. A second vent in use during a second therapy may have a lesser resistance to flow than a first vent in use during a first therapy session, when the second vent has a different configuration than the first vent. For example, the second vent may be of a different type, or the patient interface comprising the second vent may be a different model than the patient interface comprising the first vent or a different variant of the same model.

### 5.9.5.3 Vent Flow Rate Data

Vent flow rate data may represent a plurality of estimated vent flow rates, each corresponding to a respective one of a plurality of therapy pressures (e.g. pressures within the plenum chamber of a patient interface). The pressure and flow rate may be measured by a pressure sensor and a flow rate sensor, respectively, for method 6010 and any other method disclosed herein. Any sensor or arrangement from which pressure can be measured may be considered a pressure sensor. Likewise any sensor or arrangement from which flow rate can be measured may be considered a flow rate sensor.

In some examples of the method 6010, the first vent flow rate data may represent a plurality of estimated first vent flow rates each corresponding to a respective one of a plurality of therapy pressures. Similarly, the second vent flow rate data may represent a plurality of estimated second vent flow rates each corresponding to a respective one of the plurality of therapy pressures. For example, the first and second vent flow rate data may each comprise a set of data points, each data point being a flow rate corresponding to a pressure. The vent flow rate data from such an example may be plotted to show a pressure-flow curve.

FIG. 10 shows a plot of flow rates for a range of therapy pressures. The solid line curve is first vent flow rate data in an example of the present technology, collected during a first therapy session. The broken line curve is second vent flow rate data, collected during a second therapy session. In this example, the patient interface in use during the first therapy session has been cleaned 25 times. The patient interface in use during the second therapy session is an unused (and not yet cleaned) patient interface.

The method 6010 may comprise identifying the difference in resistance to flow at step 6013 by determining that for each one of the plurality of therapy pressures, the corresponding estimate second vent flow rate is different to the corresponding estimated first vent flow rate. Where the difference in resistance to flow is a greater resistance to flow through the second vent than through the first vent, the method 6010 may comprise determining that for each one of the plurality of therapy pressures, the corresponding second vent flow rate is less than the corresponding first vent flow rate. The second vent flow rate may be less than the first vent flow rate if the second vent is a new vent while the first vent is a used vent.

Determining a difference between an estimated second vent flow rate and estimated first vent flow rate may comprise, by way of example only, subtracting the first vent flow rate from the second vent flow rate.

In some examples the step 6013 may comprise identifying the difference in resistance to flow by determining an average difference between respective first and second vent flow rates across the plurality of therapy pressures. Step 6013 may comprise identifying the difference in resistance to flow by determining that the average difference is greater than a threshold difference.

In some examples, step 6013 may comprise identifying the difference in resistance to flow by determining a first impedance for the first patient interface by dividing a measured first pressure by a measured first flow rate and measuring a second impedance for the second patient interface by dividing a measured second pressure by a measured second flow rate, and identifying a difference between the first impedance and second impedance produced by a difference in resistance to flow. The first pressure and second pressure being the same pressure, given the relationship flow rate may not be directly proportional to pressure. Alternatively, step 6013 may comprise determining the first impedance and second impedance based on different pressures and identifying that a difference between the first impedance and second impedance is greater than a threshold impedance difference.

In other examples the flow rate through vents of a first patient interface may be measured during controlled ramping of pressure, and compared with corresponding measurements of flow rate for a second patient interface. Alternatively, the pressure within a plenum chamber of a first patient interface may be measured during a controlled increase in blower RPM, and compared with corresponding measurements of pressure for a second patient interface.

Each one of the plurality of therapy pressures, to which the plurality of estimated first vent flow rates and the plurality of estimated second vent flow rates correspond, may be within the range of 3-30 cmH₂O. In other examples, each one of the therapy pressures may be within the range of 5-20 cmH₂O or 7-20 cmH₂O, for example. As shown in FIG. 10, there may be a more consistent difference between the estimated first vent flow rates and the estimated second vent flow rates above 5 cmH-₂O or 7 cmH₂O. Other effects on vent flow rate may occur around 5 cmH₂O and lower (these will be described below).

In other examples of the method 6010, the first vent flow rate data represents an estimated first vent flow rate (e.g. a single flow rate) corresponding to a predetermined therapy pressure, and the second vent flow rate data represents an estimated second vent flow rate (e.g. a single flow rate) corresponding to the predetermined therapy pressure. The method 6010 may comprise identifying the difference in resistance to flow at step 6013 by determining that the second vent flow rate is different to the first vent flow rate, for example by determining the second vent flow rate is less than or greater than the first vent flow rate. In some examples, the predetermined therapy pressure may be within the range of 3-30 cmH₂O. In other examples the predetermined therapy pressure may be within the range of 5-20 cmH₂O.

To summarise, in some examples of the present technology the first vent flow rate data and second vent flow rate data each comprises multiple vent flow rates each corresponding to one of a plurality of a therapy pressures and the method 6010 comprises determining that at each therapy pressure a second vent flow rate is different to a first vent flow rate, while in other examples the first vent flow rate data and the second vent flow rate data each comprises a single vent flow rate corresponding to a therapy pressure and the method 6010 comprises determining that a single second vent flow rate is different to a single first vent flow rate.

It is to be understood that the first vent flow rate data and second vent flow rate data compared by the method 6010 may each form part of a larger data set. For example, in some examples of the method 6010, step 6011 comprises acquiring or receiving first vent flow rate data by way of receiving a large data set which contains a small data set forming the first vent flow rate data. For example, during step 6011 the method 6010 may comprise receiving data representing hundreds of flow rates at corresponding pressures, a subset of which is the first vent flow rate data (for example less than a hundred or even less than ten flow rates at corresponding pressures). Likewise, during step 6012 the method 6010 may comprise receiving data representing hundreds of flow rates at corresponding pressure, a subset of which is the second vent flow rate data (for example less than a hundred or even less than ten flow rates at corresponding pressures).

To summarise, in a method which compares each flow rate of first vent flow rate data to each flow rate of second vent flow rate data, the first vent flow rate data and second vent flow rate data is the data that is compared, not necessarily the entirety of the data that is initially acquired or received. It may not be necessary to compare each and every pair of corresponding flow rates acquired or received. For example, comparing only one or only a handful of pairs of flow rates (e.g. at pressures of 6, 8, 10 and 12) may provide for a reliable determination that replacement has occurred. Additionally, clearly erroneous data may be ignored. This is to be understood to apply in a corresponding manner to other methods described herein.

### 5.9.5.4 Acquiring or Receiving Vent Flow Rate Data

In some examples of the present technology, step 6011 of the method 6010 comprises acquiring the first vent flow rate data and step 6012 comprises acquiring the second vent flow rate data. The steps 6011 and 6012 of acquiring the first vent flow rate data and acquiring second vent flow rate data may be performed by a respiratory pressure therapy (RPT) device providing a pressurised flow of breathable gas to the patient interface in use during the first therapy session and to the patient interface in use during the second therapy session. That is, during a first therapy session a patient may receive therapy from a patient interface connected to an RPT device 4000, during which time the RPT device 4000 may acquire the first vent flow rate data. During a second therapy session the patient may receive therapy from a different patient interface connected to the RPT device 4000, during which time the RPT device 4000 may acquire the second vent flow rate data.

In some examples, the step 6013 of identifying the difference in resistance is performed by the RPT device 4000. For example, after the RPT device 4000 acquires the first vent flow rate data during a first therapy session and the second vent flow rate data during a second therapy session, the RPT device 4000 may perform step 6013 of the method to identify a difference in resistance to flow through a vent of the patient interface in use during the first therapy session (e.g. a first vent) than through a vent of the patient interface in use during the second therapy session (e.g. a second vent). By identifying a difference in resistance to flow indicating that the second vent is not the same vent at the first vent, the RPT device 4000 can determine that at least the vent in use by the patient was replaced between therapy sessions.

In other examples, the method 6010 may comprise transmitting the first vent flow rate data and the second vent flow rate data to a server 9010. The server 9010 may be remote from the patient. The step 6013 of identifying the difference in resistance may be performed by the server 9010.

In other examples of the present technology, the method 6010 may be performed entirely by a server 9010. For example, step 6011 of the method 6010 may comprise receiving the first vent flow rate data and step 6012 may comprise receiving the second vent flow rate data. The steps 6011 and 6012 of receiving the first vent flow rate data and receiving second vent flow rate data may be performed by a server 9010. The server 9010 may receive the first and second vent flow rate data from an RPT device 4000, for example. The step 6013 of identifying the difference in resistance may then be performed by the server 9010.

Where a server 9010 performs step 6013 of the method 6010, data confirming the identification of the difference in resistance may be transmitted back to the RPT device 4000 that acquired the first vent flow rate data and the second vent flow rate data, or may be transmitted to another party such as health care provider or equipment provider, for example. In some examples, the server 9010 is operated by a health care provider or equipment provider.

### 5.9.5.5 Monitoring Method Steps

Another example of the present technology is a method 6020 for monitoring for replacement of a patient interface component comprising a vent between therapy sessions of treatment of sleep disordered breathing. Method 6020 is related to method 6010 in the sense that, while method 6010 is for detecting that a vent has been replaced, method 6020 is for monitoring for replacement of a vent and may be performed regardless of whether or not replacement actually occurs or is actually detected. Accordingly, method 6020 is described below in the context of the above description of method 6010 and without repetition of every detail that is associated with both methods 6010 and 6020. FIG. 11B shows a flow chart of the method 6020.

The method 6020 may comprise a method step 6021 of acquiring or receiving first vent flow rate data during a first therapy session, the first vent flow rate data representing one or more estimated first vent flow rates of gas through a vent of a patient interface in use during the first therapy session. The method 6020 may also comprise a method step 6022 of acquiring or receiving second vent flow rate data during a second therapy session after the first therapy session, the second vent flow rate data representing one or more estimated second vent flow rates of gas through a vent of a patient interface in use during the second therapy session. Steps 6021 and 6022 of method 6020 may be performed in the same way as steps 6011 and 6012 of method 6010.

In some examples steps 6021 and 6022 are performed at the beginning of therapy sessions. Step 6022 may be performed during at the beginning of the second therapy session so that, if the vent in use during the second therapy session is a new vent (e.g. if the vent and/or the entire patient interface has been replaced prior to the second therapy session), the vent may be clean and the second vent flow rate data may be of high quality. In some examples, step 6021 of acquiring or receiving the first vent flow rate data may be performed multiple times during the first therapy session so that the latest possible first vent flow rate data is available. In other examples, step 6021 may be performed at the beginning of the first therapy session when the vent in use is most likely to be clean, which may provide for high quality first vent flow rate data.

The method 6020 may further comprise a step 6023 of checking for a difference in resistance to flow through the vent of the patient interface in use during the first therapy session than through the vent of the patient interface in use during the second therapy session. While step 6013 of the method 6010 comprises identifying a difference in resistance to flow, step 6023 of the method 6020 comprises checking for a difference in resistance to flow. Step 6023 may be performed regardless of whether there is a difference in resistance to flow detected or not.

The step 6023 of checking for the difference in resistance to flow may be performed by comparison of the second vent flow rate data to the first vent flow rate data.

The difference in resistance to flow checked for at step 6023 may be a greater resistance to flow through the vent of the patient interface in use during the second therapy session than through the vent of the patient interface in use during the first therapy session. As described above in relation to method 6010, a greater resistance to vent flow during the second therapy session than during the first therapy session may indicate that the vent of the patient interface in use during the second therapy session is different from the vent of the patient interface in use during the first therapy session.

The first vent flow rate data and the second vent flow rate data may be the same type of data as described with reference to the method 6010. That is, the first vent flow rate data may represent a plurality of estimated first vent flow rates each corresponding to a respective one of a plurality of therapy pressures, and the second vent flow rate data may represent a plurality of estimated second vent flow rates each corresponding to a respective one of the plurality of therapy pressures. Alternatively, the first vent flow rate data may represent an estimated first vent flow rate corresponding to a predetermined therapy pressure, and the second vent flow rate data may represent an estimated second vent flow rate corresponding to the predetermined therapy pressure. The therapy pressures or pressure, as the case may be, may be within the range of 3-30 cmH₂O, for example within the range of 5-20 cmH₂O or 7-20 cmH₂O.

In examples in which the first and second vent flow rate data comprise a plurality of estimated vent flow rates corresponding to respective therapy pressures, step 6023 of checking for a difference in resistance to flow may comprise checking for a difference, for each one of the plurality of therapy pressures, between the corresponding second vent flow rate and the corresponding first vent flow rate. In examples in which the first and second vent flow rate data each comprises a single estimated vent flow rate corresponding to a predetermined therapy pressure, step 6023 of checking for a difference in resistance to flow may comprise checking for a difference between the second flow rate and the first flow rate. The method 6020 may comprise subtracting the second flow rate from the first flow rate, for example.

The steps 6021 and 6022 of acquiring or receiving the first vent flow rate data and the second vent flow rate data may be performed in the method 6020 in the same way as steps 6011 and 6012 of the method 6010. For example, the first and second vent flow rate data may be acquired by a respiratory pressure therapy device 4000, which may also perform the step 6023 of checking for the difference in resistance to flow. In other examples, the method 6020 may comprise transmitting the first and second vent flow rate data to a server 9010 and the step 6023 of checking for the difference in resistance to flow may be performed by the server 9010. In further examples the first and second vent flow rate data may be received by a server 9010 and the step of checking for the difference in resistance may be performed by the server 9010.

5.9.6 Determining that a Vent Component Requires Replacement Based on Gas Washout Vent Flow

One example of the present technology is a method 6030 of determining that a patient interface component comprising a vent requires replacement. FIG. 12A shows a flow chart of the method 6030.

Like the methods 6010 and 6020, the method 6030 will be described here with reference to the patient interface 3000 shown in FIG. 9, which is a full-face mask. The method 6030 shares details with other methods disclosed herein. The following description will focus on details specific to the method 6030 but it is to be understood that aspects of other methods disclosed herein may be combined, added to or substituted for aspects of method 6030. For example, details of the apparatus in use while the method 6030 is performed will not be repeated and may be found elsewhere herein.

While the method 6030 may be used to determine whether a patient interface component comprising a vent requires replacement, depending on the configuration of the patient interface, different determinations may be made regarding what requires replacement. For example, if the patient interface component comprising the vent is not a replaceable part of the patient interface (e.g. it is not removable or it is not available independently), the method 6030 may effectively determine whether patient interface replacement is required. If the vent is part of a replaceable vent module (e.g. a patient interface component comprising a vent) then the method 6030 may determine that replacement of the replaceable vent module is required.

In a first method step, the method 6030 may comprise a step 6031 of acquiring or receiving therapy vent flow rate data during a therapy session. The therapy vent flow rate data may represent one or more estimated vent flow rates of gas through a vent of a therapy patient interface in use during the therapy session. The therapy patient interface is the patient interface in use by the patient during the therapy session.

In a second method step, the method 6030 may comprise a step 6032 of comparing the therapy vent flow rate data with reference vent flow rate data. The reference vent flow data may represent one or more reference vent flow rates of gas through a reference vent. A reference vent may be a theoretical vent and the reference vent flow rates of gas may be theoretical flow rates through the reference vent. However, in some examples the reference vent flow rates of gas may be determined based on testing the flow rates of gas through a real vent.

In a third method step, the method 6030 may comprise a step 6033 of determining that replacement of a patient interface component comprising the vent of the therapy patient interface is required. The method 6030 may comprise determining that replacement is required based on the comparison of the therapy vent flow rate data to the reference vent flow rate data. That is, the method 6030 may comprise comparing the real-world therapy vent flow rate data (e.g. generated during a real-life therapy session) to theoretical reference vent flow rate data.

### 5.9.6.1 Therapy and Reference Vent Flow Rate Data

In some examples, the therapy vent flow rate data represents a plurality of estimated therapy vent flow rates each corresponding to a respective one of a plurality of therapy pressures. The therapy vent flow rate data may comprise a plurality of data points each representing a flow rate of gas through the vent of the therapy patient interface at a particular therapy pressure. Similarly the reference vent flow rate data may represent a plurality of reference vent flow rates each corresponding to a respective one of the plurality of therapy pressures. The reference vent flow rate data may comprise a plurality of data points each representing a flow rate of gas through the reference vent.

Each one of the plurality of therapy pressures may be within the range of 3-30 cmH₂O. In some examples each of the plurality of therapy pressures may be within the range of 5-20cmH₂O, such as within the range of 7-20cmH₂O, for example.

In other examples, the therapy vent flow rate data represents an estimated therapy vent flow rate corresponding to a predetermined therapy pressure. The therapy vent flow rate data may comprise a single data point representing a flow rate of gas through the vent of the therapy patient interface at a particular therapy pressure. Similarly the reference vent flow rate data may represent a reference vent flow rate corresponding to the predetermined therapy pressure. The reference vent flow rate data may comprise a single data point representing a flow rate of gas through the reference vent at the predetermined therapy pressure.

The predetermined therapy pressure may be within the range of 3-30 cmH₂O, for example within the range of 5-20cmH₂O or 7-20cmH₂O.

### 5.9.6.2 Reference Vent Behaviour

In some examples of the present technology, the reference vent has the behaviour of a vent in an unused patient interface, and in particular examples may be an unused therapy patient interface. That is, the reference vent on which the reference vent flow rate data is based, may be modelled on a vent of an unused (or at least uncleaned) therapy patient interface.

In other examples of the present technology, the reference vent has the behaviour of a vent in a used patient interface, and in particular examples may be a used therapy patient interface. That is, the reference vent on which the reference vent flow rate data is based, may be modelled on the vent of a used therapy patient interface (not new and which has been used by a patient).

As described above, the vent in a used patient interface may have a different behaviour to the vent in an unused patient interface (e.g. of the same type/model). The vent in a used patient interface may allow higher flows of gas, such as a higher flow of gas at a given therapy pressure, than the vent in an unused patient interface. By comparing the behaviour of the vent of a patient interface in use (the therapy patient interface) to a reference vent of a reference patient interface having the behaviour of a known condition (e.g. unused or used), it can be possible to determine condition (e.g. unused or used) of the therapy patient interface.

### 5.9.6.2.1 Unused Patient Interface Reference Vent

In examples in which the reference vent has the behaviour of a vent in an unused patient interface, the step 6033 of determining that replacement is required may comprise, by comparison of the therapy vent flow rate data to the reference vent flow rate data, determining that for each one of the plurality of therapy pressures, the corresponding therapy vent flow rate is substantially equal to or greater than the corresponding reference vent flow rate by a replacement threshold amount. The replacement threshold amount may be a difference between the therapy vent flow rate and the reference vent flow rate that is sufficiently large that it can be determined that replacement of a patient interface component comprising the vent of the therapy patient interface is required. In some specific examples, the replacement threshold amount is 1 L/min, 2 L/min, 3 L/min, 4 L/min or 5 L/min. That is, if the vent of the therapy patient interface allows a flow of gas greater by a predetermined amount than the reference vent allows, the therapy patient interface (or at least a patient interface component comprising the vent of the therapy patient interface) can be determined to require replacement.

In FIG. 9 the solid line curve is reference vent flow rate data representing a plurality of reference vent flow rates of gas through a reference vent having the behaviour of a vent in an unused patient interface. The broken line curve is therapy vent flow rate data representing a plurality of therapy vent flow rates of gas through a vent of a therapy patient interface. In this example of data the therapy patient interface is a used patient interface, having been cleaned 25 times. As illustrated, there is a difference of about 2-3 L/min in greater flow rate through the vent of the therapy patient interface compared to through the reference vent. By determining that there is a difference of this magnitude in flow rate the method 6030 is able to determine that replacement is required. It is to be understood that replacement may be “required” at least based on when it is considered advisable or recommended by a relevant party (e.g. the supplier or a clinician), which may be earlier than when the patient interface ceases to function correctly.

Where the therapy vent flow rate data represents a single estimated therapy vent flow rate corresponding to a predetermined therapy pressure (rather than a plurality of flow rates corresponding to a plurality of pressures), and the reference vent flow rate data represents a single reference vent flow rate corresponding to the predetermined therapy pressure, the step 6033 of determining that replacement is required may comprise determining that the therapy vent flow rate is greater than the reference vent flow rate by a replacement threshold amount. The replacement threshold amount may be as described above.

### 5.9.6.2.2 Used Patient Interface Reference Vent

In examples in which the refence vent has the behaviour of a vent in a used patient interface, the step 6033 of determining that replacement is required may comprise, by comparison of the therapy vent flow rate data to the reference vent flow rate data, determining that for each one of the plurality of therapy pressures, the corresponding therapy vent flow rate is substantially equal to or greater than the corresponding reference vent flow rate. That is, if the vent of the therapy patient interface allows a flow of gas equal to or greater than the reference vent, the therapy patient interface (or at least a patient interface component comprising the vent of the therapy patient interface) can be determined to require replacement.

Where the therapy vent flow rate data represents a single estimated therapy vent flow rate corresponding to a predetermined therapy pressure (rather than a plurality of flow rates and corresponding pressures), and the reference vent flow rate data represents a single reference vent flow rate corresponding to the predetermined therapy pressure, the step 6033 of determining that replacement is required may comprise determining that the therapy vent flow rate is substantially equal to or greater than the reference vent flow rate.

### 5.9.6.3 Acquiring or Receiving Therapy Vent Flow Rate Data

In some examples, step 6031 of the method 6030 comprises acquiring the therapy vent flow rate data (as opposed to receiving it) and may be performed by an RPT device 4000 providing a pressurised flow of breathable gas to the therapy patient interface during a therapy session. In some examples, the step 6032 of comparing the therapy vent flow rate data with reference vent flow rate data and the step 6033 of determining that replacement is required may be performed by the RPT device 4000. In other examples, the method 6030 comprises transmitting the therapy vent flow rate data to a server 9010 and steps 6032 and 6033 performed by the server 9010.

In other examples, step 6031 of the method 6030 comprises receiving the therapy vent flow rate data (as opposed to acquiring it) and may be performed by a server 9010. Additionally, the steps 6032 and step 6033 may be performed by the server 9010.

### 5.9.6.4 Monitoring Method Steps

Another example of the present technology is a method 6040 of checking whether a patient interface component comprising a vent requires replacement. While method 6030 is a method of determining that a patient interface component comprising a vent requires replacement, method 6040 is a method for checking whether or not replacement is required. Method 6040 may be performed regardless of whether or not it is determined that replacement of a patient interface component comprising a vent is required. Accordingly, method 6040 will be described below in the context of the above description of method 6030 without repeating every detail associated with both methods. FIG. 12B shows a flow chart of the method 6040.

The method 6040 comprises a step 6041 of acquiring or receiving therapy vent flow rate data during a therapy session. The therapy vent flow rate may represent one or more estimated vent flow rates of gas through a vent of a therapy patient interface in use during the therapy session. Method 6040 also comprises a step 6042 of comparing the therapy vent flow rate data with reference vent flow rate data, the reference vent flow rate data representing one or more reference flow rates of gas through a reference vent.

The method 6040 also comprises a step 6043 of determining, based on the comparison of the therapy vent flow rate data to the reference vent flow rate data, whether or not replacement of a patient interface component comprising the vent of the therapy patient interface is required.

As described above in relation to the method 6030, the therapy vent flow rate data may represent a plurality of estimated therapy vent flow rates each corresponding to a respective one of a plurality of therapy pressures, and the reference vent flow rate data may represent a plurality of reference vent flow rates each corresponding to a respective one of the plurality of therapy pressures. Each one of the plurality of therapy pressures is within the range of 3-30 cmH₂O, such as within the range of 5-20 cmH₂O.

Also as described above, the reference vent may have the behaviour of a vent in an unused patient interface. In examples in which the therapy vent flow rate data and reference vent flow rate data each represent a plurality of flow rates at a corresponding plurality of therapy pressures, the step 6043 of determining whether or not replacement is required may comprise determining whether, for each one of the plurality of therapy pressures, the corresponding therapy vent flow rate is greater than the corresponding reference vent flow rate by a replacement threshold amount. If so, it is determined that replacement of a patient interface component comprising the vent of the therapy patient interface is required.

Alternatively, the reference vent may have the behaviour of a vent in a used patient interface requiring replacement. Where the therapy vent flow rate data and reference vent flow rate data each represent a plurality of flow rates at a corresponding plurality of therapy pressures, the step 6043 of determining whether or not replacement is required may comprise determining whether, for each one of the plurality of therapy pressures, the corresponding therapy vent flow rate is substantially equal to or greater than the corresponding reference vent flow rate. If so, it is determined that replacement of a patient interface component comprising the vent of the therapy patient interface is required.

In further examples of the method 6040, the therapy vent flow rate data may represent an estimated therapy vent flow rate corresponding to a predetermined therapy pressure, and the reference vent flow rate data represents a reference vent flow rate corresponding to the predetermined therapy pressure. The predetermined therapy pressure may be within the range of 3-30 cmH₂O, for example within the range of 5-20cmH₂O. In such examples, where the reference vent has the behaviour of a vent in an unused patient interface, the step 6043 of determining whether or not replacement is required may comprise determining whether the therapy vent flow rate is greater than the reference vent flow rate by a replacement threshold amount. If so, it is determined that replacement of a patient interface component comprising the vent of the therapy patient interface is required. Alternatively, where the reference vent has the behaviour of a vent in a used patient interface requiring replacement, the step 6043 of determining whether or not replacement is required may comprise determining whether the therapy vent flow rate is substantially equal to or greater than the reference vent flow rate. If so, it is determined that replacement of a patient interface component comprising the vent of the therapy patient interface is required.

Where the step 6041 of acquiring or receiving the therapy vent flow rate data comprises acquiring the therapy vent flow rate data, step 6041 may be performed by a respiratory pressure therapy device 4000 providing a pressurised flow of breathable gas to the therapy patient interface during the therapy session. The step 6042 of comparing the therapy vent flow rate data with reference vent flow rate data and the step 6043 of determining whether or not replacement is required may also be performed by the respiratory pressure therapy device 4000. Alternatively, the method 6040 may comprise transmitting the therapy vent flow rate data to a server 9010 and steps 6042 and 6043 may be performed by the server 9010.

Where the step 6041 of acquiring or receiving the therapy vent flow rate data comprises receiving the therapy vent flow rate data, step 6041 may be performed by a server 9010. Steps 6042 and 6043 may also be performed by the server 9010.

### 5.9.7 Estimating Age of a Patient Interface Based on Gas Washout Vent Flow

In another example of the present technology there is a method 6050 for estimating age of a patient interface component comprising a vent. FIG. 13 shows a flow chart of the method 6050.

Method 6050 comprises a first step 6051 of acquiring or receiving therapy vent flow rate data during a treatment session. The therapy vent flow rate data may represent one or more estimated vent flow rates of gas through a vent of a therapy patient interface in use during the therapy session. Further exemplary details of the therapy vent flow rate data can be found elsewhere herein, for example in relation to the methods 6030 and 6040.

Method 6050 comprises a second step 6052 of comparing the therapy vent flow rate data with reference vent flow rate data. The reference vent flow rate data may represent one or more reference vent flow rates of gas though a reference vent. Further exemplary details of the reference vent flow rate data and reference vent can be found elsewhere herein, for example in relation to the methods 6030 and 6040.

Method 6050 also comprises a step 6053 of determining a magnitude of difference in resistance to flow through the vent of the therapy patient interface than through the reference vent. That is, in a measure of a resistance to flow, a magnitude of the difference between resistance to flow through the vent of the patient interface and resistance to flow through the reference vent.

The method 6050 further comprises a step 6054 of estimating an age of a patient interface component comprising the vent of the therapy patient interface. The estimate may be based on the magnitude of difference in resistance to flow. As described in more detail elsewhere herein, a vent in a used patient interface may have a lesser resistance to flow of gas than a vent in an unused patient interface. Over time, with use (e.g. as the patient interface is cleaned more and more times), resistance to flow of gas through a vent in a patient interface may be reduced. In the method 6050 the resistance to flow through the vent of a patient interface is used to estimate the age of the patient interface.

### 5.9.7.1 Reference Vent is an Unused Vent

In some examples of the method 6050 the reference vent has the behaviour of a vent in an unused patient interface. In some such examples, at step 6053, the method 6050 may comprise determining that the magnitude of difference in resistance to flow is substantially zero and, at step 6054, the method 6050 may comprise estimating that the age of the patient interface component comprising the vent is substantially zero, indicating that the patient interface component comprising the vent is unused. As the reference vent is unused and there is no difference in resistance to flow, it can be inferred that at least the patient interface component comprising the vent in also unused.

In other examples in which the reference vent has the behaviour of a vent in an unused patient interface, at step 6053 the method 6050 may comprise identifying, by comparison of the therapy vent flow rate data to the reference vent flow rate data, a lesser resistance to flow through the vent of the therapy patient interface than through the reference vent. The step 6054 of estimating the age may comprise calculating the age based on an expected rate of change over time of the magnitude of difference in resistance to flow through the vent of the therapy patient interface than through the reference vent. The difference in resistance to flow between the vent of the therapy patient interface and the reference vent indicates that the therapy patient interface (or at least the patient interface component comprising the vent) has been used. With knowledge of a rate at which the resistance to flow through the vent can be expected to change over time with use of the therapy patient interface, an age of the therapy patient interface can be estimated based on how much less resistance to flow there is through the vent of the therapy patient interface in comparison to the unused reference vent.

In some examples of the present technology the reference vent flow rate data may represent flow rate(s) of gas through a reference vent having the behaviour of a vent in a default mask, for example a mask supplied with the RPT device. The reference vent flow rate data may be a factory set default.

### 5.9.7.2 Reference Vent is a Used Vent

In some examples of the method 6050 the reference vent has the behaviour of a vent in a used patient interface having an age at which replacement is required. In some such examples, at step 6053 the method 6050 may comprise determining that the magnitude of difference in resistance to flow is substantially zero and, at step 6054, the method 5060 may comprise estimating that the age of the patient interface component comprising the vent is equal to or greater than the age at which patient interface replacement is required. As the reference vent has the behaviour of a used vent requiring replacement and its resistance to flow is no different to that of the vent under investigation, it can be inferred that the patient interface component comprising the vent is also used and requires replacement.

In other examples of the method 6050 in which the reference vent has the behaviour of a used vent, the method 6050 may comprise at step 6053 identifying, by comparison of the therapy vent flow rate data to the reference vent flow rate data, a greater resistance to flow through the vent of the therapy patient interface than through the reference vent. At step 6054 of estimating the age, the method 6050 may then comprise calculating the age based on an expected rate of change over time of the magnitude of the difference in resistance to flow through the vent of the therapy patient interface than through the reference vent. As a greater resistance to flow through the vent of the therapy patient interface in comparison to the reference vent is detected, it may be determined that the therapy patient interface does not require replacement. However, based on the difference in resistance to flow and an expected rate of change over time of the difference in resistance to flow, the age can be calculated by determining how much “younger” (e.g. newer/less used) the patient interface component comprising the vent is in comparison to the reference vent (having the behaviour of a vent requiring replacement).

### 5.9.7.3 Vent Flow Rate Data

As described in relation to other methods, such as 6030 and 6040, the therapy vent flow rate data may represent a plurality of estimated flow rates corresponding to a respective one of a plurality of therapy pressures. Likewise the reference vent flow rate data may represent a plurality of reference flow rates each corresponding to a respective one of the plurality of therapy pressures.

In such examples, the step 6053 of determining the magnitude of difference in resistance to flow through the vent of the therapy patient interface than through the reference vent may comprise, for each one of the plurality of therapy pressures calculating a difference between the therapy flow rate and the corresponding reference flow rate. The magnitude of difference in resistance to flow may then be represented by an array of differences in flow rate corresponding to the plurality of therapy pressures. Alternatively the magnitude of difference in resistance to flow may be determined by an additional calculation step of calculating an average difference in flow rate across the plurality of therapy pressures. Further still, the magnitude of difference in resistance may be taken to be the greatest of a plurality of differences in flow rate corresponding to the plurality of therapy pressures. Alternatively the magnitude of difference in resistance may taken to be the difference in flow rate at a predetermined one of the plurality of therapy pressures. In yet another example the magnitude of difference in resistance may be taken to be the sum of a plurality of differences in flow rate corresponding to a plurality of therapy pressures.

In other examples, the therapy vent flow rate data may represent an estimated first flow rate corresponding to a predetermined therapy pressure, and the reference vent flow rate data may represent a reference flow rate corresponding to the predetermined therapy pressure. The step of determining the magnitude of difference in resistance to flow comprises calculating a difference between the therapy flow rate and the reference flow rate. For example, the method may comprise subtracting the therapy flow rate from the reference flow rate or performing one or more other calculations to arrive at the difference between the therapy flow rate and reference flow rate.

In some alternative examples, the step of determining the magnitude of difference in resistance to flow may comprise, at one or more predetermined therapy pressures, determining the pressure in the therapy patient interface (e.g. in a plenum chamber of the therapy patient interface) that is produced by a predetermined power output of an RPT device 4000 providing a pressurised flow of gas to the therapy patient interface. The pressure in the therapy patient interface produced by the predetermined power output may then be compared with a reference pressure representing pressure in a reference patient interface (having the reference vent) produced by the predetermined power output. The pressure difference between the pressure in the therapy patient interface and the reference pressure is then a measure of a difference in resistance to flow through the vent of the therapy patient interface than through the reference vent.

Any method or step disclosed herein of determining a magnitude of difference in resistance to flow may also be used to identify the existence of a difference in resistance to flow in any method disclosed here, such as methods 6010 and 6020, and may also be used to compare therapy vent flow rate data with reference vent flow rate data, for example in methods 6030 and 6040. Likewise any method or step disclosed herein of identifying the existence of a difference in resistance to flow is to be understood to be applicable to determining a magnitude of difference in resistance to flow.

### 5.9.7.4 Acquiring or Receiving Therapy Vent Flow Rate Data

In some examples of the present technology, the step 6051 of acquiring or receiving the therapy vent flow rate data may comprise acquiring the therapy vent flow rate data (as opposed to receiving it) and may be performed by a respiratory pressure therapy device 4000 providing a pressurised flow of breathable gas to the therapy patient interface. One or more of the method steps 6052, 6053 and 6054 may also be performed by the respiratory pressure therapy device 4000.

In some examples, the method 6050 may comprise transmitting the therapy vent flow rate data to the server 9010. The server 9010 may then perform steps 6052, 6053 and 6054 of the method. In some examples the respiratory pressure therapy device 4000 may performs steps 6051 and 6052 and then transmit data to a server 9010, which may perform steps 6053 and 6054. In further examples the respiratory pressure therapy device 4000 may perform steps 6051, 6052 and 6053 and then transmit data to a server 9010, which may perform step 6054 of estimating the age.

In other examples of the present technology, the step 6051 of acquiring or receiving the therapy vent flow rate data comprises receiving the therapy vent flow rate data (as opposed to acquiring it) and is performed by a server 9010. One or more of the method steps 6052, 6053 and 6054 may also be performed by the server 9010, for example all of them.

5.9.8 Determining that an AAV Component has been Replaced

In another example of the present technology there is a method 6060 for determining that a patient interface component comprising an anti-asphyxia valve (AAV) has been replaced between therapy sessions of treatment of sleep disordered breathing. FIG. 14A shows a flow chart of the method 6060.

In a first step 6061, the method 6060 comprises acquiring or receiving first vent flow rate data during a first therapy session. The first vent flow rate data may represent estimated flow rates of gas to atmosphere including through a first AAV of a patient interface in use during ramping up of interface pressure during the first therapy session. Interface pressure is to be understood to be the pressure within the patient interface, such as within a plenum chamber of the patient interface from which the patient breathes.

Similarly in a second step 6062, the method 6060 may comprise acquiring or receiving second vent flow rate data during a second therapy session after the first therapy session. The second vent flow rate data may represent estimated flow rates of gas to atmosphere including through a second AAV of a patient interface in use during ramping up of interface pressure during the second therapy session.

The estimated flow rates represented in the first and second vent flow rate data may also include flows to atmosphere via routes other than through the first and second AAVs. For example, while gas may be flowing through each of the first and second AAVs it may also be flowing from the patient interface through a gas washout vent. However, in performing the method 6060, the first and second vent flow rate data represents flow rates of gas flowing at least through an AAV.

In another step 6063, the method 6060 comprises identifying, by comparison of the second vent flow rate data to the first vent flow rate data, a difference in behaviour between the first AAV and the second AAV during ramping up of the interface pressure.

### 5.9.8.1 Difference in Behaviour

In some examples of the present technology, the difference in behaviour identified at step 6063 of the method 6060 may comprise the second AAV closing during ramping up of interface pressure during the second therapy session one or more times more than the first AAV closes during ramping up of interface pressure during the first therapy session. For example, the second AAV may reopen and close after closing a first time during ramping up of interface pressure during the second therapy session, while the first AAV closes only once during ramping up of interface pressure during the first therapy session.

A difference in behaviour between the AAVs of two identical patient interfaces (e.g. of the same model) may be produced by one of the patient interfaces being an unused patient interface and the other being a used patient interface.

FIG. 15 shows vent flow rate data representing flow rates through vents of an unused patient interface (solid line) and through vents of a used patient interface (broken line). The vent flow rate data shown in FIG. 15 comprises a plurality of flow rates at corresponding therapy pressures and is represented as a graph of vent flow rate against therapy pressure. At lower pressures (e.g. below 2 cmH₂O) the vent flow is predominantly through an open AAV of the patient interface and, as AAVs are configured to allow a free flow of air, there is a steep increase in flow rate as therapy pressure increases from 0 cmH₂O to about 2 cmH₂O. As therapy pressure increases above about 2 cmH₂O, the AAV closes, resulting in a drop in vent flow rate as gas is no longer able to flow through the AAV and there is only flow through a gas washout vent, which permits a much lesser flow than the AAV.

In the case of the used patient interface, represented by the broken line in FIG. 15, as pressure within the plenum chamber of the patient interface increases from zero to about 2 cmH₂O there is one sharp increase in vent flow rate as gas begins to flow through the AAV to atmosphere and then a sharp decrease in vent flow as the AAV closes, after which there is a much more gradual increase in vent flow rate as pressure increases, given AAV closes and remains closed, leaving a gas washout vent as the only available vent for gas to flow through. As the gas washout vent allows a much lower flow to atmosphere than the AAV, the increase in vent flow rate with increasing pressure is much less than when the AAV was open.

In the case of the unused patient interface, represented by the solid line in FIG. 15, as pressure within the plenum chamber of the patient interface increases from zero to about 2 cmH₂O there is one sharp increase in vent flow rate as gas begins to flow through the AAV to atmosphere and then a sharp decrease in vent flow as the AAV closes. Until this point during ramping up of pressure, the behaviour of the unused patient interface is similar to the used patient interface. However, as shown by the solid line in FIG. 15, there is a second sharp increase in vent flow followed by a second sharp reduction in vent flow occurring within the 2-5 cmH₂O range of pressures, before a more gradual increase in vent flow rate with increasing pressure. The second sharp increase in vent flow may be caused by the AAV reopening after initially closing. One or more flaps of the AAV may bounce open after initially moving to a closed position. As there is sufficient pressure to close the AAV, the second reduction in flow rate is caused by the AAV closing for a second time. In the case of the vent flow rate data represented by the FIG. 15 plot, the AAV of the unused patient interface closes, reopens and then closes a second time, after which the AAV remains closed and the only vent available for the gas to flow through to atmosphere is a gas washout vent, the result of which is subsequent the gradual increase in vent flow rate with increasing pressure up to about 20 cmH₂O.

It is to be understood that not every patient interface type or model may behave in the same manner as the unused patient interface which produced the data shown in FIG. 15. However, in patient interfaces that do exhibit this behaviour (the closing, reopening and subsequent second closing of the AAV), the method 6060 may be used to determine that at least a patient interface component comprising an AAV has been replaced. In particular examples, the method 6060 may be used to determine that the patient interface as a whole may have been replaced (for example if the patient interface component comprising the AAV is not individually replaceable or available separately).

### 5.9.8.1.1 Identifying Difference in Behaviour

In some examples of the present technology, the step 6063 of identifying the difference in behaviour may comprise identifying a first number of reductions in flow rate to atmosphere in response to increased interface pressure during ramping up of interface pressure during the first therapy session, where each reduction indicates a closure of the first AAV (the first AAV being an AAV of the patient interface in use during the first therapy session). Additionally, step 6063 may comprise identifying a second number of reductions in flow rate to atmosphere in response to increased interface pressure during ramping up of interface pressure during the second therapy session, each of the reductions indicating a closure of the second AAV (being an AAV of the patient interface in use during the second therapy session). The second number of reductions identified may be greater than the first number of reductions and therefore a difference in behaviour between the first AAV and the second AAV.

With reference to FIG. 15, if the used patient interface (the data for which is shown in broken lines) is the patient interface in use during the first therapy session and the unused patient interface (the data for which is shown in a solid line), step 6063 of the method 6060 may comprise identifying that there is one reduction in flow rate during ramping up of pressure during the first therapy session, and two reductions in flow rate during ramping up of pressure during the second therapy session. That is, the method 6060 may comprise identifying that there are a greater number of reductions in flow rate during ramping up of pressure during the second therapy session than during the first therapy session.

In some examples of the method 6060 the first number of reductions identified in step 6063 is only one reduction and the second number of reductions identified is two or more reductions. In particular examples the second number of reductions is two.

The reductions in flow rate may be identified by, for example, calculating differences in flow rates corresponding to two or more therapy pressures between which a reduction in flow rate may be expected to occur and identifying a reduction in flow rate based on one or more negative differences in flow rate with increasing therapy pressure. In another example, the reductions are determined by calculating a rate of change of flow rate with respect to pressure, throughout either an entire therapeutic pressure range (e.g. 0-20 cmH₂O) or a range of pressure at which reductions in flow rate caused by AAV closure is expected to occur (e.g. 0-10cmH₂O or 0-7cmH₂O). The reductions in flow rate may then be identified by the number of times the rate of change of flow rate becomes negative or changes sign. Other suitable methods or calculations of identifying a number of times the flow rate reduces with increasing therapy pressure are to be understood to be possible options for implementing method 6060.

Also visible in FIG. 15 is that, referring to the used patient interface vent flow rate data depicted in broken lines, during and after the reduction in flow rate there is also a reduction in pressure within the plenum chamber of the patient interface. When the AAV of the patient interface closes such that no gas can flow to atmosphere, there is a reduction in pressure (from about 5-6 cmH₂O to about 2 cmH₂O). Referring to the unused patient interface vent flow rate data depicted by solid line, during and after each reduction in flow rate there is a reduction in pressure (from about 6 cmH₂O to about 1-2 cmH₂O). In some examples of the present technology the step 6063 of identifying the difference in behaviour comprises identifying a first number of reductions in pressure within the plenum chamber of a patient interface in use during a first therapy session during ramping up of interface pressure, and identifying a second number of reductions in pressure within the plenum chamber of a patient interface in use during a second therapy session during ramping up of interface pressure, the second number of reductions in pressure being greater than the first number.

### 5.9.8.2 Acquiring or Receiving Vent Flow Rate Data

The method 6060 may be performed by a respiratory pressure therapy device 4000 or may be performed by another device, such as a server 9010 or computing device 9040. An RPT device 4000 may perform each step of the method 6060, a server 9010, computing device 9040 or another device may perform each step or the steps may be performed by a combination of devices. The description, with reference to other methods according to the present technology, of how vent flow rate data may be acquired, transmitted and received by various devices is to be understood to apply to the method 6060 as well.

In some examples of method 6060 the first and second vent flow rate data are acquired by an RPT device 4000. The step 6063 of identifying the difference in behaviour may then performed by the RPT device 4000 or the method may comprise transmitting the first and second vent flow rate data to a server 9010 and step 6063 is performed by the server 9010. In other examples the method is performed entirely by a server 9010, in which case the method 6060 comprises receiving the first and second vent flow rate data at steps 6061 and 6062 and the step 6063 comprises identifying the difference in behaviour. In some examples of the method 6060 the server 9010 may transmit data to the RPT device 4000 regarding the identified difference in behaviour.

### 5.9.8.3 Monitoring Method Steps

Another example of the present technology is a method 6070 for monitoring for replacement of a patient interface component comprising an anti-asphyxia valve (AAV). Method 6070 is related to the method 6060 in the sense that, while method 6060 is a method for detecting that a patient interface component comprising an AAV has been replaced, method 6070 is for monitoring for replacement of a patient interface component comprising an AAV and may be performed regardless of whether or not replacement actually occurs or is actually detected. Accordingly, method 6070 is described below in the context of the above description of method 6060 and without repetition of every detail that is associated with both methods 6060 and 6070. FIG. 14B shows a flow chart of the method 6070.

In a first step 6071 the method 6070 may comprise acquiring or receiving first vent flow rate data during a first therapy session. The first vent flow rate data may represent estimated flow rates of gas to atmosphere including through an AAV of a patient interface in use during the first therapy session and during ramping up of interface pressure. In a second step 6072 the method 6070 may comprise acquiring or receiving second vent flow rate data during a second therapy session after the first therapy session. The second vent flow rate data may represent estimated flow rates of gas to atmosphere including through an AAV of a patient interface in use during the second therapy session and during ramping up of interface pressure.

In a third step 6073, the method 6070 may comprise checking for, by comparison of the second vent flow rate data to the first vent flow rate data, a difference in behaviour between the AAV of the patient interface in use during the first therapy session and the AAV of the patient interface in use during the second therapy session, during ramping up of interface pressure. The difference in behaviour checked for may be any behaviour described with reference to method 6060, for example, or any other behaviour which may indicate that the AAV of the patient interface in use during the second therapy session is not the same AAV as the AAV in use during the first therapy session.

For example, the difference in behaviour checked for may comprise the AAV of the patient interface in use during the second therapy session closing during ramping up of interface pressure one or more times more than the AAV of the patient interface in use during the first therapy session closes during ramping up of interface pressure. The difference in behaviour may be the AAV of the patient interface in use during the second therapy session reopening and closing after closing a first time during ramping up of interface pressure during the second therapy session, while the AAV of the patient interface in use during the first therapy session closes only once during ramping up of interface pressure during the first therapy session.

The step 6073 of checking for the difference in behaviour may comprise identifying a first number of reductions in flow rate to atmosphere in response to increased interface pressure during ramping up of interface pressure during the first therapy session, each of the reductions indicating a closure of the AAV of the patient interface in use during the first therapy session. Step 6073 may further comprise identifying a second number of reductions in flow rate to atmosphere in response to increased interface pressure during ramping up of interface pressure during the second therapy session, each of the reductions indicating a closure of the AAV of the patient interface in use during the second therapy session. The second number of reductions in flow rate may be greater than the first number of reductions, which is a difference in behaviour indicating that the AAV of the patient interface in use during the second therapy session is a new AAV.

In some particular examples, the step 6073 may comprise identifying only one reduction in flow rate to atmosphere in response to increasing interface pressure during ramping up of interface pressure during the first therapy session, the one reduction indicating a closure of the AAV of the patient interface in use during the first therapy session. The step 6073 may further comprise identifying two or more reductions in flow rate to atmosphere in response to increasing interface pressure during ramping up of interface pressure during the second therapy session, each reduction indicating a closure of the AAV of the patient interface in use during the second therapy session. This difference in behaviour indicates that the AAV of the patient interface in use during the second therapy session is a new AAV.

The examples of how the first and second vent flow rate data may be acquired, transmitted and received, as described in relation to method 6060 or other method described herein, are to be understood to be possibilities for method 6070. In particular, an RPT device 4000 may acquire the first and second vent flow rate data at steps 6071 and 6072 and then perform step 6073 or may transmit the first and second vent flow rate data to another device (for example a server 9010 or computing device 9040), which performs step 6073. In other examples the method 6070 may be performed entirely by a device that is not an RPT device, for example a server 9010 or computing device 9040, which receives the first and second vent flow rate data at steps 6071 and 6072 and then performs step 6073.

### 5.9.8.4 Identifying a New Patient Interface Based on AAV Movement

FIG. 14C is a flow chart of a method 6080 for identifying that patient interface component comprising an anti-asphyxia valve (AAV) is an unused patient interface component. The method 6080 comprises a first step 6081 of acquiring or receiving vent flow rate data during a therapy session, the vent flow rate data representing estimated flow rates of gas to atmosphere including through an AAV of a patient interface in use during ramping up of interface pressure during the therapy session. The vent flow rate data may be in the same form as described elsewhere herein (e.g. a plurality of flow rates of gas corresponding to respective interface pressures, in one example). The method 6080 may comprise a second step 6082 of identifying AAV movement, based on the vent flow rate data. The AAV movement identified may comprise the AAV reopening and closing after closing a first time during ramping up of interface pressure during the therapy session. The method 6080 may therefore comprise determining that in response to increasing interface pressure, the AAV (e.g. the flaps thereof) closes a first time, reopens and then closes a second time.

Method 6080 is similar to methods 6060 and 6070 in that flow rates through vents of a patient interface including an AAV are analysed and it is identified that the behaviour of the AAV indicates that the patient interface component comprising the AAV is unused. However, while methods 6060 and 6070 involve comparison of patient interfaces in use in two therapy sessions to identify a difference indicating that the patient interfaces are not the same, method 6080 involves identifying AAV behaviour (the closing, reopening and closing again) which indicates that the AAV component is new, without requiring comparison to a previous therapy session. Advantageously, this form of the present technology may enable detection of a new patient interface (or component thereof), even if not previous vent flow rate data is available.

The step 6082 of identifying the AAV movement may comprise identifying two or more reductions in flow rate to atmosphere in response to increased interface pressure during ramping up of interface pressure during the therapy session, each of the reductions indicating a closure of the first AAV. Detecting the two reductions indicates that the AAV closed twice, meaning it closed a first time, reopened and then closed a second time, which is behaviour exhibited by some AAVs when entered into use for the first time. In particular, the step 6082 of identifying the AAV movement may comprise identifying a first reduction in flow rate to atmosphere in response to increased interface pressure during ramping up of interface pressure during the therapy session, identifying a subsequent increase in flow rate to atmosphere in response to increased interface pressure and then identifying a second reduction in interface pressure.

The step 6081 of acquiring or receiving the vent flow rate data may comprise acquiring the vent flow rate data. In some examples the step of acquiring the vent flow rate data is performed by a respiratory pressure therapy device providing a pressurised flow of breathable gas to the patient interface in use during the therapy session. The step 6082 of identifying the AAV movement may then be performed by the respiratory pressure therapy device.

In other examples, the method 6080 comprises transmitting the vent flow rate data to a server 9010 and the step 6082 of identifying the AAV movement may then be performed by the server 9010.

In further examples, the step 6081 of acquiring or receiving the vent flow rate data may comprise receiving the vent flow rate data. In such examples the step 6081 of receiving the vent flow rate data may be performed by a server 9010. The step 6082 of identifying the AAV movement may also be performed by the server 9010.

### 5.9.9 Determining Patient Interface Replacement Based on Acoustic Signature

Another form of the present technology comprises a method 6110 for determining that patient interface replacement has occurred between therapy sessions of treatment of sleep disordered breathing. FIG. 16A shows a flow chart of the method 6110. Method 6110 may be performed to detect when a patient replaces one patient interface with a new patient interface.

In a first step 6111, the method 6110 may comprise acquiring or receiving a first acoustic signature of a first patient interface in use during a first therapy session. A second step 6112 may comprise acquiring or receiving a second acoustic signature of a second patient interface in use during a second therapy session after the first therapy session.

In some examples, as will be described in more detail herein, each acoustic signature may be a property or properties of a reflection, or multiple reflections, of a sound emitted from an RPT device 4000 (or separate device or module) through an air circuit to the interior of patient interface. For example, the acoustic signature may be data representing a signal magnitude of sound reflected back to an RPT device 4000 from a patient interface and acquired, e.g. by a transducer/microphone. The signal magnitude may vary based on the distance along the air circuit from which it has been reflected. Physical differences between two different patient interfaces may be detected by the method 6110 based on differences in the acoustic signatures, enabling patient interface replacement to be detected by comparison of a latest acoustic signature with a previous acoustic signature. In particular, in a third step 6113, the method 6110 may comprise identifying, by comparison of the second acoustic signature to the first acoustic signature, an acoustic difference between the first acoustic signature and the second acoustic signature indicating that the second patient interface is not the same patient interface as the first patient interface.

An acoustic signature may initially be in the form of or may be derived from data representing a magnitude of sound (as detected by a transducer/microphone) as a function of time. The method may comprise converting the acoustic signature or generating a replacement acoustic signature in the form of data representing a magnitude of sound as a function of distance, for example based on the speed of sound. The method may also comprise a step of normalising the magnitude of the acoustic signature.

The acoustic difference may be produced as a result of a physical difference between the first patient interface and the second patient interface at a first location within the first patient interface and a second location within the second patient interface corresponding to the first location. In some examples, the first location is at a connection port of the first patient interface and the second location is at a connection port of the second patient interface.

The acoustic signature and the acoustic difference will be described in more detail below.

### 5.9.9.1 Acoustic Signatures and Acoustic Difference

The step 6111 of acquiring or receiving the first acoustic signature may comprise acquiring the first acoustic signature (as opposed to receiving it) and the step 6112 of acquiring or receiving the second acoustic signature may comprise acquiring the second acoustic signature (as opposed to receiving it). The first and second acoustic signatures may be acquired by a respiratory pressure therapy device 4000 operatively connected to the first patient interface during the first therapy session and operatively connected to the second patient interface during the second therapy session. Accordingly, an RPT device 4000 may perform steps 6111 and 6112 in some examples.

In some examples, the step 6113 of identifying the acoustic difference is performed by the RPT device 4000. In other examples the method 6110 comprises transmitting the first acoustic signature and the second acoustic signature to a server 9010 and the step 6113 of identifying the acoustic difference is performed by the server 9010.

In other examples, the method 6110 comprises receiving the first acoustic signature (as opposed to acquiring it) and comprises receiving the second acoustic signature (as opposed to acquiring it). The step 6111 of receiving the first acoustic signature and the step 6112 of receiving the second acoustic signature may be performed by a server 9010, computing device 9040 or other device in such examples and the step 6113 of identifying the acoustic difference is performed by the server 9010, computing device 9040 or other device.

FIG. 17 is a plot showing three acoustic signatures for patient interfaces: Patient Interface A, Patient Interface B and Patient Interface C.

### 5.9.9.1.1 Single Signal Magnitude

In examples of the method 6110, the first acoustic signature may comprise one or more first signal magnitudes of one or more respective detected reflections of a first sound from one or more respective first distances from the respiratory pressure therapy device 4000 along an air circuit in use during the first therapy session and into the first patient interface. Likewise the second acoustic signature may comprise one or more second signal magnitudes of one or more respective detected reflections of a second sound from one or more respective second distances from the respiratory pressure therapy device 4000 along an air circuit in use during the second therapy session and into the second patient interface.

For example, in some forms an acoustic signature may be a single signal magnitude of a reflection from a distance from an RPT device 4000. The distance may correspond to the distance of a connection port of the patient interface from the RPT device 4000, for example. Alternatively the distance may correspond to the distance of a particular component of the patient interface, such as the distance to a connector between two parts of an air circuit (e.g. a connector between a conduit connected to the RPT device 4000 and a short tube fluidly connected to a plenum chamber of the patient interface), the distance to an end of the short tube proximate the patient or the distance to a plenum chamber of the patient interface.

In examples in which the acoustic signature is a single signal magnitude, the step 6111 of acquiring the first acoustic signature may comprise emitting a first sound from the respiratory pressure therapy device 4000 along an air circuit in use during the first therapy session to the first patient interface. Step 6111 may further comprise detecting a first reflection of the first sound from a first location within the first patient interface and determining a first signal magnitude of the first reflection. Similarly the step 6112 of acquiring the second acoustic signature may comprise emitting a second sound from the respiratory pressure therapy device 4000 along an air circuit in use during the second therapy session to the second patient interface. Step 6112 may further comprise detecting a second reflection of the second sound from a second location within the second patient interface corresponding to the first location within the first patient interface and determining a second signal magnitude of the second reflection.

The step 6113 of identifying the acoustic difference may then comprise identifying a difference between the first signal magnitude and the second signal magnitude. As one particular example, in performing the method 6110 a first signal magnitude may be determined during use of a patient interface during one night of therapy (a first therapy session) and then a second signal magnitude may be determined during a subsequent night of therapy (a second therapy session). The signal magnitudes may be of reflections of a sound from the location of a connection port of the patient interface in use during each therapy session. A difference in the signal magnitude identified at step 6113 of the method 6110 indicates that there is a physical difference between the connection port of the patient interface in use during the first therapy session and the connection port of the patient interface in use during the second therapy session. The existence of a physical difference indicates that the patient interface in use during the second therapy session is not the same patient interface as the patient interface during the first therapy session, which indicates that the patient replaced their patient interface between the first therapy session and the second therapy session.

### 5.9.9.1.2 Multiple Signal Magnitudes

In some particular examples, the first acoustic signature comprises a plurality of first signal magnitudes corresponding to respective first distances from the respiratory pressure therapy device 4000. Likewise the second acoustic signature may comprise a plurality of second signal magnitudes corresponding to respective second distances from the respiratory pressure therapy device 4000.

For example, the acoustic signature may be a set of data points, each data point being a signal magnitude of a reflection from a respective distance from a transducer (e.g. microphone). Each of the three acoustic signatures shown in the FIG. 17 plot comprises a plurality of signature magnitudes (vertical axis) corresponding to respective distances from a microphone of an RPT device (horizontal axis).

In such examples, the step 6111 of acquiring the first acoustic signature (when acquired rather than received) may comprise emitting a first sound from the respiratory pressure therapy device 4000 along an air circuit in use during the first therapy session to the first patient interface. Step 6111 may further comprise detecting a plurality of first reflections of the first sound from a plurality of locations within the first patient interface and then determining a plurality of first signal magnitudes each corresponding to a respective one of the first reflections.

Similarly, the step 6112 of acquiring the second acoustic signature (when acquired rather than received) may comprise emitting a second sound from the respiratory pressure therapy device 4000 along an air circuit in use during the second therapy session to the second patient interface. Step 6112 may further comprise detecting a plurality of second reflections of the sound from a plurality of locations within the second patient interface corresponding to the plurality of locations within the first patient interface, and determining a plurality of second signal magnitudes each corresponding to a respective one of the second reflections.

The step 6113 of identifying the acoustic difference may then comprise identifying one or more differences between the first signal magnitudes and the second signal magnitudes. In some examples, step 6113 may comprise identifying two or more differences, each difference being a difference in signal magnitude between one of the first signal magnitudes and a corresponding one of the second signal magnitudes.

For example, with reference to FIG. 17, during a first therapy session a patient may be using a patient interface identified as Patient Interface A. During step 6111 of the method 6110 an acoustic signature of Patient Interface A may be acquired (for example by an RPT device 4000 providing a flow of gas to Patient Interface A). The acoustic signature for Patient Interface A (a first acoustic signature) is shown in the FIG. 17 plot as a solid line. The acoustic signature may be comprised of many data points corresponding to respective distances from a transducer along an air circuit connected to Patient Interface A and into Patient Interface A itself.

Between the first therapy session and a second therapy session the patient may obtain a new patient interface, identified as Patient Interface B for their treatment and may replace Patient Interface A with Patient Interface B and use Patient Interface B for the first time during the second therapy session. During step 6112 of the method 6110 an acoustic signature of Patient Interface B may be acquired. The acoustic signature for Patient Interface B (a second acoustic signature) is shown as a dotted line in the FIG. 17 plot.

Patient Interface B may have a physical difference to Patient Interface A that produces one or more acoustic differences between the acoustic signatures for Patient Interface A and Patient Interface B. In this particular example, Patient Interface B has a fin in the air flow path located at a connection port, while Patient Interface A has no fin at its connection port. This physical difference (presence of a fin versus absence of a fin), results in differences between the signal magnitudes of reflections of a sound from Patient Interface A compared to the signal magnitudes of reflections of a sound from the connection port in Patient Interface B. As shown in FIG. 17, differences in signal magnitude are identifiable at approximately 1.8 m and 1.95 m distance from the microphone. These are acoustic differences produced as a result of the physical difference of the existence of the fin at the connection port in Patient Interface B but absent in Patient Interface A. The physical difference between Patient Interface A (a first patient interface) and Patient Interface B (a second patient interface) is at a first location within the first patient interface (the connection port of the first patient interface in this example) and at a second location within the second patient interface corresponding to the first location (the connection port of the second patient interface in this example).

It is to be understood that, in practice, two acoustic signatures for the same patient interface, as acquired or received, may not be identical as they may vary due to inaccuracies in generating the emitted sound, receiving and processing a signal for the reflection and noise in each signal. An acoustic difference for the purpose of the present technology is to be understood as a difference resulting from a physical difference in two patient interfaces as opposed to other differences such as may be produced by noise or inaccuracies.

As an acoustic difference between Patient Interface A and Patient Interface B has been identified, it is determined that patient interface replacement has occurred between the therapy session with Patient Interface A and the therapy session with Patient Interface B.

Also shown in FIG. 17 is the acoustic signature of a further patient interface, Patient Interface C which has a longer fin at a connection port 3600 than Patient Interface B. Patient Interface C has a difference in acoustic signature to Patient Interface A at around 2 m from the transducer and a difference in acoustic signature to Patient Interface B at around 1.8 m from the transducer. These differences allow for replacement of Patient Interface A or B with Patient Interface C to be determined. Each patient interface may comprise a fin in its air path having a different shape or size to the other fins. In addition, one patient interface may have no fin. The fins may be located at a straight swivel component of the patient interface. The inventors have found that providing fins proximate another change in cross section (e.g. an elbow component), may reduce accuracy in distinguishing between different fin configurations.

Where it is not possible or desired for a manufacture to produce unique patient interfaces, a number of variants of the same patient interface may be produced (e.g. 3, 4 or more), such that it is more likely than not that if a patient replaces their patient interface with the same model, they will obtain a different variant than the previous variant and patient interface replacement will be detectable by performance of the method 6110.

### 5.9.9.2 Monitoring Method Steps

While method 6110 is a method for determining that patient interface replacement has occurred, in another example of the present technology there is a method 6120 of monitoring for patient interface replacement between therapy sessions of treatment of sleep disordered breathing. Method 6120 may be performed even if patient interface replacement is not detected. FIG. 16B shows a flow chart of method 6120.

In a first step 6121 method 6120 comprises acquiring or receiving a first acoustic signature of a patient interface in use during a first therapy session. In a second step 6122 method 6120 comprises acquiring or receiving second vent flow rate data. Steps 6121 and 6122 may be substantially the same as steps 6111 and 6112 of method 6110. The description in relation to steps 6111 and 6112, including description of the acoustic signatures and how they are acquired or received and including description of particular devices that may perform steps 6111 and 6112, is to be understood to be relevant to steps 6121 and 6122 of method 6120. By way of example only, steps 6121 and 6122 may be performed by an RPT device 4000 providing a pressurised flow of breathable gas to a patient using the patient interface during the therapy sessions. Step 6123 may also be performed by the RPT device 4000 or the method may comprise transmitting the first and second acoustic signatures to a server 9010 or computing device 9040, which may perform step 6123 to check for an acoustic difference between the acoustic signatures.

Method 6120 comprises a third step 6123 of checking for, by comparison of the second acoustic signature to the first acoustic signature, an acoustic difference between the first acoustic signature and the second acoustic signature. The description of step 6113 of method 6110 is also to be understood to be relevant to step 6123 of method 6120, the main difference being that step 6113 comprises identifying an acoustic difference whereas step 6123 comprises checking for an acoustic difference.

While step 6113 of method 6110 may comprise identifying a difference between a first signal magnitude of a first sound reflection and a second signal magnitude of a second sound reflection, in some examples step 6123 of method 6120 comprises checking for a difference between the first signal magnitude and the second signal magnitude.

Similarly, while step 6113 of the method 6110 may comprise identifying one or more differences between a plurality of first signal magnitudes corresponding to a plurality of first sound reflections and a plurality of second signal magnitudes corresponding to a plurality of second sound reflections, in some examples step 6123 of the method 6120 comprises checking for one or more differences between the first signal magnitudes and the second signal magnitudes.

In some examples, step 6123 may comprise checking for two or more differences, each difference being a difference in signal magnitude between one of the first signal magnitudes and a corresponding one of the second signal magnitudes.

Method 6120 may be performed during every therapy session, for example, to monitor for patient interface replacement between therapy sessions. If patient interface replacement is detected (e.g. if the method 6110 is also performed), the RPT device 4000 may record a date at which patient interface replacement occurred, so that the age of the patient interface can subsequently be monitored. If necessary reminders can then be provided if the patient interface reaches an age in which replacement is required.

### 5.9.10 Determining Patient Interface Replacement Based on Patient Input

In a further form of the present technology there is provided a method 6210 for determining that patient interface replacement has occurred. FIG. 18 shows a flow chart of the method 6210, which comprises a first step 6211 of receiving an input from the patient regarding a patient interface in use. Method 6210 also comprises a second step 6212 of determining that the patient interface in use has been entered into use for the first time based on the input. As described elsewhere herein, determining that patient interface replacement has occurred may enable, for example, an age of the patient interface to subsequently be estimated and monitored so that action can be taken when the patient interface reaches a replacement age. With continued use after reaching its service life a patient interface may be more prone to leaks occurring, which may affect the ability of the patient interface to maintain a desired pressure at the patient's airways or may increase noise and/or create an uncomfortable feel on the patient's face.

In some examples, the method 6210 may comprise querying the patient regarding whether the patient interface in use has been entered into use for the first time. The query may be provided to the patient via an RPT device 4000 or a computing device 9040 in use by the patient (e.g. a mobile phone).

The step 6211 of receiving the input may comprise receiving identification information (which may be numeric, alphanumeric or other data, for example). The identification information may indicate that the patient interface in use has been entered into use for the first time. For example, the identification information may be unique to the patient interface in use, meaning the only way the identification information could be provided is if the patient is in possession of the unique patient interface associated with the identification information, indicating that the patient has obtained the patient interface as a replacement for a previous patient interface. In some examples the identification information may be received following the patient scanning a QR code on the patient interface, or a bar code or the like. That is, the patient may, upon obtaining a new patient interface, scan a QR code (or other suitable type of code) provided on or with the patient interface to obtain the identification information represented by the QR code. In some examples the identification information may be automatically transmitted from the device used to scan the QR code to another device (for example the device performing step 6211 of the method). In other examples the identification information may be a number or code that the patient manually enters into the device performing step 6211 (e.g. an RPT device 4000 or computing device 9040 such as a mobile phone or the like).

In some examples, the step 6211 of receiving an input is performed by an RPT device 4000. The step 6212 of determining that the patient interface in use has been entered into use for the first time may also be performed by the RPT device 4000. However, in other examples the step 6212 may be performed by a server 9010 or other computing device 9040. For example, an RPT device 4000 used by a patient may perform step 6211 (e.g. the patient provides an input to the RPT device 4000 regarding a new patient interface) and the input itself, or data representing or generated based on the input may be transmitted to server 9010 or other computing device 9040, which then performs step 6212 to determine that the patient interface has been entered into use for the first time.

In other examples, the step 6211 of receiving an input is performed by a computing device 9040 of the patient (e.g. a smartphone, tablet or the like). This may comprise scanning a QR code or receiving another input, for example an identification number of the patient interface entered manually by a patient. The step 6212 of determining that the patient interface in use has been entered into use for the first time is performed by an RPT device 4000. The patient's computing device 9040 may transmit data representing the input to the RPT device 4000, for example via a Bluetooth connection, wired connection (e.g. USB), or WiFi or other internet connection. In other examples, the step 6122 of determining that the patient interface in use has been entered into use for the first time may be performed by the computing device 9040. In further examples the step 6122 is performed by a server 9010.

### 5.9.11 Estimating Age of Patient Interface Based on Date

Another form of the present technology is a method 6220 of estimating age of a patient interface. FIG. 19 is a flow chart of the method 6220.

In a first step 6221, the method 6220 comprises determining that a patient has entered a patient interface into use for the first time. This step may involve performing another method described herein, such as any method for determining that replacement of a patient interface or a component thereof has occurred, determining that a patient interface or component thereof is new (e.g. based on vent behaviour, other age estimation, patient input or other information).

In particular examples, step 6221 may comprise performing method 6010 for determining that a patient interface component comprising a vent has been replaced between therapy sessions of treatment of sleep disordered breathing, method 6050 for estimating age of a patient interface comprising a vent, method 6060 for determining that a patient interface component comprising an anti-asphyxia valve (AAV) has been replaced between therapy sessions of treatment of sleep disordered breathing, method 6110 for determining that patient interface replacement has occurred based on identification an acoustic difference, method 6210 for determining that patient interface replacement has occurred based on patient input, and/or any other suitable method.

In some examples, the step 6221 of determining that the patient has entered the patient into use for the first time comprises receiving patient interface supply data indicating that the patient has been supplied with a new patient interface. The patient interface supply data may be received from, for example, a clinician or a patient interface supplier.

In some examples, the step 6221 of that the patient has entered the patient into use for the first time comprises identifying a change in duration of therapy session (e.g. usage hours). The change in usage hours may be an increase in usage hours. For example, if the patient obtains a new patient interface they may be more likely to use it for longer than their old one, due to the better performance, comfort and/or appearance of the new mask caused by the lack of wear and tear. Step 6221 may comprise comparing a duration of a first therapy session with a duration of a second therapy session after the first therapy session and identifying that the second therapy session is longer than the first therapy session. Step 6221 may comprise identifying that a difference between the duration of the second therapy session and the duration of the first therapy session is greater than a threshold difference indicating that patient interface replacement occurred between the first therapy session and the second therapy session.

A second step 6222 of the method 6220 comprises recording a date at which the patient interface is entered into use. Step 6222 may be performed, for example, immediately after step 6221 is performed to determining that the patient interface has been entered into use for the first time.

In a third step 6223, the method 6220 comprises estimating an age of the patient interface by comparing a current date with the date at which the patient interface was entered into use. Step 6223 may be performed days, weeks or months, for example, after step 6221 and 6222. In some examples step 6223 is performed during every therapy session to estimate the age of the patient interface in use during the therapy session. Accordingly, while steps 6221 and 6222 may be performed once at the time of identifying a new patient interface, step 6223 may be performed many times afterwards to monitor the age of the patient interface in use.

In some examples, the method 6220 comprises a further step of prompting the patient to replace the patient interface based on the estimated age of the patient interface. For example, the method 6220 may comprise prompting the patient to replace the patient interface if the estimated age of the patient interface is greater than an age at which the patient interface requires replacement. The method 6220 may comprise comparing the estimated age of the patient age to a predetermined replacement age. If the estimated age is greater than or equal to the predetermined replacement age the method 6220 may comprise prompting the patient to replace the patient interface. The predetermined replacement age may be, for example, one month, three months, six months or one year. In some examples, if the estimated age of the patient interface in use is greater than or equal to the predetermined replacement age, the method 6220 may comprise ordering a new patient interface for the patient, shipping a new patient interface to the patient and/or notifying a clinician regarding the estimated age.

Each of the steps of the method 6220 may be performed by an RPT device 4000 in use by the patient or a computing device 9040 operated by the patient. Alternatively each of the steps may be performed by a server 9010 remote from the patient. In some examples one or more of the steps (for example step 6221 and/or 6222) are performed by an RPT device 4000 and subsequent steps (for example step 6223) is performed by a server 9010.

### 5.9.12 Determining Replacement Required Based on Counter

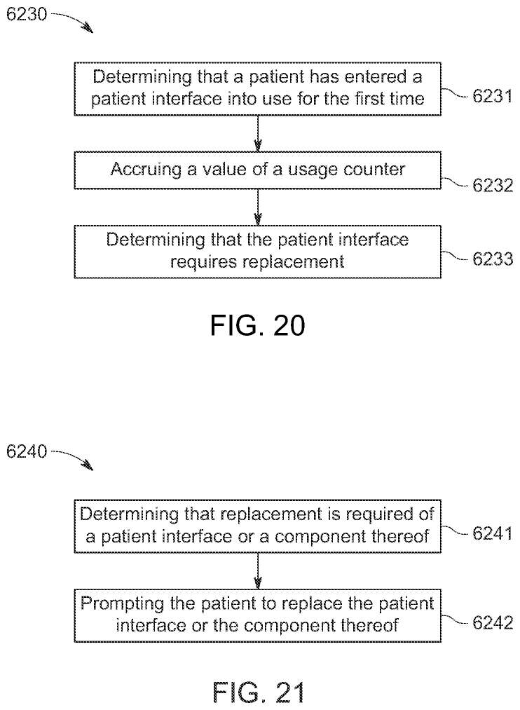

Another form of the present technology is a method 6230 of determining that a patient interface in use requires replacement. FIG. 20 shows a flow chart of the method.

In a first step 6231 the method 6230 may comprise determining that a patient has entered a patient interface into use for the first time. This step may involve performing another method described herein, such as any method for determining that replacement of a patient interface or a component thereof has occurred, determining that a patient interface or component thereof is new (e.g. based on vent behaviour, other age estimation, patient input or other information).

In particular examples, step 6231 may comprise performing method 6010 for determining that a patient interface component comprising a vent has been replaced between therapy sessions of treatment of sleep disordered breathing, method 6050 for estimating age of a patient interface comprising a vent, method 6060 for determining that a patient interface component comprising an anti-asphyxia valve (AAV) has been replaced between therapy sessions of treatment of sleep disordered breathing, method 6110 for determining that patient interface replacement has occurred based on identification an acoustic difference, method 6210 for determining that patient interface replacement has occurred based on patient input, and/or any other suitable method.

A second step 6232 of the method 6230 may comprise accruing a value of a usage counter, the usage counter representing an amount of use of the patient interface. In some examples the method 6230 may first comprise zeroing the value of the usage counter after determining that the patient has entered the patient interface into use.

The value of the usage counter may represent a property of the patient interface or a patient interface component which changes over time with usage of the patient interface. The usage counter, in various examples of the present technology, may represent a number of days of use of the patient interface, a number of usage hours of the patient interface, and/or a number of therapy sessions since patient interface replacement occurred.

In a third step 6233 the method 6230 may comprise determining that the patient interface requires replacement based at least partially on the value of the usage counter. For example, if the usage counter reflects an amount of use greater than the service life of the patient interface, it may be determined at step 6233 that the patient interface requires replacement.

In comparison to estimating an age of a patient interface based on dates, a usage counter may also reflect the amount of use of a patient interface between two particular dates. For example, for two patient interfaces replaced one month ago, one may have been used nightly while the other may have been used only every second night or only on weeknights. Through the use of a usage counter the method 6230 may determine that the patient interface used nightly requires replacement sooner than the patient interface that is used sporadically.

The step 6233 of determining that the patient interface requires replacement comprises comparing the value of the usage counter to a threshold value. The threshold value may represent an amount of usage at which it is determined that patient interface replacement is required. For example, if a patient interface has a replacement interval of three months the threshold value may be three months, or may two and a half months in order to allow the patient time to obtain a new patient interface. In some examples, the method 6230 comprises providing reminders to the patient to obtain a new patient interface as the value of the usage counter approaches the threshold value. Other methods of determining that patient interface replacement is required, such as method 6220 as one example, may also comprise providing reminders to the patient as the requirement for patient interface replacement approaches.

In some examples the method 6230 comprises accruing a value of a supplementary usage counter, the supplementary usage counter being representative of an amount of use of the patient interface. The step 6233 of determining that the patient interface requires replacement may comprise comparing the value of the supplementary usage counter to a supplementary threshold value. The supplementary usage counter may be a second usage counter which may supplement the aforementioned usage counter (which may be a first usage counter) as it may provide for a second aspect of usage to be accounted for. For example, a value of the first usage counter may represent a number of days of use of the patient interface and a value of the second usage counter may represent a number of usage hours of the patient interface. Step 6233 may comprise determining that replacement is required either when the value of the first usage counter reaches a threshold value or when the value of the supplementary usage counter reaches a supplementary threshold value.

In some examples, if the value of the usage counter is greater than or equal to a threshold value, the method 6230 may comprise ordering a new patient interface for the patient, shipping a new patient interface to the patient and/or notifying a clinician regarding the estimated age. Such steps may be performed by an RPT device 4000 in use by the patient or a server 9010.

Each of the steps of the method 6230 may be performed by an RPT device 4000 in use by the patient or a computing device 9040 operated by the patient. Alternatively each of the steps may be performed by a server 9010 remote from the patient. In some examples one or more of the steps (for example step 6231 and/or 6232) are performed by an RPT device 4000 and subsequent steps (for example step 6233) is performed by a server 9010 or a computing device 9040.

### 5.9.13 Prompting Patient Interface Replacement

Another form of the present technology is a method 6240 of prompting a patient to replace a patient interface or component thereof. FIG. 21A shows a flow chart of the method.

In a first step 6241, the method 6240 may comprise determining that replacement is required of patient interface or a component thereof in use by a patient during a therapy session for treatment of sleep disordered breathing. Any method disclosed herein of determining that patient interface or component replacement is required may be performed at step 6241 to determine that patient interface replacement is required.

In a second step 6242, the method 6240 may comprise prompting the patient to replace the patient interface or the component thereof.

The step 6241 of determining that replacement is required may be performed by a RPT device 4000 providing a pressurised flow of breathable gas to the patient interface during the therapy session. The step 6242 of prompting the patient may also be performed by the RPT device 4000, for example by a message on a display of the RPT device 4000 or with an audible message. Alternatively, the step 6252 may be performed by a computing device 9040 operated by the patient, for example a mobile phone notification or via an email readable on a personal computer or mobile communication device.

In other examples the step 6241 of determining that replacement is required may be performed by a server 9010 or computing device 9040 with which an RPT device 4000 providing a pressurised flow of breathable gas to the patient interface during the therapy session is configured to communicate. The step 6242 of prompting the patient may then be performed by the RPT device 4000 or a computing device 9040 operated by the patient.

### 5.9.14 Facilitating Patient Interface Replacement

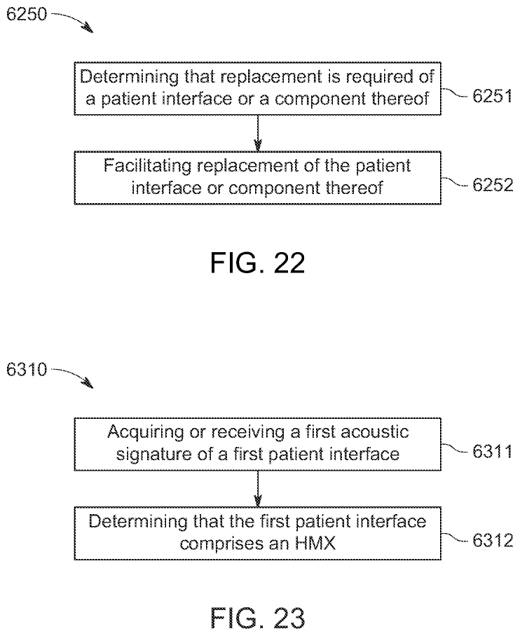

Another form of the present technology is a method 6250 for facilitating patient interface replacement. FIG. 22 shows a flow chart of the method 6250. A first step 6251 may comprise determining that replacement is required of a patient interface or a component thereof. Step 6251 may comprise any method described herein of determining that patient interface or component replacement is required and may be performed by an RPT device 4000 or may be performed by a server 9010 or computing device 9040 (e.g. a mobile communication device operated by the patient).

A second step 6252 of the method 6250 may comprise facilitating replacement of the patient interface or the component thereof. Facilitating replacement may comprise ordering a replacement patient interface or component thereof, notifying a third party that replacement of the patient interface is required (e.g. a supplier of patient interfaces or a clinician) or any other way of beginning, completing or otherwise facilitating the patient interface replacement process. In some examples step 6252 comprises shipping a patient interface or component to the patient and/or delivering a patient interface or component thereof to the patient. In some examples, the method 6252 comprises receiving an input from a patient regarding an action to be taken to facilitate patient interface replacement, such as confirmation to order to a new patient interface or a setting in a user profile to enable automatic mask replacement.

Step 6252 may be performed by an RPT device 4000 or by a server 9010 or communication device 9040 (for example a mobile phone). In some examples the step 6251 of determining that replacement is required may be performed by an RPT device and step 6252 may be performed by a server 9010 or mobile communication device 9040 operated by the patient.

5.9.15 Determining that an HMX is in Use

Another form of the present technology is a method 6310 for determining that a patient interface in use by a patient for treatment of sleep disordered breathing comprises a heat and moisture exchanger (HMX). An HMX may comprise a material held in the air flow path configured to adsorb moisture from a patient's breath during exhalation and desorb it to the air to be inhaled, thereby humidifying the air prior to inhalation using moisture from exhaled air. In some examples the HMX may be formed from corrugated paper. In other examples the HMX may be formed from CaCl₂ treated polyurethane foam.

Method 6310 may comprise a first step 6311 of acquiring or receiving a first acoustic signature of a first patient interface in use during a therapy session. The first acoustic signature may be acquired or received as described above in relation to methods 6110 and/or 6120. In other examples the first acoustic signature is acquired by a different method.

Method 6310 may then comprise a second step 6312 of determining, based on the first acoustic signature, that the first patient interface comprises an HMX.

In some examples of the method 6310, step 6311 comprises acquiring the first acoustic signature (as opposed to receiving it). The step 6311 may be performed by a respiratory pressure therapy device 4000 operatively connected to the first patient interface during a therapy session. Step 6312 may also be performed by the RPT device 4000 in some examples.

The first acoustic signature may comprise one or more first signal magnitudes of one or more respective detected reflections of a first sound from one or more first distances from the RPT device 4000 along an air circuit 4170 in use during the therapy session and into the first patient interface. More details of properties of signal magnitudes, acoustic signatures and how an acoustic signature may be acquired are included herein in the description of other methods involving acoustic signatures, such as the methods 6110 and 6120. In particular, the first acoustic signature may comprise a plurality of first signal magnitudes corresponding to respective first distances from the respiratory pressure therapy device 4000. Any acquired acoustic signatures and reference signal magnitudes may be normalised such that one acoustic signature can be directly compared to another and/or to a reference acoustic signature.

FIG. 25 shows three acoustic signatures of a patient interface: an acoustic signature (solid line) of a patient interface having no HMX fitted, an acoustic signature (broken line) of a patient interface having a first model of HMX named HMX 1, and an acoustic signature (dotted line), of a patient interface fitted with a second model of an HMX named HMX 2. Each acoustic signature comprises a plurality of signal magnitudes corresponding to respective distances from a transducer of an RPT device 4000.

In some examples the sound emitted along the air conduit 4170 is (or approximates) an impulse. The acoustic signature detected may comprise (e.g. may approximate) an impulse response function. In some examples, the method comprises identifying one or more features of the acoustic signature indicating the presence of an HMX. If the sound emitted along an air circuit 4170 towards a patient interface is short/sharp (e.g. an impulse), and the size of the HMX is small in comparison to its spacing from the plenum chamber of the patient interface, it may be distinguishable in the acoustic signature by one or more characteristics (e.g. a particular signal magnitude corresponding to a particular distance from the transducer or a particular shape in the acoustic signature). If the sound emitted is not short enough in duration or the HMX is too close to other detectable portions of the patient interface the HMX may not be distinguishable as reflections of the emitted sound from the HMX may overlap in time with reflections from other detectable portions of the patient interface. Where the emitted sound is or approximates an impulse, the acoustic signature may be identified as an impulse response. The acoustic signature may comprise data representing detected signal magnitude as a function of time or distance from a transducer. The acoustic signature may comprise one or more features (e.g. characteristics such as a shape or a particular signal magnitude) indicating the presence of an HMX in the air circuit.

In some examples, the step 6132 of determining that the first patient interface comprises an HMX comprises comparing one of the first signal magnitudes corresponding to an expected distance from the respiratory pressure therapy device of the HMX with a reference signal magnitude. The reference signal magnitude may have a value indicating the presence of an HMX and the step 6132 of determining that the first patient interface comprises an HMX may comprise identifying that the first signal magnitude corresponding to the expected distance of the HMX is substantially the same as the reference signal magnitude. Alternatively, the reference signal magnitude has a value indicating the absence of an HMX and the step of determining that the first patient interface comprises an HMX comprises identifying that the first signal magnitude corresponding to the expected distance of the HMX is not substantially equal to the reference signal magnitude.

With reference to FIG. 25, a reference signal magnitude for a patient interface with no HMX may be about 0.005, which occurs in this case at about 2.2 m from the transducer at an RPT device 4000. A reference signal magnitude for the patient interface with HMX 1 at about 2.2 m from the transducer is 0.15 and a reference signal magnitude for the patient interface with HMX 2 at about 2.2 m from the transducer is 0.1. Accordingly, in some examples of the method 6310 the reference signal magnitude is 0.1 (or alternately, or additionally, 0.15), indicating the presence of an HMX, and the step 6312 comprises identifying that the signal magnitude of the acquired acoustic signature at 2.2 m is 0.1 (substantially the same as the reference signal), indicating the presence of an HMX. Alternatively, in some examples of the method 6310 the reference signal magnitude is 0.005, indicating the absence of an HMX, and the step 6312 comprises identifying that the signal magnitude of the acquired signal is not 0.005, thereby indicating the presence of an HMX.

Once it has been determined by the method 6310 that an HMX is present in the air circuit 4170, the method 6310 may comprise reminding that patient to replace the HMX. For example the method 6310 may comprise providing monthly reminders to the patient to replace their HMX. The method 6310 may comprise providing reminders at a frequency corresponding to the service life of an HMX.

If an HMX is detected, the method 6310 may also comprise disabling humidification, if active humidification is being used by the patient (e.g. using a humidifier 5000 with a supply water to be vapourised into the flow of gas). In particular, the method 6310 may comprise disabling active humidification of the pressurised flow of breathable gas to the patient interface from the RPT device 4000. In another example, the method 6310 may comprise prompting the patient to disable active humidification, if an HMX is detected in the system.

In some examples, the method 6310 may comprises determining that the patient interface in use comprises an HMX and determining a distance along an air circuit 4170 of the HMX from the transducer. The method 6310 may comprise identifying one or more characteristics of a signal of a reflection of a sound emitted into an air circuit 4170 towards a patient interface indicating the presence and location of an HMX. For example, with reference to FIG. 25, the method 6310 may comprise analysing a signal of a reflected sound to identify signal behaviour indicating the presence of an HMX. The signal behaviour may be represented visually as a particular shape on a plot of signal magnitude against distance from the transducer indicating that the sound reflected from around a location was reflected from an HMX (e.g. the shape visible in FIG. 25 within the 2-2.5 m distance from the transducer). In some examples the method 6310 may comprise identifying frequency(ies) and/or amplitude(s) of a signal of a sound reflected from a patient interface indicating presence of an HMX. In some examples the method 6310 comprises performing statistical analysis on a signal of a sound reflected from a patient interface to determine a probability of the presence of an HMX and determining that an HMX is present based on the probability. In some examples the method 6310 comprises processing a signal of a sound reflected from a patient interface along an air circuit 4170 with a machine learning model to identify the presence of an HMX in the air circuit.

In some examples, the method 6310 may comprise identifying the distance of a patient interface 3000 from an RPT device 4000 by analysis of the acquired signal of a sound reflected from the patient interface 3000 along the air circuit 4170. The patient interface 3000 may be identified by detection of characteristics of the signal indicative of the presence of an HMX, including for example frequencies and/or amplitudes. In some examples the type of patient interface (e.g. full face, ultracompact full face, nasal, pillows, conduit headgear etc.) and/or specific model of patient interface 3000 may be determined. Detection of the patient interface and determining its distance from the transducer at the RPT device 4000 may improve accuracy in detecting presence of an HMX as an HMX is typically close to the patient interface 3000 to keep volume of potentially rebreathed air low.

### 5.9.15.1 Detection of HMX Saturation

In some examples, in addition to detecting that the patient interface in use includes an HMX, the method 6310 may comprise determining that the HMX is saturated. An HMX saturated with moisture may reflect sound back to a transducer at the RPT device 4000 differently than a dry HMX.

FIG. 24 shows three acoustic signatures of a patient interface: an acoustic signature (solid line) of a patient interface fitted with a dry HMX, an acoustic signature (dotted line) of a patient interface fitted with a saturated HMX and an acoustic signature (broken line) of a patient interface fitted with a saturated HMX+ (a model of an HMX optimised for particularly dry ambient conditions). Each acoustic signature comprises a plurality of signal magnitudes corresponding to respective distances from a transducer of an RPT device 4000.

As shown in FIG. 24, the signal magnitudes at the location of the HMX (about 2.25 m from the transducer) differ for each of the dry HMX, saturated HMX and the saturated HMX+. The method 6310 may comprise comparing the acquired (or received, as the case may be) signal magnitude at the location of the HMX to a plurality of reference signal magnitudes (for example reference signal magnitudes for a dry HMX, saturated HMX an saturated HMX+). The method 6310 may then comprise determining whether the acquired signal magnitude indicates a dry HMX, saturated HMX or saturated HMX+, for example by determining whether the acquired signal magnitude is closest in magnitude to a reference signal magnitude for a dry HMX, saturated HMX or saturated HMX+. Alternative approaches (e.g. statistical analysis and/or application of a machine learning model) may also form part of a method 6310 able to distinguish between a dry HMX and a saturated HMX or HMX+.

In some examples, if it is determined by the method 6310 that an HMX is present and is saturated, the method 6310 may further comprise prompting the patient to remove and/or replace the HMX. Alternatively, or additionally, the method 6310 may comprise heating at least a portion of the air circuit 4170 (for example if the patient is using a conduit that can be heated) or increasing a temperature of or heating power provided to heat the air circuit 4170. This may reduce condensation and the amount of moisture retained by the HMX. The method 6310 may alternatively or additionally comprise disabling or reducing active humidification if the patient is using a humidifier 5000, which may reduce the absolute humidity of the air provided to the patient. In some examples, if it is determined that an HMX+ is present in the air circuit 4170 and that it is saturated, the method 6310 may comprise prompting the patient to replace the HMX+ with an HMX configured to retain less moisture than the HMX+ (e.g. a regular model HMX, not an HMX+ which is configured for very dry conditions).

### 5.9.15.2 Devices Performing Method 6310

While in some examples an RPT device 4000 providing therapy to a patient may perform both steps 6311 and 6312 to detect presence of an HMX, in some examples of the method 6310 the RPT device 4000 may perform step 6311 and then the method 6310 may comprise transmitting the first acoustic signature to a server 9010 or other remote computing device 9040. The RPT device 4000 may transmit the first acoustic signature to a server 9010 or other remove computing device 9040. The step 6312 of determining that the first patient interface comprises the HMX may then be performed by the server 9010 or other remote computing device 9040. For example, the server 9010 or computing device 9040 remote from the patient may have reference signal magnitudes and/or reference acoustic signatures stored in memory for accessing when performing step 6312.

In some examples, the method 6310 comprises transmitting the first acoustic signature to a computing device 9040 operated by the patient (for example a mobile phone) and the step 6312 of determining that the first patient interface comprises the HMX is performed by the computing device 9040. In some examples the computing device 9040 operated by the patient may exchange data over a communication network 9030 with a server 9010, for example to obtain reference signal magnitudes. Where a server 9010 or computing device 9040 is performing the method 6310, the step 6311 may comprise receiving the first acoustic signature (as opposed to acquiring it).

### Glossary

For the purposes of the present technology disclosure, in certain forms of the present technology, one or more of the following definitions may apply. In other forms of the present technology, alternative definitions may apply.

### 5.9.16 General

Air: In certain forms of the present technology, air may be taken to mean atmospheric air, and in other forms of the present technology air may be taken to mean some other combination of breathable gases, e.g. atmospheric air enriched with oxygen.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy (RPT): The application of a supply of air to an entrance to the airways at a treatment pressure that is typically positive with respect to atmosphere.

Ventilator: A mechanical device that provides pressure support to a patient to perform some or all of the work of breathing.

### 5.9.17 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:

(i) Flattened: Having a rise followed by a relatively flat portion, followed by a fall.

(ii) M-shaped: Having two local peaks, one at the leading edge, and one at the trailing edge, and a relatively flat portion between the two peaks.

(iii) Chair-shaped: Having a single local peak, the peak being at the leading edge, followed by a relatively flat portion.

(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

(inhalation) Time (Ti): The duration of the inspiratory portion of the respiratory flow rate waveform.

(exhalation) Time (Te): The duration of the expiratory portion of the respiratory flow rate waveform.

(total) Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.10 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A method for determining that a patient interface component comprising a vent has been replaced between therapy sessions of treatment of sleep disordered breathing, the method comprising:
acquiring or receiving first vent flow rate data, the first vent flow rate data representing one or more estimated first vent flow rates of gas through a first vent of a patient interface in use during a first therapy session;
acquiring or receiving second vent flow rate data, the second vent flow rate data representing one or more estimated second vent flow rates of gas through a second vent of a patient interface in use during a second therapy session after the first therapy session; and
identifying, by comparison of the second vent flow rate data to the first vent flow rate data, a difference in resistance to flow through the first vent than through the second vent indicating that the second vent is not the same vent as the first vent.

2. The method of claim 1, wherein the difference in resistance to flow is a greater resistance to flow through the second vent than through the first vent.

3. The method of claim 1, wherein the first vent flow rate data represents a plurality of estimated first vent flow rates each corresponding to a respective one of a plurality of therapy pressures, and the second vent flow rate data represents a plurality of estimated second vent flow rates each corresponding to a respective one of the plurality of therapy pressures.

4. The method of claim 3, wherein the method comprises identifying the difference in resistance to flow by determining that for each one of the plurality of therapy pressures, the corresponding second vent flow rate is different to the corresponding first vent flow rate.

5. The method of claim 1, wherein the first vent flow rate data represents an estimated first vent flow rate corresponding to a predetermined therapy pressure, and the second vent flow rate data represents an estimated second vent flow rate corresponding to the predetermined therapy pressure.

6. The method of claim 5, wherein the method comprises determining that the second vent flow rate is different to the first vent flow rate.

7. The method of claim 1, wherein the step of acquiring or receiving the first vent flow rate data comprises acquiring the first vent flow rate data and the step of acquiring or receiving the second vent flow rate data comprises acquiring the second vent flow rate data, wherein the steps of acquiring the first vent flow rate data and acquiring the second vent flow rate data are performed by a respiratory pressure therapy device providing a pressurised flow of breathable gas to the patient interface in use during the first therapy session and to the patient interface in use during the second therapy session.

8. The method of claim 7, wherein the step of identifying the difference in resistance is performed by the respiratory pressure therapy device.

9. The method of claim 7, wherein the method comprises transmitting the first vent flow rate data and the second vent flow rate data to a server, and the step of identifying the difference in resistance is performed by a server.

10. The method of claim 1, wherein the step of acquiring or receiving the first vent flow rate data comprises receiving the first vent flow rate data and the step of acquiring or receiving the second vent flow rate data comprises receiving the second vent flow rate data, wherein the steps of receiving the first vent flow rate data and receiving the second vent flow rate data are performed by a server, and the step of identifying the difference in resistance is performed by the server.

11. A method for monitoring for replacement of a patient interface component comprising a vent between therapy sessions of treatment of sleep disordered breathing, the method comprising:
acquiring or receiving first vent flow rate data during a first therapy session, the first vent flow rate data representing one or more estimated first vent flow rates of gas through a vent of a patient interface in use during the first therapy session;
acquiring or receiving second vent flow rate data during a second therapy session after the first therapy session, the second vent flow rate data representing one or more estimated second vent flow rates of gas through a vent of a patient interface in use during the second therapy session; and
checking for, by comparison of the second vent flow rate data to the first vent flow rate data, a difference in resistance to flow through the vent of the patient interface in use during the second therapy session than through the vent of the patient interface in use during the first therapy session.

12. The method of claim 11, wherein the difference in resistance to flow is a greater resistance to flow through the vent of the patient interface in use during the second therapy session than through the vent of the patient interface in use during the first therapy session.

13. The method of claim 11, wherein the first vent flow rate data represents a plurality of estimated first vent flow rates each corresponding to a respective one of a plurality of therapy pressures, and the second vent flow rate data represents a plurality of estimated second vent flow rates each corresponding to a respective one of the plurality of therapy pressures.

14. The method of claim 13, wherein the step of checking for the difference in resistance to flow comprises checking for a difference, for each one of the plurality of therapy pressures, between the corresponding second vent flow rate and the corresponding first vent flow rate.

15. The method of claim 11, wherein the first vent flow rate data represents an estimated first vent flow rate corresponding to a predetermined therapy pressure, and the second vent flow rate data represents an estimated second vent flow rate corresponding to the predetermined therapy pressure.

16. The method of claim 15, wherein the step of checking for the difference in resistance to flow comprises checking for a difference between the second flow rate and the first flow rate.

17. The method of claim 11, wherein the step of acquiring or receiving the first vent flow rate data comprises acquiring the first vent flow rate data and the step of acquiring or receiving the second vent flow rate data comprises acquiring the second vent flow rate data, wherein the steps of acquiring the first vent flow rate data and acquiring the second vent flow rate data are performed by a respiratory pressure therapy device providing a pressurised flow of breathable gas to the patient interface in use during the first therapy session and to the patient interface in use during the second therapy session.

18. The method of claim 17, wherein the step of checking for the difference in resistance to flow is performed by the respiratory pressure therapy device.

19. The method of claim 17, wherein the method comprises transmitting the first vent flow rate data and the second vent flow rate data to a server, and the step of checking for the difference in resistance to flow is performed by the server.

20. The method of claim 11, wherein the step of acquiring or receiving the first vent flow rate data comprises receiving the first vent flow rate data and the step of acquiring or receiving the second vent flow rate data comprises receiving the second vent flow rate data, wherein the steps of receiving the first vent flow rate data and receiving the second vent flow rate data are performed by a server, and the step of checking for the difference in resistance to flow is performed by the server.
